(19) United States
(12) Patent Application Publication
(10) Pub. No.: US 2025/0034548 A1
(43) Pub. Date: Jan. 30, 2025

(54) IMPROVED MODULAR PRIME EDITING WITH MODIFIED EFFECTORS AND TEMPLATES

(71) Applicant: UNIVERSITY OF MASSACHUSETTS, Boston, MA (US)

(72) Inventors: Erik Joseph Sontheimer, Auburndale, MA (US); Jonathan Kenneth Watts, Worcester, MA (US); Wen Xue, Natick, MA (US); Zexiang Chen, Worcester, MA (US); Bin Liu, Boston, MA (US)

(21) Appl. No.: 18/678,835

(22) Filed: May 30, 2024

Related U.S. Application Data
(60) Provisional application No. 63/469,897, filed on May 31, 2023.

(51) Int. Cl. C12N 15/10; C12N 9/12; C12N 9/22; C12N 15/11

(52) U.S. Cl. CPC C12N 15/102; C12N 9/1241; C12N 9/22; C12N 15/111; C07K 2319/70; C12N 2310/531

(57) ABSTRACT

Provided are modular prime editing systems, comprising: i) a fusion protein comprising a Cas9 nickase protein linked to a nucleotide polymerase (NT) protein, ii) a prime editor template RNA (petRNA) comprising a primer binding site (PBS), a nucleotide polymerase template (NPT), and at least one MS2 hairpin, and iii) a single guide RNA (sgRNA).

### CROSS REFERENCE TO RELATED APPLICATION

This application claims the benefit of U.S. Provisional Patent Application Ser. No. 63/469,897, filed May 31, 2023. The entire content of the above-referenced patent application is incorporated by reference in its entirety herein.

### SEQUENCE LISTING

The instant application contains a Sequence Listing which has been submitted electronically in XML format and is hereby incorporated by reference in its entirety. Said XML file, created on Sep. 26, 2024, is named 753315_UM9-297_ST26.xml and is 183,097 bytes in size.

### FIELD

The disclosure relates to modular prime editing platforms comprising of a fusion protein comprising a Cas9 nickase (nCas9) linked to a nucleotide polymerase (NP) protein and a separate prime editor template RNA (pegRNA) and methods of use of the same.

### BACKGROUND

Correction of genetic mutations in vivo has broad potential therapeutic application for a range of human genetic diseases. Prime editors (PE) composed of a nCas9 fused to an engineered NP have enabled precise nucleotide changes, sequence insertions and deletions. Anzalone et al., “Search-and-replace genome editing without double-strand breaks or donor DNA” Nature 576:149-157 (2019).

This innovative technology does not induce double-stranded DNA breaks and does not require a donor DNA template in conjunction with homology directed repair to introduce precise sequence changes into the genome. The ability to precisely install or correct pathogenic mutations makes prime editors an excellent tool to perform somatic genome editing.

Unlike base editing systems, prime editors can introduce any nucleotide substitution as well as insertions and deletions, and do not suffer from the challenges of bystander base conversion. These abilities may provide important advantages in some sequence contexts. Prime editor consists of a nCas9 (H840A)-NP fusion protein paired with a pegRNA with desired edits. However, base editing efficiencies can be low.

Accordingly, there exists a need in the art for improved prime editors.

### SUMMARY

The subject specification provides a modular prime editing system.

In certain aspects, provided herein is a modular prime editing system, comprising: i) a fusion protein comprising a Cas9 nickase protein linked to a nucleotide polymerase (NT) protein, ii) a prime editor template RNA (petRNA) comprising a primer binding site (PBS), a nucleotide polymerase template (NPT), and at least one MS2 hairpin, and iii) a single guide RNA (sgRNA), wherein the fusion protein comprises at least one MS2 binding protein inlaid within the Cas9 nickase.

In some embodiments, the fusion protein comprises two or more MS2 binding proteins inlaid within the Cas9 nickase.

In some embodiments, the fusion protein comprises two or more adjacent MS2 binding proteins inlaid within the Cas9 nickase.

In some embodiments, the fusion protein comprises two or more nonadjacent MS2 binding proteins inlaid within the Cas9 nickase.

In some embodiments, the fusion protein consists of two MS2 binding proteins inlaid within the Cas9 nickase.

In some embodiments, the fusion protein consists of two adjacent MS2 binding proteins inlaid within the Cas9 nickase.

In some embodiments, the fusion protein consists of two nonadjacent MS2 binding proteins inlaid within the Cas9 nickase.

In some embodiments, the fusion protein comprises two MS2 binding proteins inlaid within one of the Rec-1, RuvC-III, PID, or HNH domains of the Cas9 nickase.

In some embodiments, the fusion protein comprises two adjacent MS2 binding proteins inlaid within one of the Rec-1, RuvC-III, PID, or HNH domains of the Cas9 nickase.

In some embodiments, the fusion protein comprises two nonadjacent MS2 binding proteins inlaid within one of the Rec-1, RuvC-III, PID, or HNH domains of the Cas9 nickase.

In some embodiments, wherein the fusion protein comprises two MS2 binding proteins inlaid within the PID domain of the Cas9 nickase.

In some embodiments, the fusion protein comprises two adjacent MS2 binding proteins inlaid within the PID domain of the Cas9 nickase.

In some embodiments, the fusion protein comprises two nonadjacent MS2 binding proteins inlaid within the PID domain of the Cas9 nickase.

In some embodiments, the fusion protein comprises two MS2 binding proteins inlaid within position G1247 of the PID domain of the Cas9 nickase of SEQ ID NO: 1.

In some embodiments, the fusion protein comprises two adjacent MS2 binding proteins inlaid within position G1247 of the PID domain of the Cas9 nickase of SEQ ID NO: 1.

In some embodiments, the fusion protein comprises two nonadjacent MS2 binding proteins inlaid within position G1247 of the PID domain of the Cas9 nickase of SEQ ID NO: 1.

In some embodiments, the one or more MS2 binding proteins are attached to the Cas9 nickase via one or more linkers.

In some embodiments, wherein the one or more MS2 binding proteins are attached to the Cas9 nickase via two linkers.

In some embodiments, the one or more MS2 binding proteins are attached to the Cas9 nickase via two linkers, wherein a first linker is on the N-terminus of the Cas9 nickase, and a second linker is on the C-terminus of the Cas9 nickase.

In some embodiments, the one or more MS2 binding proteins are attached to each other via one or more linker.

In some embodiments, the one or more MS2 binding proteins are attached to each other via one linker and to the Cas9 nickase via two linkers.

In some embodiments, the one or more MS2 binding proteins are attached to each other via one linker and to the Cas9 nickase via two linkers, wherein the first linker is on the N-terminus of the Cas9 nickase, and the second linker is on the C-terminus of the Cas9 nickase.

In some embodiments, the two MS2 binding proteins inlaid within the Cas9 nickase are attached to each other via one linker and to the Cas9 nickase via two linkers, wherein the first linker is on the N-terminus of the Cas9 nickase, and the second linker is on the C-terminus of the Cas9 nickase.

In some embodiments, the fusion protein comprises from the N-terminus to the C-terminus: the N-terminus portion of the Cas9 nickase protein, one MS2 binding protein, the C-terminus portion of the Cas9 nickase protein, and an NT protein; or the N-terminus portion of the Cas9 nickase protein, two MS2 binding proteins, the C-terminus portion of the Cas9 nickase protein, and an NT protein.

In some embodiments, the fusion protein comprises from the N-terminus to the C-terminus: the N-terminus portion of the Cas9 nickase protein, a first linker, one MS2 binding protein, a second linker, the C-terminus portion of the Cas9 nickase protein, a third linker, and an NT protein; or the N-terminus portion of the Cas9 nickase protein, a first linker, a first MS2 binding protein, a second linker, a second MS2 binding protein, a third linker, the C-terminus portion of the Cas9 nickase protein, a fourth linker, and an NT protein.

In some embodiments, the Cas9 nickase comprises one or more amino acid substitution.

In some embodiments, the one or more amino acid substitution in the Cas9 nickase is an H840A substitution.

In some embodiments, the modular prime editing system comprises: the N-terminus portion of the Cas9 nickase protein comprising the sequence of SEQ ID NO: 2; the MS2 binding protein comprising the sequence of SEQ ID NO: 21; the C-terminus portion of the Cas9 nickase protein comprising the sequence of SEQ ID NO:11; and the NT protein comprising the sequence of SEQ ID NO: 19.

In some embodiments, the modular prime editing system comprises: the N-terminus portion of the Cas9 nickase protein comprising the sequence of SEQ ID NO: 3; the first MS2 binding protein comprising the sequence of SEQ ID NO: 21; the second MS2 binding protein comprising the sequence of SEQ ID NO: 21; the C-terminus portion of the Cas9 nickase comprising the sequence of SEQ ID NO: 12; and the NT protein comprising the sequence of SEQ ID NO: 19.

In some embodiments, the modular prime editing system comprises: the N-terminus portion of the Cas9 nickase protein comprising the sequence of SEQ ID NO: 4; the first MS2 binding protein comprising the sequence of SEQ ID NO: 21; the second MS2 binding protein comprising the sequence of SEQ ID NO: 21; the C-terminus portion of the Cas9 nickase comprising the sequence of SEQ ID NO: 13; and the NT protein comprising the sequence of SEQ ID NO: 19.

In some embodiments, the modular prime editing system comprises: the N-terminus portion of the Cas9 nickase protein comprising the sequence of SEQ ID NO: 5; the first MS2 binding protein comprising the sequence of SEQ ID NO: 21; the second MS2 binding protein comprising the sequence of SEQ ID NO: 21; the C-terminus portion of the Cas9 nickase comprising the sequence of SEQ ID NO: 14; and the NT protein comprising the sequence of SEQ ID NO: 19.

In some embodiments, the modular prime editing system comprises: the N-terminus portion of the Cas9 nickase protein comprising the sequence of SEQ ID NO: 6; the first MS2 binding protein comprising the sequence of SEQ ID NO: 21; the second MS2 binding protein comprising the sequence of SEQ ID NO: 21; the C-terminus portion of the Cas9 nickase comprising the sequence of SEQ ID NO: 15; and the NT protein comprising the sequence of SEQ ID NO: 19.

In some embodiments, the modular prime editing system comprises: the N-terminus portion of the Cas9 nickase protein comprising the sequence of SEQ ID NO: 7; the first MS2 binding protein comprising the sequence of SEQ ID NO: 21; the second MS2 binding protein comprising the sequence of SEQ ID NO: 21; the C-terminus portion of the Cas9 nickase comprising the sequence of SEQ ID NO: 16; and the NT protein comprising the sequence of SEQ ID NO: 19.

In some embodiments, the modular prime editing system comprises: the N-terminus portion of the Cas9 nickase protein comprising the sequence of SEQ ID NO: 8; the first MS2 binding protein comprising the sequence of SEQ ID NO: 21; the second MS2 binding protein comprising the sequence of SEQ ID NO: 21; the C-terminus portion of the Cas9 nickase comprising the sequence of SEQ ID NO: 17; and the NT protein comprising the sequence of SEQ ID NO: 19.

In some embodiments, the modular prime editing system comprises: the N-terminus portion of the Cas9 nickase protein comprising the sequence of SEQ ID NO: 2; the first linker comprising the sequence of SEQ ID NO: 31; the MS2 binding protein comprising the sequence of SEQ ID NO: 21; the second linker comprising the sequence of SEQ ID NO: 32; the C-terminus portion of the Cas9 nickase protein comprising the sequence of SEQ ID NO: 11; the third linker comprising the sequence of SEQ ID NO: 26; and the NT protein comprising the sequence of SEQ ID NO: 19.

In some embodiments, the modular prime editing system comprises: the N-terminus portion of the Cas9 nickase protein comprising the sequence of SEQ ID NO: 3; the first linker comprising the sequence of SEQ ID NO: 34; the first MS2 binding protein comprising the sequence of SEQ ID NO: 21; the second linker comprising the sequence of SEQ ID NO: 31; the second MS2 binding protein comprising the sequence of SEQ ID NO: 21; the third linker comprising the sequence of SEQ ID NO: 33; the C-terminus portion of the Cas9 nickase comprising the sequence of SEQ ID NO: 12; the fourth linker comprising the sequence of SEQ ID NO: 26; and the NT protein comprising the sequence of SEQ ID NO: 19.

In some embodiments, the modular prime editing system comprises: the N-terminus portion of the Cas9 nickase protein comprising the sequence of SEQ ID NO: 4; the first linker comprising the sequence of SEQ ID NO: 34; the first MS2 binding protein comprising the sequence of SEQ ID NO: 21; the second linker comprising the sequence of SEQ ID NO: 31; the second MS2 binding protein comprising the sequence of SEQ ID NO: 21; the third linker comprising the sequence of SEQ ID NO: 33; the C-terminus portion of the Cas9 nickase comprising the sequence of SEQ ID NO: 13; the fourth linker comprising the sequence of SEQ ID NO: 26; and the NT protein comprising the sequence of SEQ ID NO: 19.

In some embodiments, the modular prime editing system comprises: the N-terminus portion of the Cas9 nickase protein comprising the sequence of SEQ ID NO: 5; the first linker comprising the sequence of SEQ ID NO: 34; the first MS2 binding protein comprising the sequence of SEQ ID NO: 21; the second linker comprising the sequence of SEQ ID NO: 31; the second MS2 binding protein comprising the sequence of SEQ ID NO: 21; the third linker comprising the sequence of SEQ ID NO: 33; the C-terminus portion of the Cas9 nickase comprising the sequence of SEQ ID NO: 14; the fourth linker comprising the sequence of SEQ ID NO: 26; and the NT protein comprising the sequence of SEQ ID NO: 19.

In some embodiments, the modular prime editing system comprises: the N-terminus portion of the Cas9 nickase protein comprising the sequence of SEQ ID NO: 6; the first linker comprising the sequence of SEQ ID NO: 34; the first MS2 binding protein comprising the sequence of SEQ ID NO: 21; the second linker comprising the sequence of SEQ ID NO: 31; the second MS2 binding protein comprising the sequence of SEQ ID NO: 21; the third linker comprising the sequence of SEQ ID NO: 33; the C-terminus portion of the Cas9 nickase comprising the sequence of SEQ ID NO: 15; the fourth linker comprising the sequence of SEQ ID NO: 26; and the NT protein comprising the sequence of SEQ ID NO: 19.

In some embodiments, the modular prime editing system comprises: the N-terminus portion of the Cas9 nickase protein comprising the sequence of SEQ ID NO: 7; the first linker comprising the sequence of SEQ ID NO: 34; the first MS2 binding protein comprising the sequence of SEQ ID NO: 21; the second linker comprising the sequence of SEQ ID NO: 31; the second MS2 binding protein comprising the sequence of SEQ ID NO: 21; the third linker comprising the sequence of SEQ ID NO: 33; the C-terminus portion of the Cas9 nickase comprising the sequence of SEQ ID NO: 16; the fourth linker comprising the sequence of SEQ ID NO: 26; and the NT protein comprising the sequence of SEQ ID NO: 19.

In some embodiments, the modular prime editing system comprises: the N-terminus portion of the Cas9 nickase protein comprising the sequence of SEQ ID NO: 8; the first linker comprising the sequence of SEQ ID NO: 34; the first MS2 binding protein comprising the sequence of SEQ ID NO: 21; the second linker comprising the sequence of SEQ ID NO: 31 the second MS2 binding protein comprising the sequence of SEQ ID NO: 21; the third linker comprising the sequence of SEQ ID NO: 33; the C-terminus portion of the Cas9 nickase comprising the sequence of SEQ ID NO: 17; the fourth linker comprising the sequence of SEQ ID NO: 26; and the NT protein comprising the sequence of SEQ ID NO: 19.

In some embodiments, the fusion protein comprises the sequences of SEQ ID NOS: 43, 44, 45, 46, 47, 48, and 49.

In certain aspects, provided herein is a modular prime editing system comprising: i) a fusion protein comprising a Cas9 nickase protein linked to a nucleotide polymerase (NT) protein; ii) a prime editor template RNA (petRNA) comprising a primer binding site, a nucleotide polymerase template (NPT), and at least one MS2 hairpin; and iii) a single guide RNA (sgRNA), wherein the fusion protein comprises at least four MS2 binding proteins.

In some embodiments, the fusion protein consists of four MS2 binding proteins.

In some embodiments, the fusion protein consists of four adjacent MS2 binding proteins.

In some embodiments, the fusion protein consists of four nonadjacent MS2 binding proteins.

In some embodiments, the fusion protein consists of four adjacent MS2 binding proteins on the N-terminus.

In some embodiments, the fusion protein consists of four nonadjacent MS2 binding proteins on the N-terminus.

In some embodiments, the fusion protein consists of four adjacent MS2 binding proteins on the C-terminus.

In some embodiments, the fusion protein consists of four nonadjacent MS2 binding proteins on the C-terminus.

In some embodiments, the fusion protein consists of two adjacent MS2 binding proteins on the N-terminus, and two MS2 binding proteins on the C-terminus.

In some embodiments, the fusion protein consists of two adjacent MS2 binding proteins on the N-terminus, and two adjacent MS2 binding proteins on the C-terminus.

In some embodiments, the fusion protein consists of two adjacent MS2 binding proteins on the N-terminus, and two nonadjacent MS2 binding proteins on the C-terminus.

In some embodiments, the fusion protein consists of two nonadjacent MS2 binding proteins on the N-terminus, and two MS2 binding proteins on the C-terminus.

In some embodiments, the fusion protein consists of two nonadjacent MS2 binding proteins on the N-terminus, and two adjacent MS2 binding proteins on the C-terminus.

In some embodiments, the fusion protein consists of two nonadjacent MS2 binding proteins on the N-terminus, and two nonadjacent MS2 binding proteins on the C-terminus.

In some embodiments, the fusion protein consists of two MS2 binding proteins on the N-terminus, and two MS2 binding proteins inlaid in the Cas9 nickase.

In some embodiments, the fusion protein consists of two adjacent MS2 binding proteins on the N-terminus, and two MS2 binding proteins inlaid in the Cas9 nickase.

In some embodiments, the fusion protein consists of two nonadjacent MS2 binding proteins on the N-terminus, and two MS2 binding proteins inlaid in the Cas9 nickase.

In some embodiments, the fusion protein consists of two MS2 binding proteins on the N-terminus, and two adjacent MS2 binding proteins inlaid in the Cas9 nickase.

In some embodiments, the fusion protein consists of two MS2 binding proteins on the N-terminus, and two nonadjacent MS2 binding proteins inlaid in the Cas9 nickase.

In some embodiments, the fusion protein consists of two MS2 binding proteins on the C-terminus, and two MS2 binding proteins inlaid in the Cas9 nickase.

In some embodiments, the fusion protein consists of two adjacent MS2 binding proteins on the C-terminus, and two MS2 binding proteins inlaid in the Cas9 nickase.

In some embodiments, the fusion protein consists of two nonadjacent MS2 binding proteins on the C-terminus, and two MS2 binding proteins inlaid in the Cas9 nickase.

In some embodiments, the fusion protein consists of two MS2 binding proteins on the C-terminus, and two adjacent MS2 binding proteins inlaid in the Cas9 nickase.

In some embodiments, the fusion protein consists of two MS2 binding proteins on the C-terminus, and two nonadjacent MS2 binding proteins inlaid in the Cas9 nickase.

In some embodiments, the fusion protein consists of two MS2 binding proteins on the N-terminus, and two MS2 binding proteins inlaid within one of the Rec-1, RuvC-III, PID, or HNH domains of the Cas9 nickase.

In some embodiments, the fusion protein consists of two adjacent MS2 binding proteins on the N-terminus, and two MS2 binding proteins inlaid within one of the Rec-1, RuvC-III, PID, or HNH domains of the Cas9 nickase.

In some embodiments, the fusion protein consists of two nonadjacent MS2 binding proteins on the N-terminus, and two MS2 binding proteins inlaid within one of the Rec-1, RuvC-III, PID, or HNH domains of the Cas9 nickase.

In some embodiments, the fusion protein consists of two MS2 binding proteins on the N-terminus, and two adjacent MS2 binding proteins inlaid within one of the Rec-1, RuvC-III, PID, or HNH domains of the Cas9 nickase.

In some embodiments, the fusion protein consists of two adjacent MS2 binding proteins on the N-terminus, and two nonadjacent MS2 binding proteins inlaid within one of the Rec-1, RuvC-III, PID, or HNH domains of the Cas9 nickase.

In some embodiments, the fusion protein consists of two MS2 binding proteins on the N-terminus, and two MS2 binding proteins inlaid within the PID domain of the Cas9 nickase.

In some embodiments, the fusion protein consists of two adjacent MS2 binding proteins on the N-terminus, and two MS2 binding proteins inlaid within the PID domain of the Cas9 nickase.

In some embodiments, the fusion protein consists of two nonadjacent MS2 binding proteins on the N-terminus, and two MS2 binding proteins inlaid within the PID domain of the Cas9 nickase.

In some embodiments, the fusion protein consists of two MS2 binding proteins on the N-terminus, and two adjacent MS2 binding proteins inlaid within the PID domain of the Cas9 nickase.

In some embodiments, the fusion protein consists of two MS2 binding proteins on the N-terminus, and two nonadjacent MS2 binding proteins inlaid within the PID domain of the Cas9 nickase.

In some embodiments, the fusion protein consists of two MS2 binding proteins on the N-terminus, and two MS2 binding proteins inlaid within position G1247 of the PID domain of the Cas9 nickase of SEQ ID NO: 1.

In some embodiments, the fusion protein consists of two adjacent MS2 binding proteins on the N-terminus, and two MS2 binding proteins inlaid within position G1247 of the PID domain of the Cas9 nickase of SEQ ID NO: 1.

In some embodiments, the fusion protein consists of two nonadjacent MS2 binding proteins on the N-terminus, and two MS2 binding proteins inlaid within position G1247 of the PID domain of the Cas9 nickase of SEQ ID NO: 1.

In some embodiments, the fusion protein consists of two MS2 binding proteins on the N-terminus, and two adjacent MS2 binding proteins inlaid within position G1247 of the PID domain of the Cas9 nickase of SEQ ID NO: 1.

In some embodiments, the fusion protein consists of two MS2 binding proteins on the N-terminus, and two nonadjacent MS2 binding proteins inlaid within position G1247 of the PID domain of the Cas9 nickase of SEQ ID NO: 1.

In some embodiments, the at least four MS2 binding proteins are attached to the Cas9 nickase via one or more linker.

In some embodiments, the at least four MS2 binding proteins are attached to the Cas9 nickase via two linkers.

In some embodiments, the at least four MS2 binding proteins are attached to the Cas9 nickase via two linkers, wherein a first linker is on the N-terminus of the Cas9 nickase, and a second linker is on the C-terminus of the Cas9 nickase.

In some embodiments, the at least four MS2 binding proteins are attached to each other via one or more linker.

In some embodiments, the at least four MS2 binding proteins are attached to each other via one or more linker and to the Cas9 nickase via one or more linker.

In some embodiments, the at least four MS2 binding proteins are attached to each other via one linker and to the Cas9 nickase via two linkers.

In some embodiments, the at least four MS2 binding proteins are attached to each other via one linker and to the Cas9 nickase via two linkers, wherein the first linker is on the N-terminus of the Cas9 nickase, and the second linker is on the C-terminus of the Cas9 nickase.

In some embodiments, the fusion protein comprises from the N-terminus to the C-terminus: Four adjacent MS2 binding proteins, the Cas9 nickase protein, and an NT protein; or A first MS2 binding protein, a second MS2 binding protein, the Cas9 nickase protein, an NT protein, a third MS2 binding protein and a fourth MS2 binding protein; or A first MS2 binding protein, a second MS2 binding protein, the N-terminus portion of the Cas9 nickase protein, a third MS2 binding protein and a fourth MS2 binding protein, the C-terminus portion of the Cas9 nickase protein, and an NT protein; or The Cas9 nickase protein, an NT protein, and four adjacent MS2 binding proteins.

In some embodiments, the fusion protein comprises from the N-terminus to the C-terminus: A first MS2 binding protein, a first linker, a second MS2 binding protein, a second linker, a third MS2 protein, a third linker, a fourth MS2 protein, a fourth linker, the Cas9 nickase protein, a fifth linker, and an NT protein; or A first MS2 binding protein, a first linker, a second MS2 binding protein, a second linker, the Cas9 nickase protein, a third linker, an NT protein, a fourth linker, a third MS2 binding protein, a fifth linker, and a fourth MS2 protein; or A first MS2 binding protein, a first linker, a second MS2 binding protein, a second linker, the N-terminus portion of the Cas9 nickase protein, a third linker, a third MS2 binding protein, a fourth linker, a fourth MS2 protein, a fifth linker, the C-terminus portion of the Cas9 nickase protein, and an NT protein, or The Cas9 nickase protein, a first linker, and an NT protein, a second linker, a first MS2 binding protein, a third linker, a second MS2 binding protein, a fourth linker, a third MS2 protein, a fifth linker, and a fourth MS2 protein.

In some embodiments, the Cas9 nickase comprises one or more amino acid substitution.

In some embodiments, the one or more amino acid substitution in the Cas9 nickase is an H840A substitution.

In some embodiments, the modular prime editing system comprises: the first MS2 binding protein comprises the sequence of SEQ ID NO: 21; the second MS2 binding protein comprises the sequence of SEQ ID NO: 21; the third MS2 binding protein comprises the sequence of SEQ ID NO: 21; the fourth MS2 binding protein comprises the sequence of SEQ ID NO: 21; the Cas9 nickase protein comprise the sequence of SEQ ID NO: 1; and the NT comprises the sequence of SEQ ID NO: 19.

In some embodiments, the modular prime editing system comprises: the first MS2 binding protein comprises the sequence of SEQ ID NO: 21; the second MS2 binding protein comprises the sequence of SEQ ID NO: 21 the Cas9 nickase protein comprise the sequence of SEQ ID NO: 1; the NT comprises the sequence of SEQ ID NO: 19; the third MS2 binding protein comprises the sequence of SEQ ID NO: 21; and the fourth MS2 binding protein comprises the sequence of SEQ ID NO: 21.

In some embodiments, the modular prime editing system comprises: the first MS2 binding protein comprises the sequence of SEQ ID NO: 21; the second MS2 binding protein comprises the sequence of SEQ ID NO: 21; the N-terminus portion of the Cas9 nickase protein comprises the sequence of SEQ ID NO: 9; the third MS2 binding protein comprises the sequence of SEQ ID NO: 21; the fourth MS2 binding protein comprises the sequence of SEQ ID NO: 21; the C-terminus portion of the Cas9 nickase protein comprises the sequence of SEQ ID NO: 18; and the NT comprises the sequence of SEQ ID NO: 19.

In some embodiments, the modular prime editing system comprises: the first MS2 binding protein comprises the sequence of SEQ ID NO: 21; the first linker comprises the sequence of SEQ ID NO: 31; the second MS2 binding protein comprises the sequence of SEQ ID NO: 21; the second linker comprises the sequence of SEQ ID NO: 33; the third MS2 binding protein comprises the sequence of SEQ ID NO: 21; the third linker comprises the sequence of SEQ ID NO: 31; the fourth MS2 binding protein comprises the sequence of SEQ ID NO: 21; the fourth linker comprises the sequence of SEQ ID NO: 30; the Cas9 nickase protein comprise the sequence of SEQ ID NO: 1; the fifth linker comprises the sequence of SEQ ID NO: 26; and the NT comprises the sequence of SEQ ID NO: 19.

In some embodiments, the modular prime editing system comprises: the first MS2 binding protein comprises the sequence of SEQ ID NO: 21; the first linker comprises the sequence of SEQ ID NO: 31; the second MS2 binding protein comprises the sequence of SEQ ID NO: 21; the second linker comprises the sequence of SEQ ID NO: 30; the Cas9 nickase protein comprise the sequence of SEQ ID NO: 1; the third linker comprises the sequence of SEQ ID NO: 26; the NT comprises the sequence of SEQ ID NO: 19; the fourth linker comprises the sequence of SEQ ID NO: 34; the third MS2 binding protein comprises the sequence of SEQ ID NO: 21; the fifth linker comprises the sequence of SEQ ID NO: 31; and the fourth MS2 binding protein comprises the sequence of SEQ ID NO: 21.

In some embodiments, the modular prime editing system comprises: the first MS2 binding protein comprises the sequence of SEQ ID NO: 21; the first linker comprises the sequence of SEQ ID NO: 31; the second MS2 binding protein comprises the sequence of SEQ ID NO: 21; the second linker comprises the sequence of SEQ ID NO: 30; the N-terminus portion of the Cas9 nickase protein comprises the sequence of SEQ ID NO: 9; the third linker comprises the sequence of SEQ ID NO: 34; the third MS2 binding protein comprises the sequence of SEQ ID NO: 21; the fourth linker comprises the sequence of SEQ ID NO: 31; the fourth MS2 binding protein comprises the sequence of SEQ ID NO: 21; the fifth linker comprises the sequence of SEQ ID NO: 30; the C-terminus portion of the Cas9 nickase protein comprises the sequence of SEQ ID NO: 18; the sixth linker comprises the sequence of SEQ ID NO: 26; and the NT comprises the sequence of SEQ ID NO: 19.

In some embodiments, the fusion protein comprises the sequences of SEQ ID NOS: 50, 51, and 52.

In certain aspects, provided herein is a modular prime editing system comprising: i) a fusion protein comprising a Cas9 nickase protein linked to a nucleotide polymerase (NT) protein; ii) a prime editor template RNA (petRNA) comprising a primer binding site, a nucleotide polymerase template (NPT), and at least one MS2 hairpin; and iii) a single guide RNA (sgRNA), wherein the fusion protein comprises at least one MS2 binding protein at the N terminus and at least one MS2 binding protein at the C terminus.

In some embodiments, the fusion protein consists of one MS2 binding protein on the N-terminus, and one MS2 binding protein on the C-terminus.

In some embodiments, the at least one MS2 binding protein at the N terminus and at least one MS2 binding protein at the C terminus are attached to the Cas9 nickase via one or more linker.

In some embodiments, the at least one MS2 binding protein at the N terminus and at least one MS2 binding protein at the C terminus are attached to the Cas9 nickase via two linkers.

In some embodiments, the at least one MS2 binding protein at the N terminus and at least one MS2 binding protein at the C terminus are attached to the Cas9 nickase via two linkers, wherein a first linker is on the N-terminus of the Cas9 nickase, and a second linker is on the C-terminus of the Cas9 nickase.

In some embodiments, the fusion protein comprises from the N-terminus to the C-terminus: A first MS2 binding protein, the Cas9 nickase protein, an NT protein, and a second MS2 binding protein.

In some embodiments, the fusion protein comprises from the N-terminus to the C-terminus: A first MS2 binding protein, a first linker, the Cas9 nickase protein, a second linker, an NT protein, a third linker, and a second MS2 binding protein.

In some embodiments, the Cas9 nickase comprises one or more amino acid substitution.

In some embodiments, the one or more amino acid substitution in the Cas9 nickase is an H840A substitution.

In some embodiments, the modular prime editing system comprises: the first MS2 binding protein comprises the sequence of SEQ ID NO: 21; the Cas9 nickase protein comprises the sequence of SEQ ID NO: 1; the NT comprises the sequence of SEQ ID NO: 19; and the second MS2 binding protein comprises the sequence of SEQ ID NO: 21.

In some embodiments, the modular prime editing system comprises: the first MS2 binding protein comprises the sequence of SEQ ID NO: 21 the first linker comprises the sequence of SEQ ID NO: 30; the Cas9 nickase protein comprises the sequence of SEQ ID NO: 1; the second linker comprises the sequence of SEQ ID NO: 26; the NT comprises the sequence of SEQ ID NO: 19; the third linker comprises the sequence of SEQ ID NO: 26; and the second MS2 binding protein comprises the sequence of SEQ ID NO: 21.

In some embodiments, the fusion protein comprises the sequence of SEQ ID NO: 42.

In certain aspects, provided herein is a modular prime editing system comprising i) a fusion protein comprising a Cas9 nickase protein linked to a nucleotide polymerase (NT) protein; ii) a prime editor template RNA (petRNA) comprising a primer binding site, a nucleotide polymerase template (NPT), and at least one MS2 hairpin; and iii) a single guide RNA (sgRNA), wherein the fusion protein comprises at least one MS2 binding protein at the N terminus or at least one MS2 binding protein at the C terminus or at least one MS2 binding protein between the Cas9 nickase and the RT.

In some embodiments, the fusion protein consists of one MS2 binding protein on the N-terminus.

In some embodiments, the fusion protein consists of two MS2 binding proteins on the N-terminus.

In some embodiments, the fusion protein consists of one MS2 binding protein on the N-terminus and one MS2 binding protein between the Cas9 nickase and the RT.

In some embodiments, the fusion protein consists of one MS2 binding protein on the C-terminus.

In some embodiments, the fusion protein consists of one MS2 binding protein on the C-terminus and one MS2 binding protein between the Cas9 nickase and the RT.

In some embodiments, the fusion protein consists of one MS2 binding protein between the Cas9 nickase and the RT.

In some embodiments, the at least one MS2 binding protein at the N terminus or at least one MS2 binding protein at the C terminus or at least one MS2 binding protein between the Cas9 nickase and the NT are attached to the Cas9 nickase via one or more linker.

In some embodiments, the at least one MS2 binding protein at the N terminus or at least one MS2 binding protein at the C terminus or at least one MS2 binding protein between the Cas9 nickase and the NT are attached to NT via one or more linker.

In some embodiments, at least one MS2 binding protein at the N terminus or at least one MS2 binding protein at the C terminus or at least one MS2 binding protein between the Cas9 nickase and the NT are attached to the Cas9 nickase via a first linker and to the NT via a second linker.

In some embodiments, at least one MS2 binding protein at the N terminus or at least one MS2 binding protein at the C terminus or at least one MS2 binding protein between the Cas9 nickase and the NT are attached to the Cas9 nickase via a first linker and to the NT via a second linker, wherein the first linker is on the N-terminus of the Cas9 nickase, and a second linker is on the C-terminus of the RT.

In some embodiments, the fusion protein comprises from the N-terminus to the C-terminus: An MS2 binding protein, the Cas9 nickase protein, and an NT protein; or The Cas9 nickase protein, the NT protein, and an MS2 binding protein; or The Cas9 nickase protein, an MS2 binding protein, and the NT protein.

In some embodiments, the fusion protein comprises from the N-terminus to the C-terminus: The MS2 binding protein, a first linker, the Cas9 nickase protein, a second linker and an NT protein; or The Cas9 nickase protein, a first linker, the NT protein, a second linker, and an MS2 binding protein; or The Cas9 nickase protein, a first linker, an MS2 binding protein, a second linker, and the NT protein.

In some embodiments, the Cas9 nickase comprises one or more amino acid substitution.

In some embodiments, the one or more amino acid substitution in the Cas9 nickase is an H840A substitution.

In some embodiments, the modular prime editing system comprises: the MS2 binding protein comprises the sequence of SEQ ID NO: 21; the Cas9 nickase protein comprises the sequence of SEQ ID NO: 1; and the NT comprises the sequence of SEQ ID NO: 19.

In some embodiments, the modular prime editing system comprises: the first MS2 binding protein comprises the sequence of SEQ ID NO: 21; the second MS2 binding protein comprises the sequence of SEQ ID NO: 21; the Cas9 nickase protein comprises the sequence of SEQ ID NO: 1; and the NT comprises the sequence of SEQ ID NO: 19.

In some embodiments, the modular prime editing system comprises: the Cas9 nickase protein comprises the sequence of SEQ ID NO: 1; the MS2 binding protein comprises the sequence of SEQ ID NO: 21; and the NT comprises the sequence of SEQ ID NO: 19.

In some embodiments, the modular prime editing system comprises: the Cas9 nickase protein comprises the sequence of SEQ ID NO: 1; the NT comprises the sequence of SEQ ID NO: 19; and the MS2 binding proteins comprises the sequence of SEQ ID NO: 21.

In some embodiments, the modular prime editing system comprises: the MS2 binding proteins comprises the sequence of SEQ ID NO: 21; the first linker comprises the sequence of SEQ ID NO: 30; the Cas9 nickase protein comprises the sequence of SEQ ID NO: 1; the second linker comprises the sequence of SEQ ID NO: 26; and the NT comprises the sequence of SEQ ID NO: 19.

In some embodiments, the modular prime editing system comprises: the Cas9 nickase protein comprises the sequence of SEQ ID NO: 1; the first linker comprises the sequence of SEQ ID NO: 31; the MS2 binding proteins comprises the sequence of SEQ ID NO: 21; the second linker comprises the sequence of SEQ ID NO: 26; and the NT comprises the sequence of SEQ ID NO: 19.

In some embodiments, the modular prime editing system comprises: the first MS2 binding proteins comprises the sequence of SEQ ID NO: 21; the first linker comprises the sequence of SEQ ID NO: 31; the second MS2 binding proteins comprises the sequence of SEQ ID NO: 21; the second linker comprises the sequence of SEQ ID NO: 30; the Cas9 nickase protein comprises the sequence of SEQ ID NO: 1; the third linker comprises the sequence of SEQ ID NO: 26; and the NT comprises the sequence of SEQ ID NO: 19.

In some embodiments, the modular prime editing system comprises: the Cas9 nickase protein comprises the sequence of SEQ ID NO: 1; the first linker comprises the sequence of SEQ ID NO: 26; the NT comprises the sequence of SEQ ID NO: 19; the second linker comprises the sequence of SEQ ID NO: 26, and the MS2 binding proteins comprises the sequence of SEQ ID NO: 21.

In some embodiments, the fusion protein comprises the sequence of SEQ ID NO: 38, 39, 40, or 41.

In some embodiments, the nucleotide polymerase is selected from the group consisting of deoxyribonucleic acid polymerase protein (DNAPol), ribonucleic acid polymerase protein (RNAPol), a deoxyribonucleic acid nucleotide polymerase template (dNPT), a ribonucleic acid nucleotide polymerase template (rNPT), and a reverse transcriptase RT.

In some embodiments, the nucleotide polymerase is an RT.

In some embodiments, the nucleotide polymerase is a Moloney murine leukemia virus RT (M-MLV RT).

In some embodiments, the petRNA is chemically modified.

In some embodiments, the one or more modified nucleotides comprise a modification of a ribose group, a phosphate group, a nucleobase, or a combination thereof.

In some embodiments, the modification of the ribose group is selected from 2′-O-methyl, 2′-fluoro, 2′-deoxy, 2′-O-(2-methoxyethyl) (MOE), or 2′-NH2.

In some embodiments, the modification of the phosphate group comprises a phosphorothioate, phosphonoacetate (PACE), thiophosphonoacetate (thioPACE), amide, triazole, phosphonate, or phosphotriester modification.

In some embodiments, the modified phosphate group comprises at least one phosphorothioate internucleotide linkage.

In some embodiments, the modified phosphate group comprises two phosphorothioate internucleotide linkages.

In some embodiments, the modified phosphate group comprises three phosphorothioate internucleotide linkages.

In some embodiments, the modified phosphate group comprises at least one phosphorothioate internucleotide linkage in the PBS.

In some embodiments, the modified phosphate group consists of two phosphorothioate internucleotide linkages in the PBS.

In some embodiments, the modified phosphate group consists of three phosphorothioate internucleotide linkages in the PBS.

In some embodiments, the modification of the nucleobase group is selected from 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups.

In some embodiments, said petRNA comprises one MS2 hairpin.

In some embodiments, said petRNA comprises two MS2 hairpins.

In some embodiments, said petRNA comprises two adjacent MS2 hairpins.

In some embodiments, said petRNA comprises three MS2 hairpins.

In some embodiments, said petRNA comprises four MS2 hairpins.

In some embodiments, the at least one MS2 hairpin is chemically modified.

In some embodiments, the one or more modified nucleotides of the MS2 hairpin comprises a modification of a ribose group, a phosphate group, a nucleobase, or a combination thereof.

In some embodiments, the modified MS2 hairpin comprises a phosphate group comprising a phosphorothioate, phosphonoacetate (PACE), thiophosphonoacetate (thioPACE), amide, triazole, phosphonate, or phosphotriester modification.

In some embodiments, the modified MS2 hairpin comprises a phosphate group comprising at least one phosphorothioate internucleotide linkage.

In some embodiments, the modified MS2 hairpin comprises a phosphate group comprising three phosphorothioate internucleotide linkages.

In some embodiments, the modified MS2 hairpin comprises a phosphate group comprising ten phosphorothioate internucleotide linkages.

In some embodiments, the modified MS2 hairpin comprises a phosphate group comprising twenty-three phosphorothioate internucleotide linkages.

In some embodiments, the phosphorothioate internucleotide linkages are located on the N terminus.

In some embodiments, the phosphorothioate internucleotide linkages are located on the C terminus.

In some embodiments, the at least one MS2 hairpin is fully chemically modified.

In certain aspects, provided herein is a fusion protein comprising a Cas9 nickase protein linked to a nucleotide polymerase (NT) protein, wherein the fusion protein comprises at least one MS2 binding protein inlaid within said Cas9 nickase.

In some embodiments, said fusion protein consists of four MS2 binding proteins.

In some embodiments, said fusion protein consists of four adjacent MS2 binding proteins.

In some embodiments, said fusion protein consists of four nonadjacent MS2 binding proteins.

In some embodiments, said fusion protein consists of four adjacent MS2 binding proteins on the N-terminus.

In some embodiments, said fusion protein consists of four nonadjacent MS2 binding proteins on the N-terminus.

In some embodiments, said fusion protein consists of four adjacent MS2 binding proteins on the C-terminus.

In some embodiments, said fusion protein consists of four nonadjacent MS2 binding proteins on the C-terminus.

In some embodiments, said fusion protein consists of two adjacent MS2 binding proteins on the N-terminus, and two adjacent MS2 binding proteins on the C-terminus.

In some embodiments, said fusion protein consists of two MS2 nonadjacent binding proteins on the N-terminus, and two nonadjacent MS2 binding proteins on the C-terminus.

In some embodiments, said fusion protein consists of two adjacent MS2 binding proteins on the N-terminus, and two nonadjacent MS2 binding proteins on the C-terminus.

In some embodiments, said fusion protein consists of two nonadjacent MS2 binding proteins on the N-terminus, and two adjacent MS2 binding proteins on the C-terminus.

In some embodiments, said fusion protein consists of two MS2 binding proteins in sequence on the N-terminus, and two MS2 binding proteins inlaid in the Cas9 nickase.

In some embodiments, said fusion protein consists of two adjacent MS2 binding proteins on the N-terminus, and two adjacent MS2 binding proteins inlaid in the Cas9 nickase.

In some embodiments, said fusion protein consists of two MS2 binding proteins on the C-terminus, and two MS2 binding proteins inlaid in the Cas9 nickase.

In some embodiments, said fusion protein consists of two adjacent MS2 binding proteins on the C-terminus, and two MS2 binding proteins inlaid in the Cas9 nickase.

In some embodiments, said fusion protein consists of two adjacent MS2 binding proteins on the C-terminus, and two adjacent MS2 binding proteins inlaid in the Cas9 nickase.

In some embodiments, said fusion protein consists of two MS2 binding proteins on the N-terminus, and two MS2 binding proteins inlaid within one of the Rec-1, RuvC-III, PID, or HNH domains of the Cas9 nickase.

In some embodiments, said fusion protein consists of two adjacent MS2 binding proteins on the N-terminus, and two adjacent MS2 binding proteins inlaid within one of the Rec-1, RuvC-III, PID, or HNH domains of the Cas9 nickase.

In some embodiments, said fusion protein consists of two adjacent MS2 binding proteins on the N-terminus, and two MS2 binding proteins inlaid within one of the Rec-1, RuvC-III, PID, or HNH domains of the Cas9 nickase.

In some embodiments, said fusion protein consists of two MS2 binding proteins on the N-terminus, and two adjacent MS2 binding proteins inlaid within one of the Rec-1, RuvC-III, PID, or HNH domains of the Cas9 nickase.

In some embodiments, said fusion protein consists of two adjacent MS2 binding proteins on the N-terminus, and two adjacent MS2 binding proteins inlaid within the PID domain of the Cas9 nickase.

In some embodiments, said fusion protein consists of two adjacent MS2 binding proteins on the N-terminus, and two adjacent MS2 binding proteins inlaid within position G1247 of the PID domain of the Cas9 nickase of SEQ ID NO: 1.

In some embodiments, the MS2 binding proteins are inlaid at the Rec-1, RuvC, PID, HNH, and G1247 positions of the Cas9 nickase of SEQ ID NO 1.

In some embodiments, the fusion protein comprises two MS2 binding proteins, one at the N terminus and one at the C terminus.

In some embodiments, the fusion protein comprises at least one nuclear localization signal (NLS).

In some embodiments, the NLS is on the N-terminus of the Cas9 nickase.

In some embodiments, the NLS is on the C-terminus of the RT.

In some embodiments, the NLS is on the C-terminus of the MCP binding protein.

In some embodiments, the fusion protein comprises two NLS.

In some embodiments, the NLS is on the N-terminus of the Cas9 nickase, and the second NLS is on the C-terminus of the RT.

In some embodiments, the NLS is on the N-terminus of the Cas9 nickase, and the second NLS is on the C-terminus of the MCP binding protein.

In some embodiments, the NLS comprises PKKKRKV (SEQ ID NO:24).

In some embodiments, the NLS comprises the sequences of SEQ ID NOs: 22-25.

In some embodiments, the NLS further comprises a 3×FLAG sequence.

In some embodiments, the disclosure provides a polynucleotide sequence encoding any of the fusion proteins described herein.

In some embodiments, the polynucleotide sequence is an mRNA.

In some embodiments, the mRNA comprises a vector.

In some embodiments, the vector is a viral vector.

In some embodiments, the viral vector is an adeno-associated virus (AAV) vector or a lentivirus (LV) vector.

In some embodiments, the disclosure provides a host cell comprising the vector described herein.

In some embodiments, provided herein is a method of delivering the modular prime editing described herein to a cell, the method comprising incubating the modular prime editing with the cell.

In some embodiments, the fusion protein is delivered as an mRNA.

In some embodiments, the target gene is selected from the list comprising of: EXM1, HEXA, IDUA, HBB, VEGFA, RUNX1, PSEN1, IDS, FANCF, PRNP, and DNMT1.

In some embodiments, provided herein is a method of editing a target gene in a cell, comprising administering to said cell the modular prime editing system described herein.

In some embodiments, the fusion protein of the modular prime editing system is delivered as an mRNA.

In some embodiments, the target gene is selected from the list comprising of: EXM1, HEXA, IDUA, HBB, VEGFA, RUNX1, PSEN1, IDS, FANCF, PRNP, and DNMT1.

In some embodiments, the sgRNA comprises from N-terminus to C-terminus a variable spacer sequence and a common scaffold sequence.

In some embodiments, the common scaffold sequence is

(SEQ ID NO: 106)
GUUUUAGAGCUAGAAAUAGCAAGUUAAAAUAAGGCUAGUCCGUUAUCAAC
UUGAAAAAGUGGCACCGAGUCGGUGC.

In some embodiments, the variable spacer sequence is selected from the sequences of SEQ ID(s) NO(s): 54-86.

In certain aspects, provided herein is a petRNA a comprising a primer binding site, a nucleotide polymerase template (NPT), at least one MS2 hairpin, and at least one chemically modified nucleotide.

In some embodiments, the one or more modified nucleotides comprise a modification of a ribose group, a phosphate group, a nucleobase, or a combination thereof.

In some embodiments, the modification of the ribose group is selected from 2′-O-methyl, 2′-fluoro, 2′-deoxy, 2′-O-(2-methoxyethyl) (MOE), or 2′-NH2.

In some embodiments, the modification of the phosphate group comprises a phosphorothioate, phosphonoacetate (PACE), thiophosphonoacetate (thioPACE), amide, triazole, phosphonate, or phosphotriester modification.

In some embodiments, the modified phosphate group comprises at least one phosphorothioate internucleotide linkage.

In some embodiments, the modified phosphate group comprises two phosphorothioate internucleotide linkages.

In some embodiments, the modified phosphate group comprises three phosphorothioate internucleotide linkages.

In some embodiments, the modified phosphate group comprises at least one phosphorothioate internucleotide linkage on the PBS.

In some embodiments, the modified phosphate group comprises exactly two phosphorothioate internucleotide linkages on the PBS.

In some embodiments, the modified phosphate group comprises exactly three phosphorothioate internucleotide linkages on the PBS.

In some embodiments, the modification of the nucleobase group is selected from 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups.

In some embodiments, said petRNA comprises one MS2 hairpin.

In some embodiments, said petRNA comprises two MS2 hairpins.

In some embodiments, said petRNA comprises three MS2 hairpins.

In some embodiments, said petRNA comprises four MS2 hairpins.

In some embodiments, the at least one MS2 hairpin is chemically modified.

In some embodiments, the one or more modified nucleotides of the MS2 hairpin comprises a modification of a ribose group, a phosphate group, a nucleobase, or a combination thereof.

In some embodiments, the modified MS2 hairpin comprises a phosphate group comprising a phosphorothioate, phosphonoacetate (PACE), thiophosphonoacetate (thioPACE), amide, triazole, phosphonate, or phosphotriester modification.

In some embodiments, the modified MS2 hairpin comprises a phosphate group comprising at least one phosphorothioate internucleotide linkage.

In some embodiments, the modified MS2 hairpin comprises a phosphate group comprising three phosphorothioate internucleotide linkages.

In some embodiments, the modified MS2 hairpin comprises a phosphate group comprising ten phosphorothioate internucleotide linkages.

In some embodiments, the modified MS2 hairpin comprises a phosphate group comprising twenty-three phosphorothioate internucleotide linkages.

In some embodiments, the phosphorothioate internucleotide linkages are located on the N terminus.

In some embodiments, the phosphorothioate internucleotide linkages are located on the C terminus.

In some embodiments, the modification of the nucleobase group is selected from 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups.

In some embodiments, said petRNA comprises a fully modified MS2 hairpin.

In some embodiments, the MS2 is linked to the RTT using a linker.

In some embodiments, the linker is selected from the group consisting of ethylene glycol and polyethylene glycol (PEG).

In some embodiments, the PEG is a hexaethylene glycol (HEX).

In some embodiments, HEX comprises the following structure:

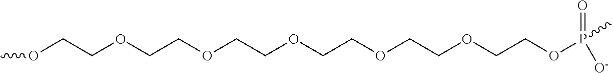

In some embodiments, the PEG is 2×HEX.

In some embodiments, the PEG is 2×HEX comprising the following structure:

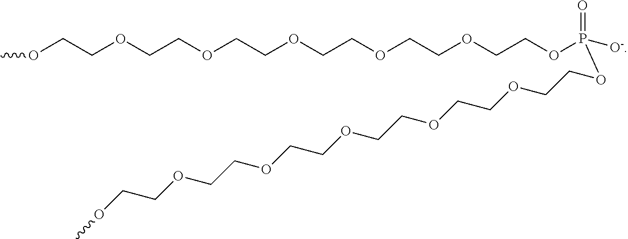

In some embodiments, the linker is a 2′-Omethyl modified RNA.

In some embodiments, the 2′-Omethyl modified RNA consists of A and N nucleotide residues.

In some embodiments, the 2′-Omethyl modified RNA is between 1 and 15 nucleotides long.

In some embodiments, the 2′-Omethyl modified RNA is 5 nucleotides long.

In some embodiments, the 2′-Omethyl modified RNA is 10 nucleotides long.

In some embodiments, the 2′-Omethyl modified RNA comprises the following sequence from the N-terminus to the C-terminus: AAACACA.

In certain aspects, provided herein is a petRNA a comprising a primer binding site, a nucleotide polymerase template (NPT), and at least one MS2 hairpin, wherein the MS2 is linked to the RTT using a linker.

In some embodiments, the MS2 is linked to the RTT using a linker.

In some embodiments, the linker is selected from the group consisting of ethylene glycol and polyethylene glycol (PEG).

In some embodiments, the PEG is a hexaethylene glycol (HEX) with the following structure:

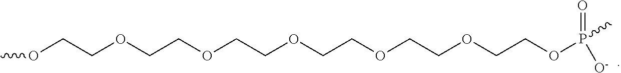

In some embodiments, the PEG is a 2×HEX.

In some embodiments, the PEG is a 2×HEX with the following structure:

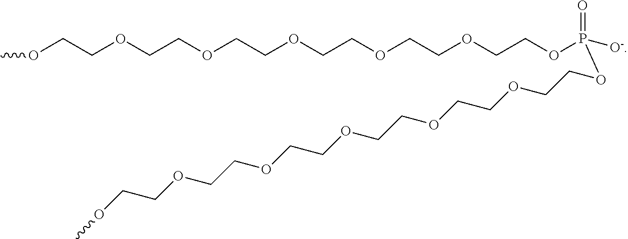

In some embodiments, the linker is a 2′-Omethyl modified RNA.

In some embodiments, the 2′-Omethyl modified RNA consists of A and N nucleotide residues.

In some embodiments, the 2′-Omethyl modified RNA is between 1 and 15 nucleotides long.

In some embodiments, the 2′-Omethyl modified RNA is 5 nucleotides long.

In some embodiments, the 2′-Omethyl modified RNA is 10 nucleotides long.

In some embodiments, the 2′-Omethyl modified RNA comprises the following sequence from the N-terminus to the C-terminus: AAACACA.

### BRIEF DESCRIPTION OF THE DRAWINGS

FIGS. 1A-B shows an exemplary embodiment of a PE system comprising a split effector and an exemplary embodiment of an sPE system comprising a split guide RNA (gRNA). The diagram in FIG. 1A illustrates an exemplary split effector Prime Editor (sPE) system comprising an untethered nCas9 and an NP template. The diagram in FIG. 1B illustrates a split petRNA comprises an untethered single guide RNA (sgRNA) and a prime editor template RNA (petRNA) molecule, an RNA molecule that encodes a primer binding site (PBS), a nucleotide polymerase template (NPT), and a stem loop (MS2 stem loop).

FIGS. 2A-C show the different effector formats and their precise editing efficiencies in mCherry cells. FIG. 2A shows an illustrative diagram of several PE variants including split effectors (sPE), MS2 coat protein (MCP) fused PE (mM-PE), C-terminal MCP-fused PE (cM-PE), N-terminal MCP-fused PE (nM-PE), N-terminal MCP-dimer fused PE (nMM-PE), N-terminal and C-terminal MCP-fused PE (nMcM-PE) and their abbreviations. FIGS. 2B and C show a comparison of 7 prime editor constructs for their ability to install a +1 AGAC sequence insert (FIG. 2B) or to replace a 39 bp sequence by an 18 bp sequence (FIG. 2C) without a nicking sgRNA in a “traffic light reporter” (TLR-MCV1) locus in HEK-293T cells. Editing efficiencies reflect mCherry positive cells quantified using flow cytometry. Values and error bars reflect mean±s.d. of n=3 independent biological replicates.

FIGS. 3A-C show the petRNA-based PE efficiency and indel generation by nMM-PE of 3 different loci: EXM1 (FIG. 3A), HEXA (FIG. 3B), and IDUA (FIG. 3C) in human HEK-293T cells. A G·C-to-T·A transversion editing efficiency at the +5 position of EMX1 using petRNA is shown in FIG. 3A, a +1 TATC insert at the HEXA site using petRNA is shown in FIG. 3B, and a +5 G to A edit using a petRNA plasmid is shown in FIG. 3C. Editing efficiencies reflect perfect of sequencing reads that contain the intended edit and do not contain indels among all treated cells. Indels are also indicated using the bar on the right.

FIGS. 4A-D show the petRNA-based PE efficiency and indel generation by nMM-PE of 4 different loci: HBB (FIG. 4A), VEGFA (FIG. 4B), RUNX1 (FIG. 4C), and PSEN1 (FIG. 4D) in human HEK-293T cells. A+4-5 A·G-to-T·A edit in HBB using a petRNA plasmid is shown in FIG. 4A, a +2 G to C and a +4-5 G·G-to-C·T edit in VEGFA using a petRNA plasmid is shown in FIG. 4B, a +5 G to T edit in RUNX1 using a petRNA plasmid is shown in FIG. 4C, and a +5 G to T edit in PSEN1 using a petRNA plasmid is shown in FIG. 4D. Editing efficiencies reflect sequencing reads that contain the intended edit and do not contain indels among all treated cells. Indels are also indicated using the bar on the right.

FIGS. 5A-5D show the petRNA-based PE efficiency and indel generation by nMM-PE of 4 different loci: IDS (FIG. 5A), FANCF (FIG. 5B), PRNP (FIG. 5C), and DNMT1 (FIG. 5D) in human HEK-293T cells. A+5 G to Ax edit in IDS using a petRNA plasmid is shown in FIG. 5A, a +2 G to C and a +4-6 G·G-to-C·T edit in FANCF using a petRNA plasmid is shown in FIG. 5B, a +6 G to T edit in PRNP using a petRNA plasmid is shown in FIG. 5C, and a +6 G to T edit in DNMT1 using a petRNA plasmid is shown in FIG. 5D. Editing efficiencies reflect sequencing reads that contain the intended edit and do not contain indels among all treated cells. Indels are also indicated using the bar on the right.

FIG. 6 shows the petRNA-based PE efficiency and indel generation by nMM-PE as compared to canonical pegRNA-based editing in 11 endogenous loci in HEK-293T cells. Editing efficiencies reflect sequencing reads that contain the intended edit and do not contain indels among all treated cells. Indels are also indicated using the bar on the right.

FIGS. 7A-C show the different domain inlaid base editors and their precise editing efficiencies in mCherry cells. FIG. 7A shows an illustrative diagram of several PE variants including the MCP inlaid PE as compared to the conical PE (no MCP), the sPE (MCP between the Cas9 H840A nickase and the RT), the nMM-PE with (MCP dimer placed by the Cas9 H840A nickase), and the MCP inlaid PE (MCP dimer placed in the middle of the H840A nickase at several positions: iM-S355-PE, iMM-E1026-PE, iMM-N1054-PE, iMM-G1247-PE. iMM-D1299-PE. iMM-E827-PE. And iMM-delta (S793-R905)-PE), and variants' abbreviations. MCP inlaid positions include Rec-I (355), RuvC-III (1026, 1054), PID (1247, 1299), and HNH [827, delta (792-905)]. FIGS. 7B and C show a comparison of 7 MCP inlaid PE systems with the conical PE, sPE, and the nMM-PE for their ability to install a +1 AGAC sequence insert (FIG. 7B) or to replace a 39 bp sequence by an 18 bp sequence (FIG. 7C) without a nicking sgRNA in a TLR-MCV1 locus in HEK-293T cells. Editing efficiencies reflect mCherry positive cells quantified using flow cytometry. Values and error bars reflect mean±s.d. of n=3 independent biological replicates.

FIG. 8 shows an illustrative diagram of several PE variants with MCP dimers or multimers inserted at one or more positions. The illustration shows a conical PE (containing a Cas9 H840A nickase and an RT), an sPE (containing an MCP monomer between the Cas9 H840A nickase and the RT), a nMM-PE (N-terminal MCP dimer fused PE), an iMM-G1247-PE (MCP dimer inserted in inlaid position G1247 of the Cas9 H840A nickase), a nMMMM-PE (N-terminal MCP tetramer-fused PE), an nMMcMM-PE, (contains both an N-terminal and a C-terminal MCP dimer), and an nMM-iMM-G1247-PE (contains both an N-terminal MCP dimer and an MCP dimer inserted in the inlaid position G1247 of the Cas9 H840A nickase).

FIGS. 9A-B show the editing efficiencies of the different PE variants containing MCP dimers or multimers shown in FIGS. 10A-C in mCherry cells. FIGS. 9A and B show a comparison of the four PE variants with MCP dimers and monomers at several positions with the conical PE, sPE, and the nMM-PE for their ability to install a +1 AGAC sequence insert (FIG. 9A) or to replace a 39 bp sequence by an 18 bp sequence (FIG. 9B) without a nicking sgRNA in a TLR-MCV1 locus in HEK-293T cells. Editing efficiencies reflect mCherry positive cells quantified using flow cytometry. Values and error bars reflect mean±s.d. of n=3 independent biological replicates.

FIGS. 10A-C show the pegRNA-based PE efficiency and indel generation by the four PE variants with MCP dimers and monomers at several positions with the conical PE, sPE, and the nMM-PE of 3 different loci: EXM1 (FIG. 10A), HEXA (FIG. 10B), and IDUA (FIG. 10C) in human HEK-293T cells. A G·C-to-T·A transversion editing efficiency at the +5 position of EMX1 using the pegRNA variants is shown in FIG. 10A, a +1 TATC insert in HEXA using petRNA is shown in FIG. 10B, and a +5 G to A edit in IDUA is shown in FIG. 10C. Editing efficiencies reflect perfect of sequencing reads that contain the intended edit and do not contain indels among all treated cells. Indels are also indicated using the bar on the right.

FIGS. 11A-I show the pegRNA-based PE efficiency and indel generation by the four PE variants with MCP dimers and monomers at several positions with the conical PE, sPE, and the nMM-PE of 8 different loci: HBB (FIG. 11A), VEGFA (FIG. 11B), RUNX1 (FIG. 11C), PSEN1 (FIGS. 11D and 11I), IDS (FIG. 11E), FANCF (FIG. 11F), PRNP (FIG. 11G), and DNMT1 (FIG. 11H) in human HEK-293T cells. A+4-5 A·G-to-T·A edit in HBB using the pegRNA variants delivered using a plasmid is shown in FIG. 11A, a +2 G to C and a +4-5 G·G-to-C·T edit in VEGFA is shown in FIG. 11B, a +5 G to T edit in RUNX1 is shown in FIG. 11C, a +5 G to T edit in PSEN1 is shown in FIG. 11D, A+5 G to Ax edit in IDS is shown in FIG. 11E, a +2 G to C a +4-6 G·G-to-C·T edit in FANCF is shown in FIG. 11F, a +6 G to T edit in PRNP is shown in FIG. 11G, a +6 G to T edit in DNMT1 is shown in FIG. 11H, and a +6 G to A edit in PSEN1 is shown in FIG. 11I. Editing efficiencies reflect sequencing reads that contain the intended edit and do not contain indels among all treated cells. Indels are also indicated using the bar on the right.

FIG. 12 shows the summary of the editing efficiencies and indel generation by the four PE variants with MCP dimers and monomers at several positions as compared the conical PE, sPE, and the nMM-PE of 11 endogenous loci delivered using a plasmid in human HEK-293T cells. Editing efficiencies reflect sequencing reads that contain the intended edit and do not contain indels among all treated cells. Indels are also indicated using white bars.

FIG. 13 shows a comparison of the peg PE, sPE, and petRNA-based PE efficiency of several constructs containing different linkers for their ability to install a +1 AGAC sequence insert in a TLR-MCV1 locus in HEK-293T cells. These constructs were delivered as an mRNA and as a synthetic petRNA (L-Pet). Linkers included either adding a 3 OMe, a 10 OMe, or a 23 OMe at the MS2 stem loop or adding no linker, an AC7 (or OMe), or a 2×HEG linker between the MS2 loop and the NPT. Editing efficiencies reflect mCherry positive cells quantified using flow cytometry.

### DETAILED DESCRIPTION OF THE DISCLOSURE

The present invention relates to the field of genomic engineering. In particular, a modular prime editing (sPE) system is disclosed comprising components including, but not limited to, a fusion protein comprising a Cas9 nickase (nCas9) protein and a transcriptase protein, a petRNA comprising a primer binding site (PBS), a nucleotide polymerase template (NPT), and at least one MS2 hairpin, and a single guide ribonucleic acid (sgRNA), such that both the fusion protein comprises an nCas9 and an NP protein that are linked, and such as the petRNA comprising the PBS, the NPT and the at least one MS2 binding protein and the sgRNA are free and independent molecules. This modular sPE composition results in precise and efficient genome editing in cells and in adult mouse liver which is advantageous over conventional split PE fusion constructs. Furthermore, the prime editing efficiencies of several constructs were tested, these included: 1) a prime editor with one or more MCP at several different orientations, and 2) a pegRNA with one or more MS2 stem loops were tested. These included PE systems that comprise an MCP dimer inlaid within the nCas9 sequence of SEQ ID NO: 1 and PE systems with an MCP dimer at the 5′ orientation of the fusion protein. These constructs resulted in a surprising precise and efficient genome editing in cells which is advantageous over conventional sgRNA prime editor fusion constructs. This flexible, and modular system is an improvement in the art to obtain precise genome editing.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “catalytically impaired Cas9 nickase” or “nCas9”, as used herein refers to a mutated Cas9 which renders the nuclease able to cleave only one strand of deoxyribonucleic acid backbone. Depending on the position of the mutation within the Cas9 protein sequence either the target or non-target strand is cleaved. In the case of a prime editor the non-target strand is selectively cleaved.

The term “engineered reverse transcriptase” as used herein, refers to a protein that converts RNA into DNA and contains specific mutations that effect its activity efficiency. One example of a reverse transcriptase is a Moloney murine leukemia virus reverse transcriptase (M-MLV RT).

The term “reverse transcriptase template” as used herein refers to a ribonucleic acid sequence that is utilized as a substrate for a reverse transcriptase protein that is part of the fusion protein complex as contemplated herein. Such templates provide the necessary information to edit a DNA sequence to support conversions including, but not limited to, base conversions, sequence insertions or sequence deletions.

The term “nucleotide polymerase template” or “NPT” as used herein refers to a deoxyribonucleic or a ribonucleic acid sequence and modifications thereof, that is utilized as a nucleic acid for a nucleotide polymerase protein (e.g., RNA polymerase or DNA polymerase) that is part of the chimeric prime editor complex as contemplated herein. Such templates provide the necessary information to edit a DNA sequence to support conversions including, but not limited to, base conversions, sequence insertions or sequence deletions.

The term “primer binding site” as used herein, refers to a specific nucleic acid sequence within the pegRNA or the petRNA that is complementary to the 3′ end of the nicked DNA strand. This allows annealing of the free 3′ end of the genomic DNA for extension by the nucleotide polymerase based on the template sequence encoded in the pegRNA or the petRNA.

The term, “prime editing guide RNA molecule” or “pegRNA molecule” as used herein, refers to a Cas9 guide RNA molecule that encodes the crRNA-tracrRNA fused to a primer binding site (PBS) and a nucleotide polymerase template (NPT) nucleic acid sequence. The primer binding site hybridizes to a desired genomic sequence released by the binding and cleavage of the Cas9 nickase. The 3′ end of the genomic sequence is extended by the nucleotide polymerase based on the nucleotide polymerase template sequence.

The term, “prime editor template RNA” or “petRNA molecule” as used herein, refers to an RNA molecule that encodes a primer binding site (PBS) and a nucleotide polymerase template (NPT). The petRNA may also encode stem loops. The petRNA may also be linear or circularized. Unlike the pegRNA, the petRNA does not include the guide RNA component.

The term “editing” or “gene editing” as used herein, refers to a genetic manipulation of a DNA sequence. Such a manipulation includes, but is not limited to, a base conversion, a sequence insertion and/or a sequence deletion. The term “group I catalytic intron” as used herein, refers to large self-splicing ribozymes which self-catalyze an excision from ribonucleotides including, but not limited to, mRNA, tRNA and rRNA. See, FIG. 19. Nielsen et al., “Group I introns: Moving in new directions” RNA Biol. 6 (4): 375-83 (2009); and Cech T., “Self-splicing of group I introns” Annu. Rev. Biochem. 59:543-568 (1990). Their core secondary structure included paired regions. Woodson S, “Structure and assembly of group I introns” Curr. Opin. Struct. Biol. 15 (3): 324-330 (2005). These paired regions self-assembly into domains: i) the P4-P6 domain formed from stacking of P5, P4, P6 and P6a helices; and ii) the P3-P9 domain formed from the P8, P3, P7 and P9 helices). Cate et al., “Crystal structure of a group I ribozyme domain: principles of RNA packing”. Science. 273 (5282): 1678-1685 (1996). Group I introns often have long open reading frames inserted in loop regions.

The term “prime editing” as used herein, is a genome editing technology by which the genome of living organisms may be modified. Prime editing manipulates the genetic information of a targeted DNA site to essentially “rewrite” the coded sequences.

The term “prime editor” or “PE” as used herein, is a fusion protein comprising a catalytically impaired Cas9 endonuclease that can nick DNA and is fused to an engineered nucleotide polymerase enzyme. The petRNA comprising a PBS, an NPT along with a single guide RNA (sgRNA), are capable of programming the nCas9 to recognize a target site with the encoded crRNA-tracrRNA (as does a conventional single guide RNA). The resulting nicked genomic DNA can be extended by the nucleotide polymerase based on the petRNA template sequence to contain a new sequence. Once one strand is recoded, cellular DNA repair pathways can cause conversion of the local DNA sequence to match the new sequence. Such manipulation includes, but is not limited to, insertions, deletions, and base-to-base conversions without the need for double strand breaks (DSBs) or donor DNA templates. For example, such prime editing may be performed by a Cas9 CRISPR platform programmed with a petRNA and an sgRNA, such as a catalytically impaired Cas9 nickase platform with an appropriate nucleotide polymerase.

The term “conversion” as used herein, refers to any manipulation of a nucleic acid sequence that converts a mutated sequence into a wildtype sequence, or a wildtype sequence into a mutated sequence. For example, a converted sequence includes, but is not limited to, a base pair conversion, a nucleic acid sequence insertion or a nucleic acid sequence deletion. The term “editing-related indels” as used herein, refers to the generation of off-target and/or unintended nucleotide sequence insertions created by a prime editor.

The term “split-intein prime editor protein” refers to a prime editor protein that has been split into amino-terminal (PE2-N) and carboxy-terminal (PE2-C) segments, which are then fused into a full length PE by a trans-splicing intein. This configuration imparts flexibility to the prime editor thereby facilitating a packaging into an adeno-associated virus (AAV).

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer” sequence. The spacers are short segments of DNA from a virus and may serve as a ‘memory’ of past exposures to facilitate an adaptive defense against future invasions. Doudna et al. Genome editing. The new frontier of genome engineering with CRISPR-Cas9″ Science 346 (6213): 1258096 (2014).

As used herein, the term “Cas” or “CRISPR-associated (cas)” refers to genes often associated with CRISPR repeat-spacer arrays.

As used herein, the term “Cas9” refers to a nuclease from type II CRISPR systems, an enzyme specialized for generating double-strand breaks in DNA, with two active cutting sites (the HNH and RuvC domains), one for each strand of the double helix. tracrRNA and spacer RNA may be combined into a “single-guide RNA” (sgRNA) molecule that, mixed with Cas9, could find and cleave DNA targets through Watson-Crick pairing between the guide sequence within the sgRNA and the target DNA sequence, Jinek et al. A programmable dual-RNA-guided DNA endonuclease in adaptive bacterial immunity” Science 337 (6096): 816-821 (2012).

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact. Jinek et al., “A programmable dual-RNA-guided DNA endonuclease in adaptive bacterial immunity” Science 337 (6096): 816-821 (2012) and Cong et al. Multiplex genome engineering using CRISPR/Cas systems” Science 339 (6121): 819-823 (2013). The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into construct complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to the repeat sequence of the pre-crRNA, forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “protospacer adjacent motif” or “PAM” as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

The terms “protospacer adjacent motif recognition domain”, “PAM Interacting Domain” or “PID” as used herein, refers to a Cas9 amino acid sequence that comprises a binding site to a DNA target PAM sequence.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinck et ak, “A programmable dual-RNA-guided DNA endonuclease in adaptive bacterial immunity” Science 337 (6096): 816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage. Esvelt et al., “Orthogonal Cas9 proteins for RNA-guided gene regulation and editing” Nat Methods 10 (11): 1116-1121 (2013). For example, this would allow one Cas9 isoform (e.g. S. pyogenes Cas9 or SpyCas9) to function as a nuclease programmed by a sgRNA that may be specific to it, and another Cas9 isoform (e.g. N meningitidis Cas9 or NmeCas9) to operate as a nuclease-dead Cas9 that provides DNA targeting to a binding site through its PAM specificity and orthogonal sgRNA. Other Cas9s include S. aureus Cas9 or SauCas9 and A. naeslundii Cas9 or AnaCas9.

The term “truncated” as used herein, when used in reference to either a polynucleotide sequence or an amino acid sequence means that at least a portion of the wild type sequence may be absent. In some cases, truncated guide sequences within the sgRNA or crRNA may improve the editing precision of Cas9. Fu, et al. “Improving CRISPR-Cas nuclease specificity using truncated guide RNAs” Nat Biotechnol. 2014 March; 32 (3): 279-284 (2014).

The term “base pairs” as used herein, refer to specific nucleobases (also termed nitrogenous bases), that are the building blocks of nucleotide sequences that form a primary structure of both DNA and RNA. Double-stranded DNA may be characterized by specific hydrogen bonding patterns. Base pairs may include, but are not limited to, guanine-cytosine and adenine-thymine base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence”, as used herein, refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

### RNA-Guided Nucleases

RNA-guided nucleases according to the present disclosure include, without limitation, naturally occurring Type II CRISPR nucleases such as Cas9, as well as other nucleases derived or obtained therefrom. Exemplary Cas9 nucleases that may be used in the present disclosure include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally occurring PAM specificity vs. engineered PAM specificity).

Various RNA-guided nucleases may require different sequential relationships between PAMs and protospacers. In general, Cas9s recognize PAM sequences that are 5′ of the protospacer as visualized relative to the top or complementary strand.

In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are described below.

RNA-guided nucleases are also characterized by their DNA cleavage activity: naturally occurring RNA-guided nucleases typically form DSBs in target nucleic acids, but engineered variants have been produced that generate only SSBs (discussed above; see also Ran 2013, incorporated by reference herein), or that do not cut at all.

The RNA-guided nuclease Cas9 may be a variant of Cas9 with altered activity. Exemplary variant Cas9 nucleases include, but are not limited to, a Cas9 nickase (nCas9, Table 1), a catalytically dead Cas9 (dCas9), a hyper accurate Cas9 (HypaCas9) (Chen et al. Nature, 550 (7676), 407-410 (2017)), a high fidelity Cas9 (Cas9-HF) (Kleinstiver et al. Nature 529 (7587), 490-495 (2016)), an enhanced specificity Cas9 (eCas9) (Slaymaker et al. Science 351 (6268), 84-88 (2016)), and an expanded PAM Cas9 (xCas9) (Hu et al. Nature doi: 10.1038/nature26155 (2018)).

TABLE 1
SpyCas9 H840A Nickase Sequence

| | |
|---|---|
| SpyCas9 H840A | DKKYSIGLDIGTNSVGWAVITDEYKVPSKKFKVLGNTDRHSIK |
| nickase | KNLIGALLFDSGETAEATRLKRTARRRYTRRKNRICYLQEIFSN |
| (SEQ ID NO: 1) | EMAKVDDSFFHRLEESFLVEEDKKHERHPIFGNIVDEVAYHEK |
| | YPTIYHLRKKLVDSTDKADLRLIYLALAHMIKFRGHFLIEGDL |
| | NPDNSDVDKLFIQLVQTYNQLFEENPINASGVDAKAILSARLS |
| | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNF |
| | MQLIHDDSLTFKEDIQKAQVSGQGDSLHEHIANLAGSPAIKKG |
| | ILQTVKVVDELVKVMGRHKPENIVIEMARENQTTQKGQKNSR |
| | ERMKRIEEGIKELGSQILKEHPVENTQLQNEKLYLLQNGRDM |
| | YVDQELDINRLSDYDVDAIVPQSFLKDDSIDNKVLTRSDKNRG |
| | KSDNVPSEEVVKKMKNYWRQLLNAKLITQRKFDNLTKAERG |
| | GLSELDKAGFIKRQLVETRQITKHVAQILDSRMNTKYDENDK |
| | LIREVKVITLKSKLVSDFRKDFQFYKVREINNYHHAHDAYLNA |
| | VVGTALIKKYPKLESEFVYGDYKVYDVRKMIAKSEQEIGKAT |
| | AKYFFYSNIMNFFKTEITLANGEIRKRPLIETNGETGEIVWDKG |
| | RDFATVRKVLSMPQVNIVKKTEVQTGGFSKESILPKRNSDKLI |
| | ARKKDWDPKKYGGFDSPTVAYSVLVVAKVEKGKSKKLKSV |
| | KELLGITIMERSSFEKNPIDFLEAKGYKEVKKDLIIKLPKYSLFE |
| | LENGRKRMLASAGELQKGNELALPSKYVNFLYLASHYEKLK |
| | GSPEDNEQKQLFVEQHKHYLDEIIEQISEFSKRVILADANLDKV |
| | LSAYNKHRDKPIREQAENIIHLFTLTNLGAPAAFKYFDTTIDRK |
| | RYTSTKEVLDATLIHQSITGLYETRIDLSQLGGD |

The RNA-guided nucleases may be combined with the chemically modified guide RNAs of the present disclosure to form a genome-editing system. The RNA-guided nucleases may be combined with the chemically modified guide RNAs to form an RNP complex that may be delivered to a cell where genome-editing is desired. The RNA-guided nucleases may be expressed in a cell where genome-editing is desired with the chemically modified guide RNAs delivered separately. For example, the RNA-guided nucleases may be expressed from a polynucleotide such as a vector or a synthetic mRNA. The vector may be a viral vector, including, be not limited to, an adeno-associated virus (AAV) vector or a lentivirus (LV) vector. A Cas9 fusion polypeptide (Cas9 fusion protein) may have multiple (1 or more, 2 or more, 3 or more, 4 or more, 5 or more, 6 or more, etc.) fusion partners in any combination. As an illustrative example, a Cas9 fusion protein can have a heterologous sequence that provides an activity (e.g., for transcription modulation such as a nucleotide polymerase protein, target modification, modification of a protein associated with a target nucleic acid, etc.) and can also have a subcellular localization sequence (e.g., 1 or more NLSs, Table 2).

TABLE 2
Sequences of the NLS protein and other tags

| | |
|---|---|
| Bipartite SV40 NLS | MKRTADGSEFESPKKKRKV |
| attached on the 5′ | |
| of the fusion | |
| protein (SEQ ID NO: | |
| 22) | |
| Bipartite SV40 NLS | KRTADGSEFEPKKKRKV |
| attached on the 3′ | |
| of the fusion | |
| protein (SEQ ID NO: | |
| 23) | |
| SV40 NLS (SEQ ID NO: 24) | PKKKRKV |
| Nucleoplasmin NLS | KRPAATKKAGQAKKKK |
| (SEQ ID NO: 25) | |
| 3XFLAG (SEQ ID NO: 53) | MDYKDHDGDYKDHDIDYKDDDDK |

In some cases, such a Cas9 fusion protein might also have a tag for ease of tracking and/or purification (e.g., green fluorescent protein (GFP), YFP, RFP, CFP, mCherry, tdTomato, and the like; a histidine tag, e.g., a 6×His (SEQ ID NO:112) tag; a hemagglutinin (HA) tag; a FLAG tag; a Myc tag; and the like). As another illustrative example, a Cas9 protein can have one or more NLSs (e.g., two or more, three or more, four or more, five or more, 1, 2, 3, 4, or 5 NLSs). In some cases, a fusion partner (or multiple fusion partners) (e.g., an NLS, a tag, a fusion partner providing an activity, etc.) is located at or near the C-terminus of Cas9. In some cases, a fusion partner (or multiple fusion partners) (e.g., an NLS, a tag, a fusion partner providing an activity, etc.) is located at the N-terminus of Cas9. In some cases, a Cas9 has a fusion partner (or multiple fusion partners) (e.g., an NLS, a tag, a fusion partner providing an activity, etc.) at both the N-terminus and C-terminus.

As used herein, the term “inlaid” refers to a first protein domain (e.g., an RT domain or MS2 binding protein) that is inserted between two amino acids of a second protein domain (e.g., a Cas9 protein domain).

TABLE 3
Sequences of the N-terminus portion of the SpyCas9 H840A Nickase

| | |
|---|---|
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| iM-S355-PE | YPTIYHLRKKLVDSTDKADLRLIYLALAHMIKFRGHFLIEGDL |
| (SEQ ID NO: 2) | NPDNSDVDKLFIQLVQTYNQLFEENPINASGVDAKAILSARLS |
| | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQS |
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| iMM-E1026-PE | YPTIYHLRKKLVDSTDKADLRLIYLALAHMIKFRGHFLIEGDL |
| (SEQ ID NO: 3) | NPDNSDVDKLFIQLVQTYNQLFEENPINASGVDAKAILSARLS |
| | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNFM |
| | QLIHDDSLTFKEDIQKAQVSGQGDSLHEHIANLAGSPAIKKGIL |
| | QTVKVVDELVKVMGRHKPENIVIEMARENQTTQKGQKNSRE |
| | RMKRIEEGIKELGSQILKEHPVENTQLQNEKLYLLONGRDMY |
| | VDQELDINRLSDYDVDAIVPQSFLKDDSIDNKVLTRSDKNRGK |
| | SDNVPSEEVVKKMKNYWRQLLNAKLITQRKFDNLTKAERGG |
| | LSELDKAGFIKRQLVETRQITKHVAQILDSRMNTKYDENDKLI |
| | REVKVITLKSKLVSDFRKDFQFYKVREINNYHHAHDAYLNAV |
| | VGTALIKKYPKLESEFVYGDYKVYDVRKMIAKSE |
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| iMM-N1054-PE | YPTIYHLRKKLVDSTDKADLRLIYLALAHMIKFRGHFLIEGDL |
| (SEQ ID NO: 4) | NPDNSDVDKLFIQLVQTYNQLFEENPINASGVDAKAILSARLS |
| | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNFM |
| | QLIHDDSLTFKEDIQKAQVSGQGDSLHEHIANLAGSPAIKKGIL |
| | QTVKVVDELVKVMGRHKPENIVIEMARENQTTQKGQKNSRE |
| | RMKRIEEGIKELGSQILKEHPVENTQLQNEKLYLLQNGRDMY |
| | VDQELDINRLSDYDVDAIVPQSFLKDDSIDNKVLTRSDKNRGK |
| | SDNVPSEEVVKKMKNYWRQLLNAKLITQRKFDNLTKAERGG |
| | LSELDKAGFIKRQLVETRQITKHVAQILDSRMNTKYDENDKLI |
| | REVKVITLKSKLVSDFRKDFQFYKVREINNYHHAHDAYLNAV |
| | VGTALIKKYPKLESEFVYGDYKVYDVRKMIAKSEQEIGKATA |
| | KYFFYSNIMNFFKTEITLAN |
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| iMM-G1247-PE | YPTIYHLRKKLVDSTDKADLRLIYLALAHMIKFRGHFLIEGDL |
| and nMM-iMM- | NPDNSDVDKLFIQLVQTYNQLFEENPINASGVDAKAILSARLS |
| G1247-PE | KSRRLENLIAQLPGEKKNGLFGNLIALSLGLTPNFKSNFDLAED |
| (SEQ ID NO: 5) | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNFM |
| | QLIHDDSLTFKEDIQKAQVSGQGDSLHEHIANLAGSPAIKKGIL |
| | QTVKVVDELVKVMGRHKPENIVIEMARENQTTQKGQKNSRE |
| | RMKRIEEGIKELGSQILKEHPVENTQLQNEKLYLLQNGRDMY |
| | VDQELDINRLSDYDVDAIVPQSFLKDDSIDNKVLTRSDKNRGK |
| | SDNVPSEEVVKKMKNYWRQLLNAKLITQRKFDNLTKAERGG |
| | LSELDKAGFIKRQLVETRQITKHVAQILDSRMNTKYDENDKLI |
| | REVKVITLKSKLVSDFRKDFQFYKVREINNYHHAHDAYLNAV |
| | VGTALIKKYPKLESEFVYGDYKVYDVRKMIAKSEQEIGKATA |
| | KYFFYSNIMNFFKTEITLANGEIRKRPLIETNGETGEIVWDKGR |
| | DFATVRKVLSMPQVNIVKKTEVQTGGFSKESILPKRNSDKLIA |
| | RKKDWDPKKYGGFDSPTVAYSVLVVAKVEKGKSKKLKSVKE |
| | LLGITIMERSSFEKNPIDFLEAKGYKEVKKDLIIKLPKYSLFELE |
| | NGRKRMLASAGELQKGNELALPSKYVNFLYLASHYEKLKG |
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| iMM-D1299-PE | YPTIYHLRKKLVDSTDKADLRLIYLALAHMIKFRGHFLIEGDL |
| (SEQ ID NO: 6) | NPDNSDVDKLFIQLVQTYNQLFEENPINASGVDAKAILSARLS |
| | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNFM |
| | QLIHDDSLTFKEDIQKAQVSGQGDSLHEHIANLAGSPAIKKGIL |
| | QTVKVVDELVKVMGRHKPENIVIEMARENQTTQKGQKNSRE |
| | RMKRIEEGIKELGSQILKEHPVENTQLQNEKLYLLONGRDMY |
| | VDQELDINRLSDYDVDAIVPQSFLKDDSIDNKVLTRSDKNRGK |
| | SDNVPSEEVVKKMKNYWRQLLNAKLITQRKFDNLTKAERGG |
| | LSELDKAGFIKRQLVETRQITKHVAQILDSRMNTKYDENDKLI |
| | REVKVITLKSKLVSDFRKDFQFYKVREINNYHHAHDAYLNAV |
| | VGTALIKKYPKLESEFVYGDYKVYDVRKMIAKSEQEIGKATA |
| | KYFFYSNIMNFFKTEITLANGEIRKRPLIETNGETGEIVWDKGR |
| | DFATVRKVLSMPQVNIVKKTEVQTGGFSKESILPKRNSDKLIA |
| | RKKDWDPKKYGGFDSPTVAYSVLVVAKVEKGKSKKLKSVKE |
| | LLGITIMERSSFEKNPIDFLEAKGYKEVKKDLIIKLPKYSLFELE |
| | NGRKRMLASAGELQKGNELALPSKYVNFLYLASHYEKLKGSP |
| | EDNEQKQLFVEQHKHYLDEIIEQISEFSKRVILADANLDKVLSA |
| | YNKHRD |
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| iMM-E827-PE | YPTIYHLRKKLVDSTDKADLRLIYLALAHMIKFRGHFLIEGDL |
| (SEQ ID NO: 7) | NPDNSDVDKLFIQLVQTYNQLFEENPINASGVDAKAILSARLS |
| | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNFM |
| | QLIHDDSLTFKEDIQKAQVSGQGDSLHEHIANLAGSPAIKKGIL |
| | QTVKVVDELVKVMGRHKPENIVIEMARENQTTQKGQKNSRE |
| | RMKRIEEGIKELGSQILKEHPVENTQLQNEKLYLLQNGRDMY |
| | VDQE |
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| iMM-delta(S793- | YPTIYHLRKKLVDSTDKADLRLIYLALAHMIKFRGHFLIEGDL |
| R905)-PE | NPDNSDVDKLFIQLVQTYNQLFEENPINASGVDAKAILSARLS |
| (SEQ ID NO: 8) | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNFM |
| | QLIHDDSLTFKEDIQKAQVSGQGDSLHEHIANLAGSPAIKKGIL |
| | QTVKVVDELVKVMGRHKPENIVIEMARENQTTQKGQKNSRE |
| | RMKRIEEGIKELG |
| N-terminus | DKKYSIGLDIGTNSVGWAVITDEYKVPSKKFKVLGNTDRHSIK |
| portion of the | KNLIGALLFDSGETAEATRLKRTARRRYTRRKNRICYLQEIFSN |
| Cas9 nickase of | EMAKVDDSFFHRLEESFLVEEDKKHERHPIFGNIVDEVAYHEK |
| nMM-iMM- | YPTIYHLRKKLVDSTDKADLRLIYLALAHMIKFRGHFLIEGDL |
| G1247-PE | NPDNSDVDKLFIQLVQTYNQLFEENPINASGVDAKAILSARLS |
| (SEQ ID NO: 9) | KSRRLENLIAQLPGEKKNGLFGNLIALSLGLTPNFKSNFDLAED |
| | AKLQLSKDTYDDDLDNLLAQIGDQYADLFLAAKNLSDAILLS |
| | DILRVNTEITKAPLSASMIKRYDEHHQDLTLLKALVRQQLPEK |
| | YKEIFFDQSKNGYAGYIDGGASQEEFYKFIKPILEKMDGTEELL |
| | VKLNREDLLRKQRTFDNGSIPHQIHLGELHAILRRQEDFYPFLK |
| | DNREKIEKILTFRIPVGPLARGNSRFAWMTRKSEETITPWNFEE |
| | VVDKGASAQSFIERMTNFDKNLPNEKVLPKHSLLYEYFTVYN |
| | ELTKVKYVTEGMRKPAFLSGEQKKAIVDLLFKTNRKVTVKQL |
| | KEDYFKKIECFDSVEISGVEDRFNASLGTYHDLLKIIKDKDFLD |
| | NEENEDILEDIVLTLTLFEDREMIEERLKTYAHLFDDKVMKQL |
| | KRRRYTGWGRLSRKLINGIRDKQSGKTILDFLKSDGFANRNFM |
| | QLIHDDSLTFKEDIQKAQVSGQGDSLHEHIANLAGSPAIKKGIL |
| | QTVKVVDELVKVMGRHKPENIVIEMARENQTTQKGQKNSRE |
| | RMKRIEEGIKELGSQILKEHPVENTQLQNEKLYLLQNGRDMY |
| | VDQELDINRLSDYDVDAIVPQSFLKDDSIDNKVLTRSDKNRGK |
| | SDNVPSEEVVKKMKNYWRQLLNAKLITQRKFDNLTKAERGG |
| | LSELDKAGFIKRQLVETRQITKHVAQILDSRMNTKYDENDKLI |
| | REVKVITLKSKLVSDFRKDFQFYKVREINNYHHAHDAYLNAV |
| | VGTALIKKYPKLESEFVYGDYKVYDVRKMIAKSEQEIGKATA |
| | KYFFYSNIMNFFKTEITLANGEIRKRPLIETNGETGEIVWDKGR |
| | DFATVRKVLSMPQVNIVKKTEVQTGGFSKESILPKRNSDKLIA |
| | RKKDWDPKKYGGFDSPTVAYSVLVVAKVEKGKSKKLKSVKE |
| | LLGITIMERSSFEKNPIDFLEAKGYKEVKKDLIIKLPKYSLFELE |
| | NGRKRMLASAGELQKGNELALPSKYVNFLYLASHYEKLKG |

TABLE 4
Sequences of the C-terminus portion of the SpyCas9 H840A Nickase

| | |
|---|---|
| C-terminus | KNGYAGYIDGGASQEEFYKFIKPILEKMDGTEELLVKLNREDL |
| portion of the | LRKQRTFDNGSIPHQIHLGELHAILRRQEDFYPFLKDNREKIEKI |
| Cas9 nickase of | LTFRIPVGPLARGNSRFAWMTRKSEETITPWNFEEVVDKGASA |
| iM-S355-PE | QSFIERMTNFDKNLPNEKVLPKHSLLYEYFTVYNELTKVKYVT |
| (SEQ ID NO: 11) | EGMRKPAFLSGEQKKAIVDLLFKTNRKVTVKQLKEDYFKKIEC |
| | FDSVEISGVEDRFNASLGTYHDLLKIIKDKDFLDNEENEDILEDI |
| | VLTLTLFEDREMIEERLKTYAHLFDDKVMKQLKRRRYTGWGR |
| | LSRKLINGIRDKQSGKTILDFLKSDGFANRNFMQLIHDDSLTFK |
| | EDIQKAQVSGQGDSLHEHIANLAGSPAIKKGILQTVKVVDELV |
| | KVMGRHKPENIVIEMARENQTTQKGQKNSRERMKRIEEGIKEL |
| | GSQILKEHPVENTQLQNEKLYLLQNGRDMYVDQELDINRLSD |
| | YDVDAIVPQSFLKDDSIDNKVLTRSDKNRGKSDNVPSEEVVKK |
| | MKNYWRQLLNAKLITQRKFDNLTKAERGGLSELDKAGFIKRQ |
| | LVETRQITKHVAQILDSRMNTKYDENDKLIREVKVITLKSKLV |
| | SDFRKDFQFYKVREINNYHHAHDAYLNAVVGTALIKKYPKLE |
| | SEFVYGDYKVYDVRKMIAKSEQEIGKATAKYFFYSNIMNFFKT |
| | EITLANGEIRKRPLIETNGETGEIVWDKGRDFATVRKVLSMPQ |
| | VNIVKKTEVQTGGFSKESILPKRNSDKLIARKKDWDPKKYGGF |
| | DSPTVAYSVLVVAKVEKGKSKKLKSVKELLGITIMERSSFEKN |
| | PIDFLEAKGYKEVKKDLIIKLPKYSLFELENGRKRMLASAGELQ |
| | KGNELALPSKYVNFLYLASHYEKLKGSPEDNEQKQLFVEQHK |
| | HYLDEIIEQISEFSKRVILADANLDKVLSAYNKHRDKPIREQAE |
| | NIIHLFTLTNLGAPAAFKYFDTTIDRKRYTSTKEVLDATLIHQSI |
| | TGLYETRIDLSQLGGD |
| C-terminus | QEIGKATAKYFFYSNIMNFFKTEITLANGEIRKRPLIETNGETGE |
| portion of the | IVWDKGRDFATVRKVLSMPQVNIVKKTEVQTGGFSKESILPKR |
| Cas9 nickase of | NSDKLIARKKDWDPKKYGGFDSPTVAYSVLVVAKVEKGKSK |
| IMM-E1026-PE | KLKSVKELLGITIMERSSFEKNPIDFLEAKGYKEVKKDLIIKLPK |
| (SEQ ID NO: 12) | YSLFELENGRKRMLASAGELQKGNELALPSKYVNFLYLASHY |
| | EKLKGSPEDNEQKQLFVEQHKHYLDEIIEQISEFSKRVILADAN |
| | LDKVLSAYNKHRDKPIREQAENIIHLFTLTNLGAPAAFKYFDTT |
| | IDRKRYTSTKEVLDATLIHQSITGLYETRIDLSQLGGD |
| C-terminus | GEIRKRPLIETNGETGEIVWDKGRDFATVRKVLSMPQVNIVKK |
| portion of the | TEVQTGGFSKESILPKRNSDKLIARKKDWDPKKYGGFDSPTVA |
| Cas9 nickase of | YSVLVVAKVEKGKSKKLKSVKELLGITIMERSSFEKNPIDFLEA |
| iMM-N1054-PE | KGYKEVKKDLIIKLPKYSLFELENGRKRMLASAGELQKGNELA |
| (SEQ ID NO: 13) | LPSKYVNFLYLASHYEKLKGSPEDNEQKQLFVEQHKHYLDEII |
| | EQISEFSKRVILADANLDKVLSAYNKHRDKPIREQAENIIHLFTL |
| | TNLGAPAAFKYFDTTIDRKRYTSTKEVLDATLIHQSITGLYETR |
| | IDLSQLGGD |
| C-terminus | SPEDNEQKQLFVEQHKHYLDEIIEQISEFSKRVILADANLDKVL |
| portion of the | SAYNKHRDKPIREQAENIIHLFTLTNLGAPAAFKYFDTTIDRKR |
| Cas9 nickase of | YTSTKEVLDATLIHQSITGLYETRIDLSQLGGD |
| iMM-G1247-PE | |
| & nMM-iMM- | |
| G1247-PE | |
| (SEQ ID NO: 14) | |
| C-terminus | KPIREQAENIIHLFTLTNLGAPAAFKYFDTTIDRKRYTSTKEVLD |
| portion of the | ATLIHQSITGLYETRIDLSQLGGD |
| Cas9 nickase of | |
| iMM-D1299-PE | |
| (SEQ ID NO: 15) | |
| C-terminus | LDINRLSDYDVDAIVPQSFLKDDSIDNKVLTRSDKNRGKSDNV |
| portion of the | PSEEVVKKMKNYWRQLLNAKLITQRKFDNLTKAERGGLSELD |
| Cas9 nickase of | KAGFIKRQLVETRQITKHVAQILDSRMNTKYDENDKLIREVKV |
| iMM-E827-PE | ITLKSKLVSDFRKDFQFYKVREINNYHHAHDAYLNAVVGTALI |
| (SEQ ID NO: 16) | KKYPKLESEFVYGDYKVYDVRKMIAKSEQEIGKATAKYFFYS |
| | NIMNFFKTEITLANGEIRKRPLIETNGETGEIVWDKGRDFATVR |
| | KVLSMPQVNIVKKTEVQTGGFSKESILPKRNSDKLIARKKDWD |
| | PKKYGGFDSPTVAYSVLVVAKVEKGKSKKLKSVKELLGITIME |
| | RSSFEKNPIDFLEAKGYKEVKKDLIIKLPKYSLFELENGRKRML |
| | ASAGELQKGNELALPSKYVNFLYLASHYEKLKGSPEDNEQKQ |
| | LFVEQHKHYLDEIIEQISEFSKRVILADANLDKVLSAYNKHRDK |
| | PIREQAENIIHLFTLTNLGAPAAFKYFDTTIDRKRYTSTKEVLDA |
| | TLIHQSITGLYETRIDLSQLGGD |
| C-terminus | GGLSELDKAGFIKRQLVETRQITKHVAQILDSRMNTKYDENDK |
| portion of the | LIREVKVITLKSKLVSDFRKDFQFYKVREINNYHHAHDAYLNA |
| Cas9 nickase of | VVGTALIKKYPKLESEFVYGDYKVYDVRKMIAKSEQEIGKAT |
| iMM-delta(S793- | AKYFFYSNIMNFFKTEITLANGEIRKRPLIETNGETGEIVWDKG |
| R905)-PE | RDFATVRKVLSMPQVNIVKKTEVQTGGFSKESILPKRNSDKLI |
| (SEQ ID NO: 17) | ARKKDWDPKKYGGFDSPTVAYSVLVVAKVEKGKSKKLKSVK |
| | ELLGITIMERSSFEKNPIDFLEAKGYKEVKKDLIIKLPKYSLFEL |
| | ENGRKRMLASAGELQKGNELALPSKYVNFLYLASHYEKLKGS |
| | PEDNEQKQLFVEQHKHYLDEIIEQISEFSKRVILADANLDKVLS |
| | AYNKHRDKPIREQAENIIHLFTLTNLGAPAAFKYFDTTIDRKRY |
| | TSTKEVLDATLIHQSITGLYETRIDLSQLGGD |
| C-terminus | SPEDNEQKQLFVEQHKHYLDEIIEQISEFSKRVILADANLDKVL |
| portion of the | SAYNKHRDKPIREQAENIIHLFTLTNLGAPAAFKYFDTTIDRKR |
| Cas9 nickase of | YTSTKEVLDATLIHQSITGLYETRIDLSQLGGD |
| nMM-iMM- | |
| G1247-PE | |
| (SEQ ID NO: 18) | |

Prime editors enable deletion, insertion, and base substitution without double-strand breaks. Anzalone et al., “Search-and-replace genome editing without double-strand breaks or donor DNA” Nature 576:149-157 (2019). However, this known fusion of a Cas9 nickase (nCas9; PE2) and a Moloney murine leukemia virus nucleotide polymerase (M-MLV RT) is >6.3 kb. This size is beyond the packaging capacity of a single adeno-associated virus (AAV).

Production of such a large protein in recombinant form in high yield to accommodate ribonucleoprotein (RNP) delivery can also be challenging. Some split Cas9 fusion construct strategies have been tested for the delivery of genome editing tools, including split inteins and MS2 or SunTag tethers. However, most of those split Cas9 fusion construct approaches have not yet been applied to prime editors. Wang et al., “CRISPR-Based Therapeutic Genome Editing: Strategies and In Vivo Delivery by AAV Vectors” Cell 181:136-150 (2020): Truong et al., “Development of an intein-mediated split-Cas9 system for gene therapy” Nucleic Acids Res 43:6450-6458 (2015); Maji et al., “Multidimensional chemical control of CRISPR-Cas9” Nat Chem Biol 13:9-11 (2017)” Liu et al., “A chemical-inducible CRISPR-Cas9 system for rapid control of genome editing” Nat Chem Biol 12:980-987 (2016): Li et al., “SWISS: multiplexed orthogonal genome editing in plants with a Cas9 nickase and engineered CRISPR RNA scaffolds” Genome Biol 21:141 (2020): Konermann et al., “Genome-scale transcriptional activation by an engineered CRISPR-Cas9 complex” Nature 517:583-588 (2015)” Wang et al., “sgBE: a structure-guided design of sgRNA architecture specifies base editing window and enables simultaneous conversion of cytosine and adenosine” Genome Biol 21:222 (2020); Jiang et al., “BE-PLUS: a new base editing tool with broadened editing window and enhanced fidelity” Cell Res 28:855-861 (2018).

These previously reported PE systems may also include a conjugated RNA that consists of a single guide RNA (sgRNA), a 3′ extension containing the NP template NPT nucleotide and a primer binding site (PBS), referred to herein as a prime editor sgRNA (e.g., pegRNA). Despite their usefulness, such pegRNAs are prone to misfolding due to inevitable inappropriate base pairing between the PBS and a spacer, as well as potential NPT-scaffold binding interactions. Finally, the 3′-terminal extension in the pegRNA is exposed to the cytosol and is therefore susceptible to degradation by nucleases, which may compromise the integrity of the pegRNA. Therefore, efforts to reduce pegRNA misfolding and instability are needed.

### II. Conventional Split And Modular Prime Editor Constructs

Previously reported split prime editor fusion constructs include, but are not limited to, an MS2-PE2 and SunTag-PE2 fusion constructs. MS2-PE2 comprises an MS2 coat protein (MCP) fused to the N-terminus of an M-MLV RT protein. Multiple cognate MS2-pegRNAs were engineered by incorporating MS2 stem-loops into different positions of the sgRNA. Additionally, a split SunTag fusion construct was created by fusing an scFv protein fragment to an N-terminus of M-MLV RT protein. Subsequently, the SunTag scFv-RT fusion construct was recruited by either GCN4-nCas9 or nCas9-GCN4. These two PE2 fusion constructs are generally referred to as SunTag-PE2 (GCN4-nCas9) and PE2-SunTag (nCas9-GCN4) based on domain order of elements.

The MS2, SunTag and sPE platforms have been designated in the art as a prime editor (PE3) format. The PE3 format differs from PE2 by inclusion of an additional sgRNA that directs nicking of the unedited strand, thereby biasing repair. The respective nCas9-, RT-, pegRNA-, and nicking sgRNA-expressing plasmids were co-transfected into a HEK293T-derived mCherry reporter lentivector-transduced cell line with a premature TAG stop codon that can be reverted to wild type codon, yielding a red fluorescence signal. Liu et al., “Improved prime editors enable pathogenic allele correction and cancer modelling in adult mice” Nat Commun 12:21 (2021). The most potent MS2- and SunTag-tethered configurations were comparable in editing efficiency to a PE3 fusion construct.

### III. Split Prime Editor Constructs

Attempts have been made to fuse 2 MCP proteins to cas9 to recruit MCP containing petRNA. However, the editing efficiencies of the previously reported split prime editing constructs are not yet fully optimized. One possible reason for modest or inconsistent activity is the use of split Cas9-H840A and MCP-RT. Herein, optimized new modular prime editor constructs were engineered to improve petRNA-based and L-pet-based prime editing. These novel modular prime editors were designed with a single fused effector instead of a split Cas9 nickase and NP for a more efficient recruitment of petRNA. Moreover, the MCP protein has been suggested to be an obligate homodimer. Therefore, the use of MCP dimers or multimers instead of an MCP monomer may improve binding and recruitment of petRNA.

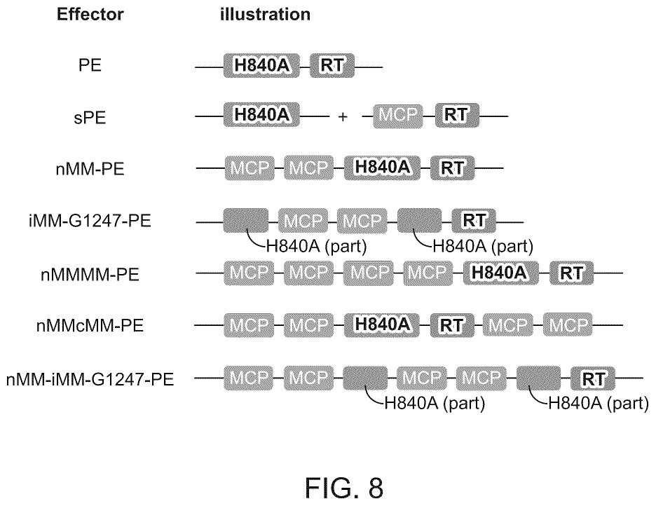

In one aspect, the disclosure provides a modular prime editing system, comprising: i) a fusion protein comprising a Cas9 nickase protein linked to a nucleotide polymerase (NP) protein; ii) a petRNA comprising a primer binding site, a nucleotide polymerase template (NPT), and at least one MS2 hairpin; and iii) a single guide RNA (sgRNA) wherein the fusion protein comprises at least four MS2 binding proteins (FIG. 8, effectors nMMMM-PE and nMMcMM-PE).

In certain embodiments, the fusion protein consists of four adjacent MS2 binding proteins on the N-terminus (FIG. 8, effector nMMMM-PE). In other embodiments, the fusion protein consists of four adjacent MS2 binding proteins on the C-terminus. In certain embodiments, the fusion protein consists of two adjacent MS2 binding proteins on the N-terminus, and two adjacent MS2 binding proteins on the C-terminus.

In certain embodiments, the fusion protein consists of four nonadjacent MS2 binding proteins on the N-terminus. In other embodiments, the fusion protein consists of four nonadjacent MS2 binding proteins on the C-terminus. In certain embodiments, the fusion protein consists of two nonadjacent MS2 binding proteins on the N-terminus, and two nonadjacent MS2 binding proteins on the C-terminus.

In certain embodiments, the modular prime editing system comprises a fusion protein comprising, from the N-terminus to the C-terminus: four adjacent MS2 binding proteins, the Cas9 nickase protein, and an NP protein (FIG. 8, effector nMMMM-PE); or a first MS2 binding protein, a second MS2 binding protein, the Cas9 nickase protein, an NP protein, a third MS2 binding protein and a fourth MS2 binding protein (FIG. 8, effector nMMcMM-PE); or a first MS2 binding protein, a second MS2 binding protein, the N-terminus portion of the Cas9 nickase protein, a third MS2 binding protein and a fourth MS2 binding protein, the C-terminus portion of the Cas9 nickase protein, and an NP protein (FIG. 8, effector nMM-iMM-G124-PE); or the Cas9 nickase protein, an NP protein, and four adjacent MS2 binding proteins.

In certain embodiments, the modular prime editing system comprises a Cas9 nickase comprising one or more amino acid substitution. In other embodiments, the one or more amino acid substitution in the Cas9 nickase is an H840A substitution.

In certain embodiments, the modular prime editing system comprises: the first MS2 binding protein comprising the sequence of SEQ ID NO: 21; the second MS2 binding protein comprising the sequence of SEQ ID NO: 21; the third MS2 binding protein comprising the sequence of SEQ ID NO: 21; the fourth MS2 binding protein comprising the sequence of SEQ ID NO: 21; the Cas9 nickase protein comprise the sequence of SEQ ID NO: 1, and the NP comprising the sequence of SEQ ID NO: 19.

In one aspect, the disclosure provides a modular prime editing system, comprising: i) a fusion protein comprising a Cas9 nickase protein linked to a nucleotide polymerase (NP) protein; ii) a petRNA comprising a primer binding site, a nucleotide polymerase template (NPT), and at least one MS2 hairpin; and iii) a single guide RNA (sgRNA), wherein the fusion protein comprises at least one MS2 binding protein at the N terminus and at least one MS2 binding protein at the C terminus.

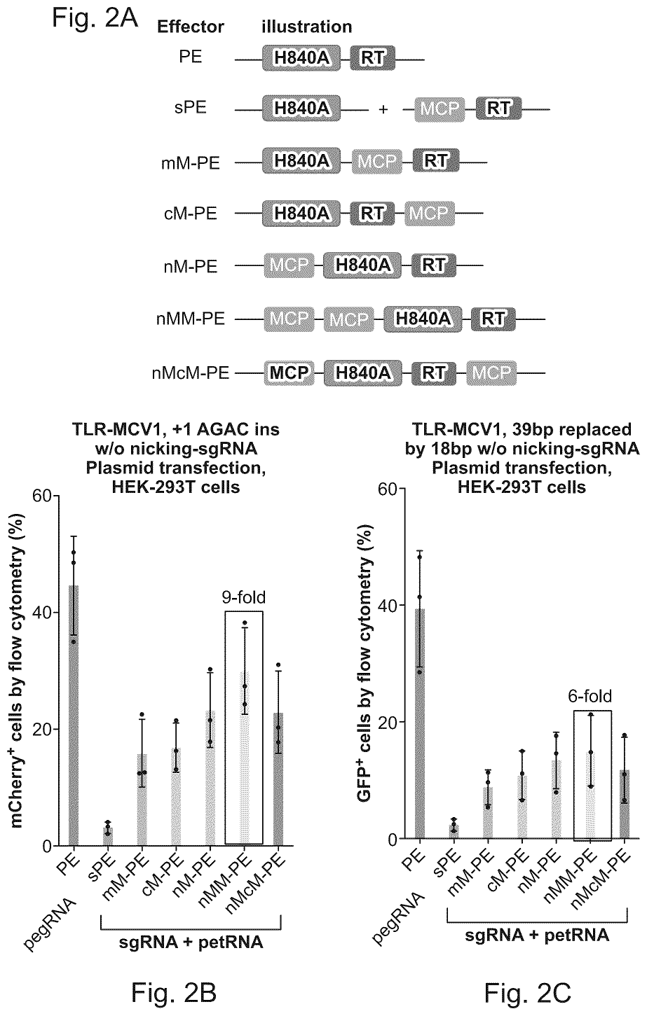

In certain embodiments, the fusion protein consists of one MS2 binding protein on the N-terminus, and one MS2 binding protein on the C-terminus (FIGS. 2A-2C, effector nMcM). In other embodiments, the fusion protein comprises from the N-terminus to the C-terminus: a first MS2 binding protein, the Cas9 nickase protein, an NP protein, and a second MS2 binding protein.

In certain embodiments, the modular prime editing system comprises: the first MS2 binding protein comprising the sequence of SEQ ID NO: 21; the Cas9 nickase protein comprising the sequence of SEQ ID NO: 1, the NP comprising the sequence of SEQ ID NO: 19; and the second MS2 binding protein comprising the sequence of SEQ ID NO: 21.

Previously reported PE systems may include a conjugated RNA that consists of a single guide RNA (sgRNA), a 3′ extension containing the nucleotide polymerase (NP) template (NPT) nucleotide and a primer binding site (PBS), referred to herein as a prime editor sgRNA (e.g., pegRNA). In order to optimize the pegRNA complex for a higher PE efficiency and precision, stem loop aptamer MS2 were appended to the 3′ terminal of pegRNAs (pegRNA-MS2). Feng et al., “Enhancing Prime Editing Efficiency and Flexibility with Tethered and Split pegRNAs” Protein Cell 14 (4): 304-308 (2023). Despite their usefulness, such pegRNAs are prone to misfolding due to inevitable inappropriate base pairing between the PBS and a spacer, as well as potential NPT-scaffold binding interactions. Finally, the 3′-terminal extension in the pegRNA is exposed to the cytosol and is therefore susceptible to degradation by nucleases, which may compromise the integrity of the pegRNA. Therefore, efforts to reduce pegRNA misfolding and instability are needed.

Strategies for the optimization of the prime editing systems provided herein include:

Herein, in one aspect, the disclosure provides a modular prime editing system, comprising: i) a fusion protein comprising a Cas9 nickase protein linked to a nucleotide polymerase (NP) protein; ii) a petRNA comprising a primer binding site, a nucleotide polymerase template (NPT), and at least one MS2 hairpin; and iii) a single guide RNA (sgRNA), wherein the fusion protein comprises at least one MS2 binding protein at the N terminus or at least one MS2 binding protein at the C terminus or at least one MS2 binding protein between the Cas9 nickase and the RT.

In certain embodiments, the fusion protein comprises one MS2 binding protein on the N-terminus (FIG. 2A, effector nM-PE). In other embodiments, the fusion protein comprises one MS2 binding protein on the C-terminus (FIG. 2A, effector cM-PE). In other embodiments, the fusion protein comprises one MS2 binding protein between the Cas9 nickase and the NP (FIG. 2A, effector mM-PE). In other embodiments, the fusion protein comprises two MS2 binding proteins on the N-terminus (FIG. 2A, effector nMM-PE).

In certain embodiments, the modular prime editing system comprises: the MS2 binding protein comprising the sequence of SEQ ID NO: 21, the Cas9 nickase protein comprising the sequence of SEQ ID NO: 1, and the NP comprising the sequence of SEQ ID NO: 19. In certain embodiments, the modular prime editing system comprises: the first MS2 binding protein comprising the sequence of SEQ ID NO: 21, the first MS2 binding protein comprising the sequence of SEQ ID NO: 21, the Cas9 nickase protein comprising the sequence of SEQ ID NO: 1, and the NP comprising the sequence of SEQ ID NO: 19. In certain embodiments, the modular prime editing system comprises: the Cas9 nickase protein comprising the sequence of SEQ ID NO: 1, the MS2 binding protein comprising the sequence of SEQ ID NO: 21, and the NP comprising the sequence of SEQ ID NO: 19. In certain embodiments, the modular prime editing system comprises: the Cas9 nickase protein comprising the sequence of SEQ ID NO: 1, the NP comprising the sequence of SEQ ID NO: 19, and the MS2 binding protein comprising the sequence of SEQ ID NO: 21.

In some embodiments, the nucleotide polymerase of the prime modular editing system is selected from the group consisting of deoxyribonucleic acid polymerase protein (DNAPol), ribonucleic acid polymerase protein (RNAPol), a deoxyribonucleic acid nucleotide polymerase template (dNPT), a ribonucleic acid nucleotide polymerase template (rNPT), and a reverse transcriptase RT. In other embodiments, the nucleotide polymerase of the modular prime editing system is an RT. In certain embodiments, the RT is a Moloney murine leukemia virus RT (M-MLV RT).

TABLE 5
Sequence of the Moloney murine leukemia
virus reverse transcriptase (M-MLV RT)

| | |
|---|---|
| M-MLV RT | TLNIEDEYRLHETSKEPDVSLGSTWLSDFPQ |
| (SEQ ID | AWAETGGMGLAVRQAPLIIPLKATSTPVSI |
| NO: 19) | KQYPMSQEARLGIKPHIQRLLDQGILVPCQ |
| | SPWNTPLLPVKKPGTNDYRPVQDLREVNKR |
| | VEDIHPTVPNPYNLLSGLPPSHQWYTVLDL |
| | KDAFFCLRLHPTSQPLFAFEWRDPEMGISG |
| | QLTWTRLPQGFKNSPTLFNEALHRDLADFR |
| | IQHPDLILLQYVDDLLLAATSELDCQQGTR |
| | ALLQTLGNLGYRASAKKAQICQKQVKYLGY |
| | LLKEGQRWLTEARKETVMGQPTPKTPRQLR |
| | EFLGKAGFCRLFIPGFAEMAAPLYPLTKPG |
| | TLFNWGPDQQKAYQEIKQALLTAPALGLPD |
| | LTKPFELFVDEKQGYAKGVLTQKLGPWRRP |
| | VAYLSKKLDPVAAGWPPCLRMVAAIAVLTK |
| | DAGKLTMGQPLVILAPHAVEALVKQPPDRW |
| | LSNARMTHYQALLLDTDRVQFGPVVALNPA |
| | TLLPLPEEGLQHNCLDILAEAHGTRPDLTD |
| | QPLPDADHTWYTDGSSLLQEGQRKAGAAVT |
| | TETEVIWAKALPAGTSAQRAELIALTQALK |
| | MAEGKKLNVYTDSRYAFATAHIHGEIYRRR |
| | GWLTSEGKEIKNKDEILALLKALFLPKRLS |
| | IIHCPGHQKGHSAEARGNRMADQAARKAAI |
| | TETPDTSTLLIENSSP |

A. Prime Editors with MCPs Inlaid with the Cas9 Nickase.

Exploration of modular prime editing systems with one or more MCP coat proteins (Table 5) inserted at several inlaid positions within the Cas9 nickase was also explored to test its effect on the prime editing efficiencies of the effectors.

TABLE 6
Sequences of the MS2 coat protein
(MCP monomer)

| | |
|---|---|
| MCP monomer | MASNFTQFVLVDNGGTGDVTVAPSNFANGV |
| (if | AEWISSNSRSQAYKVTCSVRQSSAQKRKYT |
| located at | IKVEVPKVATQTVGGVELPVAAWRSYLNME |
| the 5′ end of | LTIPIFATNSDCELIVKAMQGLLKDGNPIP |
| the fusion | SAIAANSGIY |
| protein) | |
| (SEQ ID NO: 20) | |
| MCP monomer | ASNFTQFVLVDNGGTGDVTVAPSNFANGVA |
| (if not | EWISSNSRSQAYKVTCSVRQSSAQKRKYTI |
| located at | KVEVPKVATQTVGGVELPVAAWRSYLNMEL |
| the 5′ end of | TIPIFATNSDCELIVKAMQGLLKDGNPIPS |
| the fusion | AIAANSGIY |
| protein) | |
| (SEQ ID NO: 21) | |

In one aspect, the disclosure provides a modular prime editing system, comprising: i) a fusion protein comprising a Cas9 nickase protein linked to a nucleotide polymerase (NP) protein; ii) a petRNA comprising a primer binding site (PBS), a nucleotide polymerase template (NPT), and at least one MS2 hairpin, and iii) a single guide RNA (sgRNA), wherein the fusion protein comprises at least one MS2 binding protein inlaid within the Cas9 nickase.

In certain embodiments, the fusion protein comprises two or more MS2 binding proteins inlaid within the Cas9 nickase. In certain embodiments, the fusion protein comprises two MS2 binding proteins on the N-terminus, and two MS2 binding proteins inlaid in the Cas9 nickase. In certain embodiments, the fusion protein comprises two MS2 binding proteins on the C-terminus, and two MS2 binding proteins inlaid in the Cas9 nickase.

In certain embodiments, the at least one MS2 binding protein is inlaid within one of the Rec-1, RuvC-III, PID, or HNH domains of the Cas9 nickase. In certain embodiments, the at least one MS2 binding protein is inlaid within the PID domain of the Cas9 nickase. In certain embodiments, the at least one MS2 binding protein is inlaid within position G1247 of the PID domain of the Cas9 nickase of SEQ ID NO: 1. In certain embodiments, the fusion protein comprises two adjacent MS2 binding proteins inlaid within position G1247 of the PID domain of the Cas9 nickase of SEQ ID NO: 1.

In certain embodiments, the modular prime editing system comprises the fusion protein comprising from the N-terminus to the C-terminus: the N-terminus portion of the Cas9 nickase protein, one MS2 binding protein, the C-terminus portion of the Cas9 nickase protein, and an NP protein; or the N-terminus portion of the Cas9 nickase protein, two MS2 binding proteins, the C-terminus portion of the Cas9 nickase protein, and an NP protein.

In other embodiments, the modular prime editing system comprises the fusion protein comprising from the N-terminus to the C-terminus: four adjacent MS2 binding proteins, the Cas9 nickase protein, and an NP protein; or a first MS2 binding protein, a second MS2 binding protein, the Cas9 nickase protein, an NP protein, a third MS2 binding protein and a fourth MS2 binding protein; or a first MS2 binding protein, a second MS2 binding protein, the N-terminus portion of the Cas9 nickase protein, a third MS2 binding protein and a fourth MS2 binding protein, the C-terminus portion of the Cas9 nickase protein, and an NP protein; or the Cas9 nickase protein, an NP protein, and four adjacent MS2 binding proteins.

In certain embodiments, the modular prime editing system comprises: the N-terminus portion of the Cas9 nickase protein comprising the sequence of SEQ ID NO: 2, the MS2 binding protein comprising the sequence of SEQ ID NO: 21, the C-terminus portion of the Cas9 nickase protein comprising the sequence of SEQ ID NO: 11, and the NP protein comprising the sequence of SEQ ID NO: 19 (Effector iM-S355-PE).

In certain embodiments, the modular prime editing system comprises: the N-terminus portion of the Cas9 nickase protein comprising the sequence of SEQ ID NO: 3, the first MS2 binding protein comprising the sequence of SEQ ID NO: 21, the second MS2 binding protein comprising the sequence of SEQ ID NO: 21, the C-terminus portion of the Cas9 nickase comprises the sequence of SEQ ID NO: 12, and the NP protein comprising the sequence of SEQ ID NO: 19 (Effector iMM-E1026-PE).

In certain embodiments, the modular prime editing system comprises: the N-terminus portion of the Cas9 nickase protein comprising the sequence of SEQ ID NO: 4, the first MS2 binding protein comprising the sequence of SEQ ID NO: 21, the second MS2 binding protein comprises the sequence of SEQ ID NO: 21, the C-terminus portion of the Cas9 nickase comprises the sequence of SEQ ID NO: 13, and the NP protein comprising the sequence of SEQ ID NO: 19 (Effector iMM-N1054-PE).

In certain embodiments, the modular prime editing system comprises: the N-terminus portion of the Cas9 nickase protein comprising the sequence of SEQ ID NO: 5, the first MS2 binding protein comprising the sequence of SEQ ID NO: 21, the second MS2 binding protein comprises the sequence of SEQ ID NO: 21, the C-terminus portion of the Cas9 nickase comprises the sequence of SEQ ID NO: 14, and the NP protein comprising the sequence of SEQ ID NO: 19 (Effector iMM-G1247-PE)

In certain embodiments, the modular prime editing system comprises: the N-terminus portion of the Cas9 nickase protein comprising the sequence of SEQ ID NO: 6, the first MS2 binding protein comprising the sequence of SEQ ID NO: 21, the second MS2 binding protein comprises the sequence of SEQ ID NO: 21, the C-terminus portion of the Cas9 nickase comprises the sequence of SEQ ID NO: 15, and the NP protein comprising the sequence of SEQ ID NO: 19 (effector iMM-D1299-PE).

In certain embodiments, the modular prime editing system comprises: the N-terminus portion of the Cas9 nickase protein comprising the sequence of SEQ ID NO: 7, the first MS2 binding protein comprising the sequence of SEQ ID NO: 21, the second MS2 binding protein comprises the sequence of SEQ ID NO: 21, the C-terminus portion of the Cas9 nickase comprises the sequence of SEQ ID NO: 16, and the NP protein comprising the sequence of SEQ ID NO: (Effector iMM-E827-PE).

In certain embodiments, the modular prime editing system comprises: the N-terminus portion of the Cas9 nickase protein comprising the sequence of SEQ ID NO: 8, the first MS2 binding protein comprising the sequence of SEQ ID NO: 21, the second MS2 binding protein comprises the sequence of SEQ ID NO: 21, the C-terminus portion of the Cas9 nickase comprises the sequence of SEQ ID NO: 17, and the NP protein comprising the sequence of SEQ ID NO: 19 (Effector iMM-delta (S793-R905)-PE).

In certain embodiments, the modular prime editing system comprises: the first MS2 binding protein comprises the sequence of SEQ ID NO: 21, the second MS2 binding protein comprises the sequence of SEQ ID NO: 21, the third MS2 binding protein comprises the sequence of SEQ ID NO: 21, the fourth MS2 binding protein comprises the sequence of SEQ ID NO: 21, the Cas9 nickase protein comprise the sequence of SEQ ID NO: 1, and the NP comprises the sequence of SEQ ID NO: 19.

In certain embodiments, the modular prime editing system comprises: the first MS2 binding protein comprises the sequence of SEQ ID NO: 21, the second MS2 binding protein comprises the sequence of SEQ ID NO: 21, the Cas9 nickase protein comprise the sequence of SEQ ID NO: 1, the NP comprises the sequence of SEQ ID NO: 19, the third MS2 binding protein comprises the sequence of SEQ ID NO: 21, and the fourth MS2 binding protein comprises the sequence of SEQ ID NO: 21.

In certain embodiments, the modular prime editing system comprises: the first MS2 binding protein comprises the sequence of SEQ ID NO: 21, the second MS2 binding protein comprises the sequence of SEQ ID NO: 21, the N-terminus portion of the Cas9 nickase protein comprises the sequence of SEQ ID NO: 9, the third MS2 binding protein comprises the sequence of SEQ ID NO: 21, the fourth MS2 binding protein comprises the sequence of SEQ ID NO: 21, the N-terminus portion of the Cas9 nickase protein comprises the sequence of SEQ ID NO: 18, and the NP comprises the sequence of SEQ ID NO: 19.

### B. Split PetRNA

The prime editor template RNA or petRNA molecule as used herein, refers to an RNA molecule that encodes a primer binding site (PBS) and a nucleotide polymerase template (NPT), that is unattached to the single guide RNA (sgRNA). The petRNA may also encode stem loops. The petRNA may also be linear or circularized. Modifications to the petRNA can enable the prime editing potential of modular prime editing systems. The chemically modified petRNA molecules of the disclosure possess improved in vivo stability, improved genome editing efficacy, and/or reduced immunotoxicity relative to unmodified or minimally modified guide RNAs.

In certain aspects, petRNA a comprises a primer binding site, a nucleotide polymerase template (NPT), at least one MS2 hairpin, and at least one chemically modified nucleotide. In certain embodiments, the one or more modified nucleotides comprise a modification of a ribose group, a phosphate group, a nucleobase, or a combination thereof. In certain embodiments, the modification of the ribose group is selected from 2′-O-methyl, 2′-fluoro, 2′-deoxy, 2′-O-(2-methoxyethyl) (MOE), or 2′-NH2. In certain embodiments, the modification of the phosphate group comprises a phosphorothioate, phosphonoacetate (PACE), thiophosphonoacetate (thioPACE), amide, triazole, phosphonate, or phosphotriester modification.

In certain embodiments, the modified phosphate group comprises at least one phosphorothioate internucleotide linkage. In certain embodiments, the modified phosphate group comprises between 1 and 30 phosphorothioate internucleotide linkages (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 phosphorothioate internucleotide linkages).

In certain embodiments, the modified phosphate group comprises at least one phosphorothioate internucleotide linkage on the primer binding site (PBS). In certain embodiments, the modified phosphate group comprises exactly two phosphorothioate internucleotide linkages on the PBS. In certain embodiments, the modified phosphate group comprises exactly three phosphorothioate internucleotide linkages on the PBS. In certain embodiments, the modified phosphate group comprises between 1 and 10 phosphorothioate internucleotide linkage on the PBS (i.e., 1, 2, 3, 4, 5, 6, 7, 8, 9, or 10 phosphorothioate internucleotide linkages on the PBS).

In certain embodiments, the modification of the nucleobase group is selected from 2-thiouridine, 4-thiouridine, N⁶-methyladenosine, pseudouridine, 2, 6-diaminopurine, inosine, thymidine, 5-methylcytosine, 5-substituted pyrimidine, isoguanine, isocytosine, or halogenated aromatic groups.

In certain embodiments, said petRNA comprises one MS2 hairpin. In other embodiments, the petRNA comprises two MS2 hairpins. In other embodiments, the petRNA comprises three MS2 hairpins. In other embodiments, the petRNA comprises four MS2 hairpins.

In certain embodiments, the at least one MS2 hairpin is chemically modified. In certain embodiments, the one or more modified nucleotides of the MS2 hairpin comprises a modification of a ribose group, a phosphate group, a nucleobase, or a combination thereof. In certain embodiments, the modified MS2 hairpin comprises a phosphate group comprising a phosphorothioate, phosphonoacetate (PACE), thiophosphonoacetate (thioPACE), amide, triazole, phosphonate, or phosphotriester modification.

In certain embodiments, the modified MS2 hairpin comprises a phosphate group comprising at least one phosphorothioate internucleotide linkage. In certain embodiments, the phosphate group comprises three, ten, or twenty-three phosphorothioate internucleotide linkages. In certain embodiments, the phosphate group comprises between 1 and 30 phosphorothioate internucleotide linkages (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 phosphorothioate internucleotide linkages).

In certain embodiments, the phosphorothioate internucleotide linkages are located on the N terminus. In other embodiments, the phosphorothioate internucleotide linkages are located on the C terminus.

In certain embodiments, the modification of the nucleobase group is selected from 2-thiouridine, 4-thiouridine, N⁶-methyladenosine, pseudouridine, 2, 6-diaminopurine, inosine, thymidine, 5-methylcytosine, 5-substituted pyrimidine, isoguanine, isocytosine, or halogenated aromatic groups.

In certain embodiments, the petRNA comprises a fully modified MS2 hairpin (i.e. 100% chemically modified MS2 hairpin).

### C. Linkers

Linkers were used to ligate components of the module prime editing system to each other. These include amino acid linkers to fuse the one or more MS2 coat proteins to each other and/or to other components of the modular prime editing system.

Exemplary linkers include, but are not limited to, an ethylene glycol chain, an alkyl chain, a polypeptide, a polysaccharide, a block copolymer, and the like (Table 7).

TABLE 7
Linker Sequences

| | |
|---|---|
| Linker 1 | SGGSSGGSSGSETPGTSESATPES |
| (SEQ ID NO: 26) | SGGSSGGSS |
| Linker 2 | SGGS |
| (SEQ ID NO: 27) | |
| Linker 3 | MA |
| (SEQ ID NO: 28) | |
| Linker 4 | GIHGVPAA |
| (SEQ ID NO: 29) | |
| Linker 5 | SAGGGGSGGGGSGGGGSGPKKKRK |
| (SEQ ID NO: 30) | VAAAGS |
| Linker 6 | GSSGSETPGTSESATPESSG |
| (SEQ ID NO: 31) | |
| Linker 7 | GGSGGSGGSGGSGGSGGSGG |
| (SEQ ID NO: 32) | |
| Linker 8 | SAGGGGSGGGGSGGGGSG |
| (SEQ ID NO: 33) | |
| Linker 9 | SGGSSGGSSGGSSGGS |
| (SEQ ID NO: 34) | |

In certain embodiments, the fusion protein comprised at least one MS2 binding protein inlaid within the Cas9 nickase, wherein the one or more MS2 binding proteins are attached to the Cas9 nickase via one or more linkers.

In certain embodiments, the one or more MS2 binding proteins are attached to the Cas9 nickase via two linkers, wherein a first linker is on the N-terminus of the Cas9 nickase, and a second linker is on the C-terminus of the Cas9 nickase.

In certain embodiments, the one or more MS2 binding proteins are attached to each other via one or more linker. In other embodiments, the one or more MS2 binding proteins are attached to each other via one linker and to the Cas9 nickase via two linkers.

In certain embodiments, the one or more MS2 binding proteins are attached to each other via one linker and to the Cas9 nickase via two linkers, wherein the first linker is on the N-terminus of the Cas9 nickase, and the second linker is on the C-terminus of the Cas9 nickase.

In certain embodiments, the two MS2 binding proteins inlaid within the Cas9 nickase are attached to each other via one linker and to the Cas9 nickase via two linkers, wherein the first linker is on the N-terminus of the Cas9 nickase, and the second linker is on the C-terminus of the Cas9 nickase.

In certain embodiments, the fusion protein comprises from the N-terminus to the C-terminus: the N-terminus portion of the Cas9 nickase protein, a first linker, one MS2 binding protein, a second linker, the C-terminus portion of the Cas9 nickase protein, a third linker, and an NP protein; or the N-terminus portion of the Cas9 nickase protein, a first linker, a first MS2 binding protein, a second linker, a second MS2 binding protein, a third linker, the C-terminus portion of the Cas9 nickase protein, a fourth linker, and an NP protein.

In certain embodiments, the modular prime editing system comprises: the N-terminus portion of the Cas9 nickase protein comprising the sequence of SEQ ID NO: 2, the first linker comprising the sequence of SEQ ID NO: 31, the MS2 binding protein comprising the sequence of SEQ ID NO: 21, the second linker comprising the sequence of SEQ ID NO: 32, the C-terminus portion of the Cas9 nickase protein comprising the sequence of SEQ ID NO: 11, the third linker comprising the sequence of SEQ ID NO: 26, and the NP protein comprising the sequence of SEQ ID NO: 19 (iM-S355-PE).

In certain embodiments, the modular prime editing system comprises: the N-terminus portion of the Cas9 nickase protein comprising the sequence of SEQ ID NO: 3, the first linker comprises the sequence of SEQ ID NO: 34, the first MS2 binding protein comprising the sequence of SEQ ID NO: 21, the second linker comprises the sequence of SEQ ID NO: 31, the second MS2 binding protein comprising the sequence of SEQ ID NO: 21, the third linker comprises the sequence of SEQ ID NO: 33; the C-terminus portion of the Cas9 nickase comprises the sequence of SEQ ID NO: 12, the fourth linker comprises the sequence of SEQ ID NO: 33, and the NP protein comprising the sequence of SEQ ID NO: 19 (iMM-E1026-PE).

In certain embodiments, the modular prime editing system comprises: the N-terminus portion of the Cas9 nickase protein comprising the sequence of SEQ ID NO: 4, the first linker comprises the sequence of SEQ ID NO: 34, the first MS2 binding protein comprising the sequence of SEQ ID NO: 21, the second linker comprises the sequence of SEQ ID NO: 31, the second MS2 binding protein comprising the sequence of SEQ ID NO: 21, the third linker comprises the sequence of SEQ ID NO: 33, the C-terminus portion of the Cas9 nickase comprises the sequence of SEQ ID NO: 13, the fourth linker comprises the sequence of SEQ ID NO: 26, and the NP protein comprising the sequence of SEQ ID NO: 19 (iMM-N1054-PE).

In certain embodiments, the modular prime editing system comprises: the N-terminus portion of the Cas9 nickase protein comprising the sequence of SEQ ID NO: 5, the first linker comprises the sequence of SEQ ID NO: 34, the first MS2 binding protein comprising the sequence of SEQ ID NO: 21, the second linker comprises the sequence of SEQ ID NO: 31, the second MS2 binding protein comprising the sequence of SEQ ID NO: 21, the C-terminus portion of the Cas9 nickase comprises the sequence of SEQ ID NO: 14, the third linker comprises the sequence of SEQ ID NO: 33, and the NP protein comprising the sequence of SEQ ID NO: 19 (iMM-G1247-PE).

In certain embodiments, the modular prime editing system comprises: the N-terminus portion of the Cas9 nickase protein comprising the sequence of SEQ ID NO: 6, the first linker comprises the sequence of SEQ ID NO: 34, the first MS2 binding protein comprising the sequence of SEQ ID NO: 21, the second linker comprises the sequence of SEQ ID NO: 31, the second MS2 binding protein comprising the sequence of SEQ ID NO: 21, the third linker comprises the sequence of SEQ ID NO: 33, the C-terminus portion of the Cas9 nickase comprises the sequence of SEQ ID NO: 15, the fourth linker comprises the sequence of SEQ ID NO: 26, and the NP protein comprising the sequence of SEQ ID NO: 19 (iMM-D1299-PE)

In certain embodiments, the modular prime editing system comprises: the N-terminus portion of the Cas9 nickase protein comprising the sequence of SEQ ID NO: 7, the first linker comprises the sequence of SEQ ID NO: 34, the first MS2 binding protein comprising the sequence of SEQ ID NO: 21, the second linker comprises the sequence of SEQ ID NO: 31, the second MS2 binding protein comprising the sequence of SEQ ID NO: 21, the third linker comprises the sequence of SEQ ID NO: 33, the C-terminus portion of the Cas9 nickase comprises the sequence of SEQ ID NO: 16, the fourth linker comprises the sequence of SEQ ID NO: 26, and the NP protein comprising the sequence of SEQ ID NO: 19 (IMM-E827-PE).

In certain embodiments, the modular prime editing system comprises: the N-terminus portion of the Cas9 nickase protein comprising the sequence of SEQ ID NO: 8, the first linker comprises the sequence of SEQ ID NO: 34, the first MS2 binding protein comprising the sequence of SEQ ID NO: 21, the second linker comprises the sequence of SEQ ID NO: 21, the second MS2 binding protein comprising the sequence of SEQ ID NO: 21, the third linker comprises the sequence of SEQ ID NO: 33, the C-terminus portion of the Cas9 nickase comprises the sequence of SEQ ID NO: 17, the fourth linker comprises the sequence of SEQ ID NO: 26, and the NP protein comprising the sequence of SEQ ID NO: 19 (iMM-delta (S793-R905)-PE).

In certain embodiments, the at least four MS2 binding proteins are attached to the Cas9 nickase via one or more linker. In certain embodiments, the at least four MS2 binding proteins are attached to the Cas9 nickase via two linkers.

In certain embodiments, the at least four MS2 binding proteins are attached to the Cas9 nickase via two linkers, wherein a first linker is on the N-terminus of the Cas9 nickase, and a second linker is on the C-terminus of the Cas9 nickase.

In certain embodiments, the at least four MS2 binding proteins are attached to each other via one or more linker. In certain embodiments, the at least four MS2 binding proteins are attached to each other via one or more linker and to the Cas9 nickase via one or more linker. In certain embodiments, the at least four MS2 binding proteins are attached to each other via one linker and to the Cas9 nickase via two linkers. In certain embodiments, the at least four MS2 binding proteins are attached to each other via one linker and to the Cas9 nickase via two linkers, wherein the first linker is on the N-terminus of the Cas9 nickase, and the second linker is on the C-terminus of the Cas9 nickase.

In certain embodiments, the fusion protein comprises from the N-terminus to the C-terminus: A first MS2 binding protein, a first linker, a second MS2 binding protein, a second linker, a third MS2 protein, a third linker, a fourth MS2 protein, a fourth linker, the Cas9 nickase protein, a fifth linker, and an NP protein; or A first MS2 binding protein, a first linker, a second MS2 binding protein, a second linker, the Cas9 nickase protein, a third linker, an NP protein, a fourth linker, a third MS2 binding protein, a fifth linker, and a fourth MS2 protein; or A first MS2 binding protein, a first linker, a second MS2 binding protein, a second linker, the N-terminus portion of the Cas9 nickase protein, a third linker, a third MS2 binding protein, a fourth linker, a fourth MS2 protein, a fifth linker, the C-terminus portion of the Cas9 nickase protein, and an NP protein, or The Cas9 nickase protein, a first linker, and an NP protein, a second linker, a first MS2 binding protein, a third linker, a second MS2 binding protein, a fourth linker, a third MS2 protein, a fifth linker, and a fourth MS2 protein.

In certain embodiments, the modular prime editing system comprises: the first MS2 binding protein comprising the sequence of SEQ ID NO: 21 the first linker comprising the sequence of SEQ ID NO: 31, the second MS2 binding protein comprising the sequence of SEQ ID NO: 21, the second linker comprising the sequence of SEQ ID NO: 33, the third MS2 binding protein comprising the sequence of SEQ ID NO: 21, the third linker comprising the sequence of SEQ ID NO: 31, the fourth MS2 binding protein comprising the sequence of SEQ ID NO: 21, the fourth linker comprising the sequence of SEQ ID NO: 39, the Cas9 nickase protein comprise the sequence of SEQ ID NO: 1, the fifth linker comprising the sequence of SEQ ID NO: 26, and the NP comprising the sequence of SEQ ID NO: 19 (nMMMM-PE).

In certain embodiments, the modular prime editing system comprises: the first MS2 binding protein comprising the sequence of SEQ ID NO: 21, the first linker comprising the sequence of SEQ ID NO: 31, the second MS2 binding protein comprising the sequence of SEQ ID NO: 21, the second linker comprising the sequence of SEQ ID NO: 30, the Cas9 nickase protein comprise the sequence of SEQ ID NO: 1, the third linker comprising the sequence of SEQ ID NO: 26, the NP comprising the sequence of SEQ ID NO: 19, the fourth linker comprising the sequence of SEQ ID NO: 34, the third MS2 binding protein comprising the sequence of SEQ ID NO: 21, the fifth linker comprising the sequence of SEQ ID NO: 31, and the fourth MS2 binding protein comprising the sequence of SEQ ID NO: 21 (nMMcMM-PE).

In certain embodiments, the modular prime editing system comprises: the first MS2 binding protein comprising the sequence of SEQ ID NO: 21, the first linker comprising the sequence of SEQ ID NO: 31, the second MS2 binding protein comprising the sequence of SEQ ID NO: 21, the second linker comprising the sequence of SEQ ID NO: 30, the N-terminus portion of the Cas9 nickase protein comprising the sequence of SEQ ID NO: 9, the third linker comprising the sequence of SEQ ID NO: 34, the third MS2 binding protein comprising the sequence of SEQ ID NO: 21, the fourth linker comprising the sequence of SEQ ID NO: 31, the fourth MS2 binding protein comprising the sequence of SEQ ID NO: 21, the fifth linker comprising the sequence of SEQ ID NO: 30, the C-terminus portion of the Cas9 nickase protein comprising the sequence of SEQ ID NO: 18, the sixth linker comprising the sequence of SEQ ID NO: 26, and the NP comprising the sequence of SEQ ID NO: 19 (nMM-iMM-G1247-PE).

In certain embodiments, the at least one MS2 binding protein at the N terminus and at least one MS2 binding protein at the C terminus are attached to the Cas9 nickase via one or more linker.

In certain embodiments, the at least one MS2 binding protein at the N terminus and at least one MS2 binding protein at the C terminus are attached to the Cas9 nickase via two linkers.

In certain embodiments, the at least one MS2 binding protein at the N terminus and at least one MS2 binding protein at the C terminus are attached to the Cas9 nickase via two linkers, wherein a first linker is on the N-terminus of the Cas9 nickase, and a second linker is on the C-terminus of the Cas9 nickase.

In certain embodiments, the fusion protein comprises from the N-terminus to the C-terminus: a first MS2 binding protein, a first linker, the Cas9 nickase protein, a second linker, an NP protein, a third linker, and a second MS2 binding protein.

In certain embodiments, the fusion protein comprises the first MS2 binding protein comprising the sequence of SEQ ID NO: 21 the first linker comprising the sequence of SEQ ID NO: 30. the Cas9 nickase protein comprising the sequence of SEQ ID NO: 1, the second linker comprising the sequence of SEQ ID NO: 26, the NP comprising the sequence of SEQ ID NO: 19, the third linker comprising the sequence of SEQ ID NO: 26, and the second MS2 binding protein comprising the sequence of SEQ ID NO: 21 (nMcM-PE).

In certain embodiments, the at least one MS2 binding protein at the N terminus or at least one MS2 binding protein at the C terminus or at least one MS2 binding protein between the Cas9 nickase and the NP are attached to the Cas9 nickase via one or more linker.

In certain embodiments, the at least one MS2 binding protein at the N terminus or at least one MS2 binding protein at the C terminus or at least one MS2 binding protein between the Cas9 nickase and the NP are attached to NP via one or more linker.

In certain embodiments, the at least one MS2 binding protein at the N terminus or at least one MS2 binding protein at the C terminus or at least one MS2 binding protein between the Cas9 nickase and the NP are attached to the Cas9 nickase via a first linker and to the NP via a second linker.

In certain embodiments, the at least one MS2 binding protein at the N terminus or at least one MS2 binding protein at the C terminus or at least one MS2 binding protein between the Cas9 nickase and the NP are attached to the Cas9 nickase via a first linker and to the NP via a second linker, wherein the first linker is on the N-terminus of the Cas9 nickase, and a second linker is on the C-terminus of the RT.

In certain embodiments, the fusion protein comprises from the N-terminus to the C-terminus: The MS2 binding protein, a first linker, the Cas9 nickase protein, a second linker and an NP protein; or The Cas9 nickase protein, a first linker, the NP protein, a second linker, and an MS2 binding protein; or The Cas9 nickase protein, a first linker, an MS2 binding protein, a second linker, and the NP protein.

In certain embodiments, the fusion protein comprises: the MS2 binding proteins comprising the sequence of SEQ ID NO: 21, the first linker comprising the sequence of SEQ ID NO: 30, the Cas9 nickase protein comprising the sequence of SEQ ID NO: 1, the second linker comprising the sequence of SEQ ID NO: 26, and the NP comprising the sequence of SEQ ID NO: 19 (nM-PE).

In certain embodiments, the fusion protein comprises: the Cas9 nickase protein comprising the sequence of SEQ ID NO: 1, the first linker comprising the sequence of SEQ ID NO: 31, the MS2 binding proteins comprising the sequence of SEQ ID NO: 21, the second linker comprising the sequence of SEQ ID NO: 26, and the NP comprising the sequence of SEQ ID NO: 19 (mM-PE).

In certain embodiments, the fusion protein comprises: the first MS2 binding protein comprising the sequence of SEQ ID NO: 21, the first linker comprising the sequence of SEQ ID NO: 31, the second MS2 binding protein comprising the sequence of SEQ ID NO: 21, the second linker comprising the sequence of SEQ ID NO: 30, the Cas9 nickase protein comprising the sequence of SEQ ID NO: 1, the third linker comprising the sequence of SEQ ID NO: 26, and the NP comprising the sequence of SEQ ID NO: 19 (nMM-PE).

In certain embodiments, the fusion protein comprises: the Cas9 nickase protein comprising the sequence of SEQ ID NO: 1, the first linker comprising the sequence of SEQ ID NO: 26, the NP comprising the sequence of SEQ ID NO: 19, the second linker comprising the sequence of SEQ ID NO: 26, and the MS2 binding proteins comprising the sequence of SEQ ID NO: 21 (cM-PE).

### PetRNA Linkers

Linkers were used to the one or more MS2 and NPT-PBS sequences to their effect on editing activities of the petRNA.

In one aspect, the disclosure provides a petRNA a comprising a primer binding site, a nucleotide polymerase template (NPT), and at least one MS2 hairpin, wherein the MS2 is linked to the NPT using a linker.

In an embodiment, the MS2 is linked to the NPT using a linker. In an embodiment, the linker is selected from the group consisting of ethylene glycol and polyethylene glycol (PEG). In an embodiment, the PEG is a hexaethylene glycol (HEX). In an embodiment, the HEX comprises the following structure:

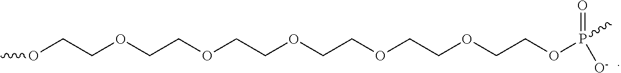

In an embodiment, the PEG is 2×HEX.). In an embodiment, the PEG is 2×HEX comprising the following structure:

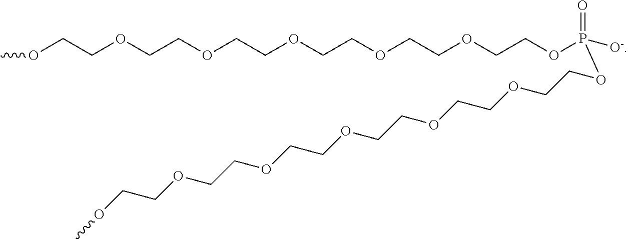

In an embodiment, the linker is a 2′-Omethyl modified RNA. In an embodiment, the 2′-Omethyl modified RNA consists of A and N nucleotide residues. In an embodiment, the 2′-Omethyl modified RNA is between 1 and 15 nucleotides long. In an embodiment, the 2′-Omethyl modified RNA is 5 nucleotides long. In an embodiment, the 2′-Omethyl modified RNA is 10 nucleotides long. In an embodiment, the 2′-Omethyl modified RNA comprises the following sequence from the N-terminus to the C-terminus: AAACACA.

### Adeno-Associated Viruses

Adeno-associated viruses (AAV) are small viruses that infect humans and some other primate species. AAVs are small (20 nm) replication-defective, nonenveloped viruses and have linear single-stranded DNA (ssDNA) genome of approximately 4.8 kilobases (kb). Naso et al. “Adeno-Associated Virus (AAV) as a Vector for Gene Therapy” BioDrugs 31 (4): 317-334 (2017); and Wu et al., “Effect of Genome Size on AAV Vector Packaging” Molecular Therapy 18 (1): 80-86 (2010). AA Vs are not currently known to cause disease. The viruses cause a very mild immune response. Several additional features make AAV an attractive candidate for creating viral vectors for gene therapy, and for the creation of isogenic human disease models. Grieger et al., “Adeno-associated Virus as a Gene Therapy Vector: Vector Development, Production and Clinical Applications”; Adeno-associated virus as a gene therapy vector: vector development, production and clinical applications. In: Advances in Biochemical Engineering/Biotechnology. 99. pp. 119-145 (2005). Gene therapy vectors using AAV can infect both dividing and quiescent cells and persist in an extrachromosomal state without integrating into the genome of the host cell, although in the native virus integration of virally carried genes into the host genome does occur. Deyle et al., “Adeno-associated virus vector integration”. Current Opinion in Molecular Therapeutics. 11 (4): 442-447 (2009).

Development of AAVs as gene therapy vectors eliminated the genomic integration capacity by removal of the rep and cap genes. The modified vector has a promoter to drive transcription of the carried gene which is inserted between inverted terminal repeats (ITRs). AAV-based gene therapy vectors consequently form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Surosky et al., “Adeno-associated virus Rep proteins target DNA sequences to a unique locus in the human genome” Journal of Virology 71 (10): 7951-7959 (1997).

The AAV genome is built of single-stranded deoxyribonucleic acid (ssDNA), cither positive- or negative-sensed, which is about 4.7 kilobase long. The genome comprises ITRs at both ends of the DNA strand, and two open reading frames (ORFs) encoding the rep and cap proteins. The rep ORF is composed of four overlapping genes encoding Rep proteins required for the AAV life cycle. The cap ORF is composed of overlapping nucleotide sequences of capsid proteins (e.g., VP1, VP2 and VP3) which interact to fouli a capsid with icosahedral symmetry. Carter B J, “Aden-associated virus and adeno-associated virus vectors for gene delivery”. In: Lassic D D, Templeton N S (eds.). Gene Therapy: Therapeutic Mechanisms and Strategies. New York City: Marcel Dekker, Inc. pp. 41-59 (2000).

AAV inverted terminal repeat (ITR) sequences usually comprise about 145 bases each and are believed required for efficient multiplication of the AAV genome. Bohenzky et al., “Sequence and symmetry requirements within the internal palindromic sequences of the adeno-associated virus terminal repeat” Virology 166 (2): 316-327 (1988). ITRs also have a hairpin structure which contributes to self-priming that allows a primase-independent synthesis of the second DNA strand. The ITRs were also shown to be required for host cell DNA integration/removal, efficient encapsidation and deoxyribonuclease resistance. Wang et al., “Rescue and replication signals of the adeno-associated virus 2 genome” Journal of Molecular Biology 250 (5): 573-580 (1995); Weitzman et al., “Adeno-associated virus (AAV) Rep proteins mediate complex formation between AAV DNA and its integration site in human DNA” PNAS USA 91 (13): 5808-5812 (1994); and Zhou et al, “In vitro packaging of adeno-associated virus DNA”. Journal of Virology 72 (4): 3241-3247 (1998). With regard to gene therapy, ITRs are configured in cis next to the therapeutic gene, in contrast the structural (cap) and packaging (rep) proteins which can be delivered in trans. Nony et al., “Novel cis-acting replication element in the adeno-associated virus type 2 genome is involved in amplification of integrated rep-cap sequences” Journal of Virology 75 (20): 9991-9994 (2001); Nony et al., “Evidence for packaging of rep-cap sequences into adeno-associated virus (AAV) type 2 capsids in the absence of inverted terminal repeats: a model for generation of rep-positive AAV particles” Journal of Virology 77 (1): 776-781 (2003); Philpott et al., “Efficient integration of recombinant adeno-associated virus DNA vectors requires a p5-rep sequence in cis” Journal of Virology 76 (11): 5411-5421 (June 2002); and Tullis et al., “Efficient replication of adeno-associated virus type 2 vectors: a cis-acting element outside of the terminal repeats and a minimal size”. Journal of Virology 74 (24): 11511-11521 (2000).

### Pharmaceutical Delivery Systems

The present invention contemplates several delivery systems for PE systems that provide for roughly uniform distribution, have controllable rates of release. A variety of different media are described below that are useful in creating such delivery systems. It is not intended that any one medium or carrier is limiting to the present invention. Note that any medium or carrier may be combined with another medium or carrier.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly (ortho esters), cyanoacrylates, gelatin-resorcin-aldehyde type bioadhesives, polyacrylic acid and copolymers and block copolymers thereof.

### A. Ribonucleoprotein (RNP) Nucleotransfection

In one embodiment, the present invention contemplates mRNA delivery of the PE system. Although it is not necessary to understand the mechanism of an invention, it is believed that delivery of two smaller modular PE mRNAs (e.g., a Cas9/RT mRNA and a pegRNA or petRNA) would improve overall stability and large scale manufacturing efficiency as opposed to full length split PE fusion constructs that are approximately 6-7 kb length. Commercial translation of a full length split PE fusion construct is also problematic due to its small size. Consequently, RNP compositions comprising sPE RNA systems (e.g., nSpy Cas9 RNA+MCP-fused nucleotide polymerase) provides both manufacturing and clinical advantages. In one embodiment, an RNP composition comprising sPE RNA systems are administered using ribonucleotransfection.

To efficiently transport CRISPR-Cas into target tissues/cells require overcoming several extra- and intra-cellular barriers, therefore largely limiting the applications of CRISPR-based therapeutics in vivo. Suggested delivery platforms include, but are not limited to, plasmids, RNAs and ribonucleoproteins (RNPs).

RNPs are composed of a large Cas protein and a short gRNA. gRNA can bind to DNA via Watson-Crick base pairing or the Cas protein can be conjugated to polypeptides, proteins, and PEI. These features can also be used for loading RNP. In addition, RNP can be loaded via electrostatic interactions with positively charged materials due to its negative net charge. These positively charged materials can be cationic lipids, PEI, polypeptides, and metal-organic frameworks (MOFs). Vesicles from cells can also be used to deliver RNP. It has been reported that PEI can coat a complex of Cas9 RNP and DNA nanoclews for enhanced endosomal escape. PEI-coated DNA nanoclews were shown to efficiently transfect a Cas9 RNP targeting EGFP into U2OS cells for EGFP knockout in vitro. Furthermore, the PEI-coated DNA nanoclews could also disrupt EGFP in U2OS.EGFP xenograft tumors in vivo after intratumoral injection. Recently, a nanocapsule was developed for Cas9 RNP delivery. Due to the heterogeneous surface charges of RNP, the RNP was first coated with both cationic and anionic monomers via electrostatic interactions. An imidazole-containing monomer (e.g., glutathione (GSH)-degradable crosslinker) and PEG can be absorbed to the surface of the RNP via hydrogen bonding and van der Waals interactions. Then, GSH-cleavable nanocapsules were formed around the RNP via in situ free-radical polymerization. In addition, targeting ligands, for example CPPs, can be added into the nanocapsule by conjugation to PEG. It was demonstrated that the GSH cleavable nanocapsule could protect Cas9 RNP in the endosome after cellular uptake and could be quickly cleaved by GSH after escape into the cytoplasm for subsequent genome editing. After local injection of Cas9 RNP nanocapsules, robust gene editing was observed in retinal pigment epithelium (RPE) and muscle. Because the net charge of RNP is negative, cationic liposomes or LNPs can be directly used for RNP transfection. It was demonstrated that the Cas9 protein (+22 net charges) can be rendered highly anionic by fusion to a negatively charged GFP (−30 net charges) or complexation with a gRNA. Alternatively, the positively charged PEI has also been developed for RNP delivery. For example, Cas9 RNP was loaded onto GO-PEG-PEI via physisorption and n-stacking interactions. Xu et al., “Rational designs of in vivo CRISPR-Cas delivery systems” Adv Drug Deliv Rev (2021).

RNP delivery for genome editing in live cells may be performed with Lipofectamine® RNAiMAX lipid transfection reagent and elements of a PE system. For example, pegRNAs/petRNAs are mixed with purified Cas9/RT proteins at an equimolar ratio in Opti-MEM™ to from an RNP complex (e.g, −10 min at room temperature). These RNPs can then be transfected into live cells using, for example, DMEM with 10% FBS. RNP nucleotransfection may be performed by electroporation using, for example, a Lonza 96-well Shuttle™ System (Lonza, Basel, Switzerland) optionally in the presence of Alt-R® Cas9 Electroporation Enhancer (Integrated DNA Technologies, Inc). Vakulskas et al., “A high-fidelity Cas9 mutant delivered as a ribonucleoprotein complex enables efficient gene editing in human haematopoietic stem and progenitor cells” Nat Med. 24 (8): 1216-1224 (2018).

The prime editing efficiency of a number of genes was compared between current PE systems and sPE in HEK293T cells using either conventional mRNA delivery or RNP-mediated nucleofection. For example, the genes included FANCF, VEGFA and HEK3.

### B. Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, pseudo-poly(amino acids), polyhydroxybutrate-related copolymers, polyanhydrides, polymethylmethacrylate, poly(ethylene oxide), lecithin and phospholipids.

### C. Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal elements and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### D. Microspheres, Microparticles And Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze-dried medium comprising at least one therapeutic agent is homogenized in a suitable solvent and sprayed to manufacture microspheres in the range of 20 to Techniques are then followed that maintain sustained release integrity during phases of purification, encapsulation and storage. Scott et al., Improving Protein Therapeutics With Sustained Release Formulations, Nature Biotechnology, Volume 16:153-157 (1998). Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA/PGA Copolymer Ratios, J. Biomed. Mater. Res., Vol. II: 711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method. In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-50011M and composed of biocompatible and biodegradable polymers. Specific polymer compositions of a microsphere can control the therapeutic agent release rate such that custom-designed microspheres are possible, including effective management of the burst effect. ProMa® (Epic Therapeutics, Inc.) is a protein-matrix delivery system. The system is aqueous in nature and is adaptable to standard pharmaceutical delivery models. In particular, ProMa® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability.

Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

TABLE 8
Sequences of the full construct
including linkers.

| | |
|---|---|
| PE | MKRTADGSEFESPKKKRKVDKKYSIGLDIGTNSVG |
| (SEQ ID | WAVITDEYKVPSKKFKVLGNTDRHSIKKNLIGALL |
| NO: 35) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLEDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ELDINRLSDYDVDAIVPQSFLKDDSIDNKVLTRSD |
| | KNRGKSDNVPSEEVVKKMKNYWRQLLNAKLITQRK |
| | FDNLTKAERGGLSELDKAGFIKRQLVETRQITKHV |
| | AQILDSRMNTKYDENDKLIREVKVITLKSKLVSDF |
| | RKDFQFYKVREINNYHHAHDAYLNAVVGTALIKKY |
| | PKLESEFVYGDYKVYDVRKMIAKSEQEIGKATAKY |
| | FFYSNIMNFFKTEITLANGEIRKRPLIETNGETGE |
| | IVWDKGRDFATVRKVLSMPQVNIVKKTEVQTGGFS |
| | KESILPKRNSDKLIARKKDWDPKKYGGFDSPTVAY |
| | SVLVVAKVEKGKSKKLKSVKELLGITIMERSSFEK |
| | NPIDFLEAKGYKEVKKDLIIKLPKYSLFELENGRK |
| | RMLASAGELQKGNELALPSKYVNFLYLASHYEKLK |
| | GSPEDNEQKQLFVEQHKHYLDEIIEQISEFSKRVI |
| | LADANLDKVLSAYNKHRDKPIREQAENIIHLFTLT |
| | NLGAPAAFKYFDTTIDRKRYTSTKEVLDATLIHQS |
| | ITGLYETRIDLSQLGGDSGGSSGGSSGSETPGTSE |
| | SATPESSGGSSGGSSTLNIEDEYRLHETSKEPDVS |
| | LGSTWLSDFPQAWAETGGMGLAVRQAPLIIPLKAT |
| | STPVSIKQYPMSQEARLGIKPHIQRLLDQGILVPC |
| | QSPWNTPLLPVKKPGTNDYRPVQDLREVNKRVEDI |
| | HPTVPNPYNLLSGLPPSHQWYTVLDLKDAFFCLRL |
| | HPTSQPLFAFEWRDPEMGISGQLTWTRLPQGFKNS |
| | PTLFNEALHRDLADFRIQHPDLILLQYVDDLLLAA |
| | TSELDCQQGTRALLQTLGNLGYRASAKKAQICQKQ |
| | VKYLGYLLKEGQRWLTEARKETVMGQPTPKTPRQL |
| | REFLGKAGFCRLFIPGFAEMAAPLYPLTKPGTLFN |
| | WGPDQQKAYQEIKQALLTAPALGLPDLTKPFELFV |
| | DEKQGYAKGVLTQKLGPWRRPVAYLSKKLDPVAAG |
| | WPPCLRMVAAIAVLTKDAGKLTMGQPLVILAPHAV |
| | EALVKQPPDRWLSNARMTHYQALLLDTDRVQFGPV |
| | VALNPATLLPLPEEGLQHNCLDILAEAHGTRPDLT |
| | DQPLPDADHTWYTDGSSLLQEGQRKAGAAVTTETE |
| | VIWAKALPAGTSAQRAELIALTQALKMAEGKKLNV |
| | YTDSRYAFATAHIHGEIYRRRGWLTSEGKEIKNKD |
| | EILALLKALFLPKRLSIIHCPGHQKGHSAEARGNR |
| | MADQAARKAAITETPDTSTLLIENSSPSGGSKRTA |
| | DGSEFEPKKKRKV |
| SpyCas9 H840A | MDYKDHDGDYKDHDIDYKDDDDKMAPKKKRKVGIH |
| nickase | GVPAADKKYSIGLDIGTNSVGWAVITDEYKVPSKK |
| (including | FKVLGNTDRHSIKKNLIGALLFDSGETAEATRLKR |
| 3XFLAG, SV40 | TARRRYTRRKNRICYLQEIFSNEMAKVDDSFFHRL |
| NLS, and | EESFLVEEDKKHERHPIFGNIVDEVAYHEKYPTIY |
| nuceloplasmin | HLRKKLVDSTDKADLRLIYLALAHMIKFRGHFLIE |
| NLS + linkers) | GDLNPDNSDVDKLFIQLVQTYNQLFEENPINASGV |
| (SEQ ID NO: | DAKAILSARLSKSRRLENLIAQLPGEKKNGLFGNL |
| 36) | IALSLGLTPNFKSNFDLAEDAKLQLSKDTYDDDLD |
| | NLLAQIGDQYADLFLAAKNLSDAILLSDILRVNTE |
| | ITKAPLSASMIKRYDEHHQDLTLLKALVRQQLPEK |
| | YKEIFFDQSKNGYAGYIDGGASQEEFYKFIKPILE |
| | KMDGTEELLVKLNREDLLRKQRTFDNGSIPHQIHL |
| | GELHAILRRQEDFYPFLKDNREKIEKILTFRIPVG |
| | PLARGNSRFAWMTRKSEETITPWNFEEVVDKGASA |
| | QSFIERMTNFDKNLPNEKVLPKHSLLYEYFTVYNE |
| | LTKVKYVTEGMRKPAFLSGEQKKAIVDLLFKTNRK |
| | VTVKQLKEDYFKKIECFDSVEISGVEDRFNASLGT |
| | YHDLLKIIKDKDFLDNEENEDILEDIVLTLTLFED |
| | REMIEERLKTYAHLFDDKVMKQLKRRRYTGWGRLS |
| | RKLINGIRDKQSGKTILDFLKSDGFANRNFMQLIH |
| | DDSLTFKEDIQKAQVSGQGDSLHEHIANLAGSPAI |
| | KKGILQTVKVVDELVKVMGRHKPENIVIEMARENQ |
| | TTQKGQKNSRERMKRIEEGIKELGSQILKEHPVEN |
| | TQLQNEKLYLLQNGRDMYVDQELDINRLSDYDVDA |
| | IVPQSFLKDDSIDNKVLTRSDKNRGKSDNVPSEEV |
| | VKKMKNYWRQLLNAKLITQRKFDNLTKAERGGLSE |
| | LDKAGFIKRQLVETRQITKHVAQILDSRMNTKYDE |
| | NDKLIREVKVITLKSKLVSDFRKDFQFYKVREINN |
| | YHHAHDAYLNAVVGTALIKKYPKLESEFVYGDYKV |
| | YDVRKMIAKSEQEIGKATAKYFFYSNIMNFFKTEI |
| | TLANGEIRKRPLIETNGETGEIVWDKGRDFATVRK |
| | VLSMPQVNIVKKTEVQTGGFSKESILPKRNSDKLI |
| | ARKKDWDPKKYGGFDSPTVAYSVLVVAKVEKGKSK |
| | KLKSVKELLGITIMERSSFEKNPIDFLEAKGYKEV |
| | KKDLIIKLPKYSLFELENGRKRMLASAGELQKGNE |
| | LALPSKYVNFLYLASHYEKLKGSPEDNEQKQLFVE |
| | QHKHYLDEIIEQISEFSKRVILADANLDKVLSAYN |
| | KHRDKPIREQAENIIHLFTLTNLGAPAAFKYFDTT |
| | IDRKRYTSTKEVLDATLIHQSITGLYETRIDLSQL |
| | GGDKRPAATKKAGQAKKKK |
| MCP-RT | MASNFTQFVLVDNGGTGDVTVAPSNFANGVAEWIS |
| (including | SNSRSQAYKVTCSVRQSSAQKRKYTIKVEVPKVAT |
| SV40 | QTVGGVELPVAAWRSYLNMELTIPIFATNSDCELI |
| NLS + linker) | VKAMQGLLKDGNPIPSAIAANSGIYSGGSSGGSSG |
| (SEQ ID NO: | SETPGTSESATPESSGGSSGGSSTLNIEDEYRLHE |
| 37) | TSKEPDVSLGSTWLSDFPQAWAETGGMGLAVRQAP |
| | LIIPLKATSTPVSIKQYPMSQEARLGIKPHIQRLL |
| | DQGILVPCQSPWNTPLLPVKKPGTNDYRPVQDLRE |
| | VNKRVEDIHPTVPNPYNLLSGLPPSHQWYTVLDLK |
| | DAFFCLRLHPTSQPLFAFEWRDPEMGISGQLTWTR |
| | LPQGFKNSPTLFNEALHRDLADFRIQHPDLILLQY |
| | VDDLLLAATSELDCQQGTRALLQTLGNLGYRASAK |
| | KAQICQKQVKYLGYLLKEGQRWLTEARKETVMGQP |
| | TPKTPRQLREFLGKAGFCRLFIPGFAEMAAPLYPL |
| | TKPGTLFNWGPDQQKAYQEIKQALLTAPALGLPDL |
| | TKPFELFVDEKQGYAKGVLTQKLGPWRRPVAYLSK |
| | KLDPVAAGWPPCLRMVAAIAVLTKDAGKLTMGQPL |
| | VILAPHAVEALVKQPPDRWLSNARMTHYQALLLDT |
| | DRVQFGPVVALNPATLLPLPEEGLQHNCLDILAEA |
| | HGTRPDLTDQPLPDADHTWYTDGSSLLQEGQRKAG |
| | AAVTTETEVIWAKALPAGTSAQRAELIALTQALKM |
| | AEGKKLNVYTDSRYAFATAHIHGEIYRRRGWLTSE |
| | GKEIKNKDEILALLKALFLPKRLSIIHCPGHQKGH |
| | SAEARGNRMADQAARKAAITETPDTSTLLIENSSP |
| | SGGSKRTADGSEFEPKKKRKV |
| mM-PE | MKRTADGSEFESPKKKRKVDKKYSIGLDIGTNSVG |
| (SEQ ID NO: | WAVITDEYKVPSKKFKVLGNTDRHSIKKNLIGALL |
| 38) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ELDINRLSDYDVDAIVPQSFLKDDSIDNKVLTRSD |
| | KNRGKSDNVPSEEVVKKMKNYWRQLLNAKLITQRK |
| | FDNLTKAERGGLSELDKAGFIKRQLVETRQITKHV |
| | AQILDSRMNTKYDENDKLIREVKVITLKSKLVSDF |
| | RKDFQFYKVREINNYHHAHDAYLNAVVGTALIKKY |
| | PKLESEFVYGDYKVYDVRKMIAKSEQEIGKATAKY |
| | FFYSNIMNFFKTEITLANGEIRKRPLIETNGETGE |
| | IVWDKGRDFATVRKVLSMPQVNIVKKTEVQTGGFS |
| | KESILPKRNSDKLIARKKDWDPKKYGGFDSPTVAY |
| | SVLVVAKVEKGKSKKLKSVKELLGITIMERSSFEK |
| | NPIDFLEAKGYKEVKKDLIIKLPKYSLFELENGRK |
| | RMLASAGELQKGNELALPSKYVNFLYLASHYEKLK |
| | GSPEDNEQKQLFVEQHKHYLDEIIEQISEFSKRVI |
| | LADANLDKVLSAYNKHRDKPIREQAENIIHLFTLT |
| | NLGAPAAFKYFDTTIDRKRYTSTKEVLDATLIHQS |
| | ITGLYETRIDLSQLGGDSGGSSGGSSGSETPGTSE |
| | SATPESSGGSSGGSSASNFTQFVLVDNGGTGDVTV |
| | APSNFANGVAEWISSNSRSQAYKVTCSVRQSSAQK |
| | RKYTIKVEVPKVATQTVGGVELPVAAWRSYLNMEL |
| | TIPIFATNSDCELIVKAMQGLLKDGNPIPSAIAAN |
| | SGIYSGGSSGGSSGSETPGTSESATPESSGGSSGG |
| | SSTLNIEDEYRLHETSKEPDVSLGSTWLSDFPQAW |
| | AETGGMGLAVRQAPLIIPLKATSTPVSIKQYPMSQ |
| | EARLGIKPHIQRLLDQGILVPCQSPWNTPLLPVKK |
| | PGTNDYRPVQDLREVNKRVEDIHPTVPNPYNLLSG |
| | LPPSHQWYTVLDLKDAFFCLRLHPTSQPLFAFEWR |
| | DPEMGISGQLTWTRLPQGFKNSPTLFNEALHRDLA |
| | DFRIQHPDLILLQYVDDLLLAATSELDCQQGTRAL |
| | LQTLGNLGYRASAKKAQICQKQVKYLGYLLKEGQR |
| | WLTEARKETVMGQPTPKTPRQLREFLGKAGFCRLF |
| | IPGFAEMAAPLYPLTKPGTLFNWGPDQQKAYQEIK |
| | QALLTAPALGLPDLTKPFELFVDEKQGYAKGVLTQ |
| | KLGPWRRPVAYLSKKLDPVAAGWPPCLRMVAAIAV |
| | LTKDAGKLTMGQPLVILAPHAVEALVKQPPDRWLS |
| | NARMTHYQALLLDTDRVQFGPVVALNPATLLPLPE |
| | EGLQHNCLDILAEAHGTRPDLTDQPLPDADHTWYT |
| | DGSSLLQEGQRKAGAAVTTETEVIWAKALPAGTSA |
| | QRAELIALTQALKMAEGKKLNVYTDSRYAFATAHI |
| | HGEIYRRRGWLTSEGKEIKNKDEILALLKALFLPK |
| | RLSIIHCPGHQKGHSAEARGNRMADQAARKAAITE |
| | TPDTSTLLIENSSPSGGSKRTADGSEFEPKKKRKV |
| CM-PE | MKRTADGSEFESPKKKRKVDKKYSIGLDIGTNSVG |
| (SEQ ID | WAVITDEYKVPSKKFKVLGNTDRHSIKKNLIGALL |
| NO: 39) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ELDINRLSDYDVDAIVPQSFLKDDSIDNKVLTRSD |
| | KNRGKSDNVPSEEVVKKMKNYWRQLLNAKLITQRK |
| | FDNLTKAERGGLSELDKAGFIKRQLVETRQITKHV |
| | AQILDSRMNTKYDENDKLIREVKVITLKSKLVSDF |
| | RKDFQFYKVREINNYHHAHDAYLNAVVGTALIKKY |
| | PKLESEFVYGDYKVYDVRKMIAKSEQEIGKATAKY |
| | FFYSNIMNFFKTEITLANGEIRKRPLIETNGETGE |
| | IVWDKGRDFATVRKVLSMPQVNIVKKTEVQTGGFS |
| | KESILPKRNSDKLIARKKDWDPKKYGGFDSPTVAY |
| | SVLVVAKVEKGKSKKLKSVKELLGITIMERSSFEK |
| | NPIDFLEAKGYKEVKKDLIIKLPKYSLFELENGRK |
| | RMLASAGELQKGNELALPSKYVNFLYLASHYEKLK |
| | GSPEDNEQKQLFVEQHKHYLDEIIEQISEFSKRVI |
| | LADANLDKVLSAYNKHRDKPIREQAENIIHLFTLT |
| | NLGAPAAFKYFDTTIDRKRYTSTKEVLDATLIHQS |
| | ITGLYETRIDLSQLGGDSGGSSGGSSGSETPGTSE |
| | SATPESSGGSSGGSSTLNIEDEYRLHETSKEPDVS |
| | LGSTWLSDFPQAWAETGGMGLAVRQAPLIIPLKAT |
| | STPVSIKQYPMSQEARLGIKPHIQRLLDQGILVPC |
| | QSPWNTPLLPVKKPGTNDYRPVQDLREVNKRVEDI |
| | HPTVPNPYNLLSGLPPSHQWYTVLDLKDAFFCLRL |
| | HPTSQPLFAFEWRDPEMGISGQLTWTRLPQGFKNS |
| | PTLFNEALHRDLADFRIQHPDLILLQYVDDLLLAA |
| | TSELDCQQGTRALLQTLGNLGYRASAKKAQICQKQ |
| | VKYLGYLLKEGQRWLTEARKETVMGQPTPKTPRQL |
| | REFLGKAGFCRLFIPGFAEMAAPLYPLTKPGTLFN |
| | WGPDQQKAYQEIKQALLTAPALGLPDLTKPFELFV |
| | DEKQGYAKGVLTQKLGPWRRPVAYLSKKLDPVAAG |
| | WPPCLRMVAAIAVLTKDAGKLTMGQPLVILAPHAV |
| | EALVKQPPDRWLSNARMTHYQALLLDTDRVQFGPV |
| | VALNPATLLPLPEEGLQHNCLDILAEAHGTRPDLT |
| | DQPLPDADHTWYTDGSSLLQEGQRKAGAAVTTETE |
| | VIWAKALPAGTSAQRAELIALTQALKMAEGKKLNV |
| | YTDSRYAFATAHIHGEIYRRRGWLTSEGKEIKNKD |
| | EILALLKALFLPKRLSIIHCPGHQKGHSAEARGNR |
| | MADQAARKAAITETPDTSTLLIENSSPSGGSSGGS |
| | SGSETPGTSESATPESSGGSSGGSSASNFTQFVLV |
| | DNGGTGDVTVAPSNFANGVAEWISSNSRSQAYKVT |
| | CSVRQSSAQKRKYTIKVEVPKVATQTVGGVELPVA |
| | AWRSYLNMELTIPIFATNSDCELIVKAMQGLLKDG |
| | NPIPSAIAANSGIYSGGSKRTADGSEFEPKKKRKV |
| nM-PE | MKRTADGSEFESPKKKRKVASNFTQFVLVDNGGTG |
| (SEQ ID | DVTVAPSNFANGVAEWISSNSRSQAYKVTCSVRQS |
| NO: 40) | SAQKRKYTIKVEVPKVATQTVGGVELPVAAWRSYL |
| | NMELTIPIFATNSDCELIVKAMQGLLKDGNPIPSA |
| | IAANSGIYSAGGGGSGGGGSGGGGSGPKKKRKVAA |
| | AGSDKKYSIGLDIGTNSVGWAVITDEYKVPSKKFK |
| | VLGNTDRHSIKKNLIGALLFDSGETAEATRLKRTA |
| | RRRYTRRKNRICYLQEIFSNEMAKVDDSFFHRLEE |
| | SFLVEEDKKHERHPIFGNIVDEVAYHEKYPTIYHL |
| | RKKLVDSTDKADLRLIYLALAHMIKFRGHFLIEGD |
| | LNPDNSDVDKLFIQLVQTYNQLFEENPINASGVDA |
| | KAILSARLSKSRRLENLIAQLPGEKKNGLFGNLIA |
| | LSLGLTPNFKSNFDLAEDAKLQLSKDTYDDDLDNL |
| | LAQIGDQYADLFLAAKNLSDAILLSDILRVNTEIT |
| | KAPLSASMIKRYDEHHQDLTLLKALVRQQLPEKYK |
| | EIFFDQSKNGYAGYIDGGASQEEFYKFIKPILEKM |
| | DGTEELLVKLNREDLLRKQRTFDNGSIPHQIHLGE |
| | LHAILRRQEDFYPFLKDNREKIEKILTFRIPVGPL |
| | ARGNSRFAWMTRKSEETITPWNFEEVVDKGASAQS |
| | FIERMTNFDKNLPNEKVLPKHSLLYEYFTVYNELT |
| | KVKYVTEGMRKPAFLSGEQKKAIVDLLFKTNRKVT |
| | VKQLKEDYFKKIECFDSVEISGVEDRFNASLGTYH |
| | DLLKIIKDKDFLDNEENEDILEDIVLTLTLFEDRE |
| | MIEERLKTYAHLFDDKVMKQLKRRRYTGWGRLSRK |
| | LINGIRDKQSGKTILDFLKSDGFANRNFMQLIHDD |
| | SLTFKEDIQKAQVSGQGDSLHEHIANLAGSPAIKK |
| | GILQTVKVVDELVKVMGRHKPENIVIEMARENQTT |
| | QKGQKNSRERMKRIEEGIKELGSQILKEHPVENTQ |
| | LQNEKLYLLQNGRDMYVDQELDINRLSDYDVDAIV |
| | PQSFLKDDSIDNKVLTRSDKNRGKSDNVPSEEVVK |
| | KMKNYWRQLLNAKLITQRKFDNLTKAERGGLSELD |
| | KAGFIKRQLVETRQITKHVAQILDSRMNTKYDEND |
| | KLIREVKVITLKSKLVSDFRKDFQFYKVREINNYH |
| | HAHDAYLNAVVGTALIKKYPKLESEFVYGDYKVYD |
| | VRKMIAKSEQEIGKATAKYFFYSNIMNFFKTEITL |
| | ANGEIRKRPLIETNGETGEIVWDKGRDFATVRKVL |
| | SMPQVNIVKKTEVQTGGFSKESILPKRNSDKLIAR |
| | KKDWDPKKYGGFDSPTVAYSVLVVAKVEKGKSKKL |
| | KSVKELLGITIMERSSFEKNPIDFLEAKGYKEVKK |
| | DLIIKLPKYSLFELENGRKRMLASAGELQKGNELA |
| | LPSKYVNFLYLASHYEKLKGSPEDNEQKQLFVEQH |
| | KHYLDEIIEQISEFSKRVILADANLDKVLSAYNKH |
| | RDKPIREQAENIIHLFTLTNLGAPAAFKYFDTTID |
| | RKRYTSTKEVLDATLIHQSITGLYETRIDLSQLGG |
| | DSGGSSGGSSGSETPGTSESATPESSGGSSGGSST |
| | LNIEDEYRLHETSKEPDVSLGSTWLSDFPQAWAET |
| | GGMGLAVRQAPLIIPLKATSTPVSIKQYPMSQEAR |
| | LGIKPHIQRLLDQGILVPCQSPWNTPLLPVKKPGT |
| | NDYRPVQDLREVNKRVEDIHPTVPNPYNLLSGLPP |
| | SHQWYTVLDLKDAFFCLRLHPTSQPLFAFEWRDPE |
| | MGISGQLTWTRLPQGFKNSPTLFNEALHRDLADFR |
| | IQHPDLILLQYVDDLLLAATSELDCQQGTRALLQT |
| | LGNLGYRASAKKAQICQKQVKYLGYLLKEGQRWLT |
| | EARKETVMGQPTPKTPRQLREFLGKAGFCRLFIPG |
| | FAEMAAPLYPLTKPGTLFNWGPDQQKAYQEIKQAL |
| | LTAPALGLPDLTKPFELFVDEKQGYAKGVLTQKLG |
| | PWRRPVAYLSKKLDPVAAGWPPCLRMVAAIAVLTK |
| | DAGKLTMGQPLVILAPHAVEALVKQPPDRWLSNAR |
| | MTHYQALLLDTDRVQFGPVVALNPATLLPLPEEGL |
| | QHNCLDILAEAHGTRPDLTDQPLPDADHTWYTDGS |
| | SLLQEGQRKAGAAVTTETEVIWAKALPAGTSAQRA |
| | ELIALTQALKMAEGKKLNVYTDSRYAFATAHIHGE |
| | IYRRRGWLTSEGKEIKNKDEILALLKALFLPKRLS |
| | IIHCPGHQKGHSAEARGNRMADQAARKAAITETPD |
| | TSTLLIENSSPSGGSKRTADGSEFEPKKKRKV |
| nMM-PE | MKRTADGSEFESPKKKRKVASNFTQFVLVDNGGTG |
| (SEQ ID | DVTVAPSNFANGVAEWISSNSRSQAYKVTCSVRQS |
| NO: 41) | SAQKRKYTIKVEVPKVATQTVGGVELPVAAWRSYL |
| | NMELTIPIFATNSDCELIVKAMQGLLKDGNPIPSA |
| | IAANSGIYGSSGSETPGTSESATPESSGASNFTQF |
| | VLVDNGGTGDVTVAPSNFANGVAEWISSNSRSQAY |
| | KVTCSVRQSSAQKRKYTIKVEVPKVATQTVGGVEL |
| | PVAAWRSYLNMELTIPIFATNSDCELIVKAMQGLL |
| | KDGNPIPSAIAANSGIYSAGGGGSGGGGSGGGGSG |
| | PKKKRKVAAAGSDKKYSIGLDIGTNSVGWAVITDE |
| | YKVPSKKFKVLGNTDRHSIKKNLIGALLFDSGETA |
| | EATRLKRTARRRYTRRKNRICYLQEIFSNEMAKVD |
| | DSFFHRLEESFLVEEDKKHERHPIFGNIVDEVAYH |
| | EKYPTIYHLRKKLVDSTDKADLRLIYLALAHMIKF |
| | RGHFLIEGDLNPDNSDVDKLFIQLVQTYNQLFEEN |
| | PINASGVDAKAILSARLSKSRRLENLIAQLPGEKK |
| | NGLFGNLIALSLGLTPNFKSNFDLAEDAKLQLSKD |
| | TYDDDLDNLLAQIGDQYADLFLAAKNLSDAILLSD |
| | ILRVNTEITKAPLSASMIKRYDEHHQDLTLLKALV |
| | RQQLPEKYKEIFFDQSKNGYAGYIDGGASQEEFYK |
| | FIKPILEKMDGTEELLVKLNREDLLRKQRTFDNGS |
| | IPHQIHLGELHAILRRQEDFYPFLKDNREKIEKIL |
| | TFRIPVGPLARGNSRFAWMTRKSEETITPWNFEEV |
| | VDKGASAQSFIERMTNFDKNLPNEKVLPKHSLLYE |
| | YFTVYNELTKVKYVTEGMRKPAFLSGEQKKAIVDL |
| | LFKTNRKVTVKQLKEDYFKKIECFDSVEISGVEDR |
| | FNASLGTYHDLLKIIKDKDFLDNEENEDILEDIVL |
| | TLTLFEDREMIEERLKTYAHLFDDKVMKQLKRRRY |
| | TGWGRLSRKLINGIRDKQSGKTILDFLKSDGFANR |
| | NFMQLIHDDSLTFKEDIQKAQVSGQGDSLHEHIAN |
| | LAGSPAIKKGILQTVKVVDELVKVMGRHKPENIVI |
| | EMARENQTTQKGQKNSRERMKRIEEGIKELGSQIL |
| | KEHPVENTQLQNEKLYLLQNGRDMYVDQELDINRL |
| | SDYDVDAIVPQSFLKDDSIDNKVLTRSDKNRGKSD |
| | NVPSEEVVKKMKNYWRQLLNAKLITQRKFDNLTKA |
| | ERGGLSELDKAGFIKRQLVETRQITKHVAQILDSR |
| | MNTKYDENDKLIREVKVITLKSKLVSDFRKDFQFY |
| | KVREINNYHHAHDAYLNAVVGTALIKKYPKLESEF |
| | VYGDYKVYDVRKMIAKSEQEIGKATAKYFFYSNIM |
| | NFFKTEITLANGEIRKRPLIETNGETGEIVWDKGR |
| | DFATVRKVLSMPQVNIVKKTEVQTGGFSKESILPK |
| | RNSDKLIARKKDWDPKKYGGFDSPTVAYSVLVVAK |
| | VEKGKSKKLKSVKELLGITIMERSSFEKNPIDFLE |
| | AKGYKEVKKDLIIKLPKYSLFELENGRKRMLASAG |
| | ELQKGNELALPSKYVNFLYLASHYEKLKGSPEDNE |
| | QKQLFVEQHKHYLDEIIEQISEFSKRVILADANLD |
| | KVLSAYNKHRDKPIREQAENIIHLFTLTNLGAPAA |
| | FKYFDTTIDRKRYTSTKEVLDATLIHQSITGLYET |
| | RIDLSQLGGDSGGSSGGSSGSETPGTSESATPESS |
| | GGSSGGSSTLNIEDEYRLHETSKEPDVSLGSTWLS |
| | DFPQAWAETGGMGLAVRQAPLIIPLKATSTPVSIK |
| | QYPMSQEARLGIKPHIQRLLDQGILVPCQSPWNTP |
| | LLPVKKPGTNDYRPVQDLREVNKRVEDIHPTVPNP |
| | YNLLSGLPPSHQWYTVLDLKDAFFCLRLHPTSQPL |
| | FAFEWRDPEMGISGQLTWTRLPQGFKNSPTLFNEA |
| | LHRDLADFRIQHPDLILLQYVDDLLLAATSELDCQ |
| | QGTRALLQTLGNLGYRASAKKAQICQKQVKYLGYL |
| | LKEGQRWLTEARKETVMGQPTPKTPRQLREFLGKA |
| | GFCRLFIPGFAEMAAPLYPLTKPGTLFNWGPDQQK |
| | AYQEIKQALLTAPALGLPDLTKPFELFVDEKQGYA |
| | KGVLTQKLGPWRRPVAYLSKKLDPVAAGWPPCLRM |
| | VAAIAVLTKDAGKLTMGQPLVILAPHAVEALVKQP |
| | PDRWLSNARMTHYQALLLDTDRVQFGPVVALNPAT |
| | LLPLPEEGLQHNCLDILAEAHGTRPDLTDQPLPDA |
| | DHTWYTDGSSLLQEGQRKAGAAVTTETEVIWAKAL |
| | PAGTSAQRAELIALTQALKMAEGKKLNVYTDSRYA |
| | FATAHIHGEIYRRRGWLTSEGKEIKNKDEILALLK |
| | ALFLPKRLSIIHCPGHQKGHSAEARGNRMADQAAR |
| | KAAITETPDTSTLLIENSSPSGGSKRTADGSEFEP |
| | KKKRKV |
| nMcM-PE | MKRTADGSEFESPKKKRKVASNFTQFVLVDNGGTG |
| (SEQ ID | DVTVAPSNFANGVAEWISSNSRSQAYKVTCSVRQS |
| NO: 42) | SAQKRKYTIKVEVPKVATQTVGGVELPVAAWRSYL |
| | NMELTIPIFATNSDCELIVKAMQGLLKDGNPIPSA |
| | IAANSGIYSAGGGGSGGGGSGGGGSGPKKKRKVAA |
| | AGSDKKYSIGLDIGTNSVGWAVITDEYKVPSKKFK |
| | VLGNTDRHSIKKNLIGALLFDSGETAEATRLKRTA |
| | RRRYTRRKNRICYLQEIFSNEMAKVDDSFFHRLEE |
| | SFLVEEDKKHERHPIFGNIVDEVAYHEKYPTIYHL |
| | RKKLVDSTDKADLRLIYLALAHMIKFRGHFLIEGD |
| | LNPDNSDVDKLFIQLVQTYNQLFEENPINASGVDA |
| | KAILSARLSKSRRLENLIAQLPGEKKNGLFGNLIA |
| | LSLGLTPNFKSNFDLAEDAKLQLSKDTYDDDLDNL |
| | LAQIGDQYADLFLAAKNLSDAILLSDILRVNTEIT |
| | KAPLSASMIKRYDEHHQDLTLLKALVRQQLPEKYK |
| | EIFFDQSKNGYAGYIDGGASQEEFYKFIKPILEKM |
| | DGTEELLVKLNREDLLRKQRTFDNGSIPHQIHLGE |
| | LHAILRRQEDFYPFLKDNREKIEKILTFRIPVGPL |
| | ARGNSRFAWMTRKSEETITPWNFEEVVDKGASAQS |
| | FIERMTNFDKNLPNEKVLPKHSLLYEYFTVYNELT |
| | KVKYVTEGMRKPAFLSGEQKKAIVDLLFKTNRKVT |
| | VKQLKEDYFKKIECFDSVEISGVEDRFNASLGTYH |
| | DLLKIIKDKDFLDNEENEDILEDIVLTLTLFEDRE |
| | MIEERLKTYAHLFDDKVMKQLKRRRYTGWGRLSRK |
| | LINGIRDKQSGKTILDFLKSDGFANRNFMQLIHDD |
| | SLTFKEDIQKAQVSGQGDSLHEHIANLAGSPAIKK |
| | GILQTVKVVDELVKVMGRHKPENIVIEMARENQTT |
| | QKGQKNSRERMKRIEEGIKELGSQILKEHPVENTQ |
| | LQNEKLYLLQNGRDMYVDQELDINRLSDYDVDAIV |
| | PQSFLKDDSIDNKVLTRSDKNRGKSDNVPSEEVVK |
| | KMKNYWRQLLNAKLITQRKFDNLTKAERGGLSELD |
| | KAGFIKRQLVETRQITKHVAQILDSRMNTKYDEND |
| | KLIREVKVITLKSKLVSDFRKDFQFYKVREINNYH |
| | HAHDAYLNAVVGTALIKKYPKLESEFVYGDYKVYD |
| | VRKMIAKSEQEIGKATAKYFFYSNIMNFFKTEITL |
| | ANGEIRKRPLIETNGETGEIVWDKGRDFATVRKVL |
| | SMPQVNIVKKTEVQTGGFSKESILPKRNSDKLIAR |
| | KKDWDPKKYGGFDSPTVAYSVLVVAKVEKGKSKKL |
| | KSVKELLGITIMERSSFEKNPIDFLEAKGYKEVKK |
| | DLIIKLPKYSLFELENGRKRMLASAGELQKGNELA |
| | LPSKYVNFLYLASHYEKLKGSPEDNEQKQLFVEQH |
| | KHYLDEIIEQISEFSKRVILADANLDKVLSAYNKH |
| | RDKPIREQAENIIHLFTLTNLGAPAAFKYFDTTID |
| | RKRYTSTKEVLDATLIHQSITGLYETRIDLSQLGG |
| | DSGGSSGGSSGSETPGTSESATPESSGGSSGGSST |
| | LNIEDEYRLHETSKEPDVSLGSTWLSDFPQAWAET |
| | GGMGLAVRQAPLIIPLKATSTPVSIKQYPMSQEAR |
| | LGIKPHIQRLLDQGILVPCQSPWNTPLLPVKKPGT |
| | NDYRPVQDLREVNKRVEDIHPTVPNPYNLLSGLPP |
| | SHQWYTVLDLKDAFFCLRLHPTSQPLFAFEWRDPE |
| | MGISGQLTWTRLPQGFKNSPTLFNEALHRDLADFR |
| | IQHPDLILLQYVDDLLLAATSELDCQQGTRALLQT |
| | LGNLGYRASAKKAQICQKQVKYLGYLLKEGQRWLT |
| | EARKETVMGQPTPKTPRQLREFLGKAGFCRLFIPG |
| | FAEMAAPLYPLTKPGTLFNWGPDQQKAYQEIKQAL |
| | LTAPALGLPDLTKPFELFVDEKQGYAKGVLTQKLG |
| | PWRRPVAYLSKKLDPVAAGWPPCLRMVAAIAVLTK |
| | DAGKLTMGQPLVILAPHAVEALVKQPPDRWLSNAR |
| | MTHYQALLLDTDRVQFGPVVALNPATLLPLPEEGL |
| | QHNCLDILAEAHGTRPDLTDQPLPDADHTWYTDGS |
| | SLLQEGQRKAGAAVTTETEVIWAKALPAGTSAQRA |
| | ELIALTQALKMAEGKKLNVYTDSRYAFATAHIHGE |
| | IYRRRGWLTSEGKEIKNKDEILALLKALFLPKRLS |
| | IIHCPGHQKGHSAEARGNRMADQAARKAAITETPD |
| | TSTLLIENSSPSGGSSGGSSGSETPGTSESATPES |
| | SGGSSGGSSASNFTQFVLVDNGGTGDVTVAPSNFA |
| | NGVAEWISSNSRSQAYKVTCSVRQSSAQKRKYTIK |
| | VEVPKVATQTVGGVELPVAAWRSYLNMELTIPIFA |
| | TNSDCELIVKAMQGLLKDGNPIPSAIAANSGIYSG |
| | GSKRTADGSEFEPKKKRKV |
| iM-S355-PE | MKRTADGSEFESPKKKRKVDKKYSIGLDIGTNSVG |
| (SEQ ID | WAVITDEYKVPSKKFKVLGNTDRHSIKKNLIGALL |
| NO: 43) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSGSSGSETPGTSE |
| | SATPESSGASNFTQFVLVDNGGTGDVTVAPSNFAN |
| | GVAEWISSNSRSQAYKVTCSVRQSSAQKRKYTIKV |
| | EVPKVATQTVGGVELPVAAWRSYLNMELTIPIFAT |
| | NSDCELIVKAMQGLLKDGNPIPSAIAANSGIYGGS |
| | GGSGGSGGSGGSGGSGGKNGYAGYIDGGASQEEFY |
| | KFIKPILEKMDGTEELLVKLNREDLLRKQRTFDNG |
| | SIPHQIHLGELHAILRRQEDFYPFLKDNREKIEKI |
| | LTFRIPVGPLARGNSRFAWMTRKSEETITPWNFEE |
| | VVDKGASAQSFIERMTNFDKNLPNEKVLPKHSLLY |
| | EYFTVYNELTKVKYVTEGMRKPAFLSGEQKKAIVD |
| | LLFKTNRKVTVKQLKEDYFKKIECFDSVEISGVED |
| | RFNASLGTYHDLLKIIKDKDFLDNEENEDILEDIV |
| | LTLTLFEDREMIEERLKTYAHLFDDKVMKQLKRRR |
| | YTGWGRLSRKLINGIRDKQSGKTILDFLKSDGFAN |
| | RNFMQLIHDDSLTFKEDIQKAQVSGQGDSLHEHIA |
| | NLAGSPAIKKGILQTVKVVDELVKVMGRHKPENIV |
| | IEMARENQTTQKGQKNSRERMKRIEEGIKELGSQI |
| | LKEHPVENTQLQNEKLYLLQNGRDMYVDQELDINR |
| | LSDYDVDAIVPQSFLKDDSIDNKVLTRSDKNRGKS |
| | DNVPSEEVVKKMKNYWRQLLNAKLITQRKFDNLTK |
| | AERGGLSELDKAGFIKRQLVETRQITKHVAQILDS |
| | RMNTKYDENDKLIREVKVITLKSKLVSDFRKDFQF |
| | YKVREINNYHHAHDAYLNAVVGTALIKKYPKLESE |
| | FVYGDYKVYDVRKMIAKSEQEIGKATAKYFFYSNI |
| | MNFFKTEITLANGEIRKRPLIETNGETGEIVWDKG |
| | RDFATVRKVLSMPQVNIVKKTEVQTGGFSKESILP |
| | KRNSDKLIARKKDWDPKKYGGFDSPTVAYSVLVVA |
| | KVEKGKSKKLKSVKELLGITIMERSSFEKNPIDFL |
| | EAKGYKEVKKDLIIKLPKYSLFELENGRKRMLASA |
| | GELQKGNELALPSKYVNFLYLASHYEKLKGSPEDN |
| | EQKQLFVEQHKHYLDEIIEQISEFSKRVILADANL |
| | DKVLSAYNKHRDKPIREQAENIIHLFTLTNLGAPA |
| | AFKYFDTTIDRKRYTSTKEVLDATLIHQSITGLYE |
| | TRIDLSQLGGDSGGSSGGSSGSETPGTSESATPES |
| | SGGSSGGSSTLNIEDEYRLHETSKEPDVSLGSTWL |
| | SDFPQAWAETGGMGLAVRQAPLIIPLKATSTPVSI |
| | KQYPMSQEARLGIKPHIQRLLDQGILVPCQSPWNT |
| | PLLPVKKPGTNDYRPVQDLREVNKRVEDIHPTVPN |
| | PYNLLSGLPPSHQWYTVLDLKDAFFCLRLHPTSQP |
| | LFAFEWRDPEMGISGQLTWTRLPQGFKNSPTLFNE |
| | ALHRDLADFRIQHPDLILLQYVDDLLLAATSELDC |
| | QQGTRALLQTLGNLGYRASAKKAQICQKQVKYLGY |
| | LLKEGQRWLTEARKETVMGQPTPKTPRQLREFLGK |
| | AGFCRLFIPGFAEMAAPLYPLTKPGTLFNWGPDQQ |
| | KAYQEIKQALLTAPALGLPDLTKPFELFVDEKQGY |
| | AKGVLTQKLGPWRRPVAYLSKKLDPVAAGWPPCLR |
| | MVAAIAVLTKDAGKLTMGQPLVILAPHAVEALVKQ |
| | PPDRWLSNARMTHYQALLLDTDRVQFGPVVALNPA |
| | TLLPLPEEGLQHNCLDILAEAHGTRPDLTDQPLPD |
| | ADHTWYTDGSSLLQEGQRKAGAAVTTETEVIWAKA |
| | LPAGTSAQRAELIALTQALKMAEGKKLNVYTDSRY |
| | AFATAHIHGEIYRRRGWLTSEGKEIKNKDEILALL |
| | KALFLPKRLSIIHCPGHQKGHSAEARGNRMADQAA |
| | RKAAITETPDTSTLLIENSSPSGGSKRTADGSEFE |
| | PKKKRKV |
| iMM-E1026-PE | MKRTADGSEFESPKKKRKVDKKYSIGLDIGTNSVG |
| (SEQ ID | WAVITDEYKVPSKKFKVLGNTDRHSIKKNLIGALL |
| NO: 44) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ELDINRLSDYDVDAIVPQSFLKDDSIDNKVLTRSD |
| | KNRGKSDNVPSEEVVKKMKNYWRQLLNAKLITQRK |
| | FDNLTKAERGGLSELDKAGFIKRQLVETRQITKHV |
| | AQILDSRMNTKYDENDKLIREVKVITLKSKLVSDF |
| | RKDFQFYKVREINNYHHAHDAYLNAVVGTALIKKY |
| | PKLESEFVYGDYKVYDVRKMIAKSESGGSSGGSSG |
| | GSSGGSASNFTQFVLVDNGGTGDVTVAPSNFANGV |
| | AEWISSNSRSQAYKVTCSVRQSSAQKRKYTIKVEV |
| | PKVATQTVGGVELPVAAWRSYLNMELTIPIFATNS |
| | DCELIVKAMQGLLKDGNPIPSAIAANSGIYGSSGS |
| | ETPGTSESATPESSGASNFTQFVLVDNGGTGDVTV |
| | APSNFANGVAEWISSNSRSQAYKVTCSVRQSSAQK |
| | RKYTIKVEVPKVATQTVGGVELPVAAWRSYLNMEL |
| | TIPIFATNSDCELIVKAMQGLLKDGNPIPSAIAAN |
| | SGIYSAGGGGSGGGGSGGGGSGQEIGKATAKYFFY |
| | SNIMNFFKTEITLANGEIRKRPLIETNGETGEIVW |
| | DKGRDFATVRKVLSMPQVNIVKKTEVQTGGFSKES |
| | ILPKRNSDKLIARKKDWDPKKYGGFDSPTVAYSVL |
| | VVAKVEKGKSKKLKSVKELLGITIMERSSFEKNPI |
| | DFLEAKGYKEVKKDLIIKLPKYSLFELENGRKRML |
| | ASAGELQKGNELALPSKYVNFLYLASHYEKLKGSP |
| | EDNEQKQLFVEQHKHYLDEIIEQISEFSKRVILAD |
| | ANLDKVLSAYNKHRDKPIREQAENIIHLFTLTNLG |
| | APAAFKYFDTTIDRKRYTSTKEVLDATLIHQSITG |
| | LYETRIDLSQLGGDSGGSSGGSSGSETPGTSESAT |
| | PESSGGSSGGSSTLNIEDEYRLHETSKEPDVSLGS |
| | TWLSDFPQAWAETGGMGLAVRQAPLIIPLKATSTP |
| | VSIKQYPMSQEARLGIKPHIQRLLDQGILVPCQSP |
| | WNTPLLPVKKPGTNDYRPVQDLREVNKRVEDIHPT |
| | VPNPYNLLSGLPPSHQWYTVLDLKDAFFCLRLHPT |
| | SQPLFAFEWRDPEMGISGQLTWTRLPQGFKNSPTL |
| | FNEALHRDLADFRIQHPDLILLQYVDDLLLAATSE |
| | LDCQQGTRALLQTLGNLGYRASAKKAQICQKQVKY |
| | LGYLLKEGQRWLTEARKETVMGQPTPKTPRQLREF |
| | LGKAGFCRLFIPGFAEMAAPLYPLTKPGTLFNWGP |
| | DQQKAYQEIKQALLTAPALGLPDLTKPFELFVDEK |
| | QGYAKGVLTQKLGPWRRPVAYLSKKLDPVAAGWPP |
| | CLRMVAAIAVLTKDAGKLTMGQPLVILAPHAVEAL |
| | VKQPPDRWLSNARMTHYQALLLDTDRVQFGPVVAL |
| | NPATLLPLPEEGLQHNCLDILAEAHGTRPDLTDQP |
| | LPDADHTWYTDGSSLLQEGQRKAGAAVTTETEVIW |
| | AKALPAGTSAQRAELIALTQALKMAEGKKLNVYTD |
| | SRYAFATAHIHGEIYRRRGWLTSEGKEIKNKDEIL |
| | ALLKALFLPKRLSIIHCPGHQKGHSAEARGNRMAD |
| | QAARKAAITETPDTSTLLIENSSPSGGSKRTADGS |
| | EFEPKKKRKV |
| iMM-N1054-PE | MKRTADGSEFESPKKKRKVDKKYSIGLDIGTNSVG |
| (SEQ ID | WAVITDEYKVPSKKFKVLGNTDRHSIKKNLIGALL |
| NO: 45) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ELDINRLSDYDVDAIVPQSFLKDDSIDNKVLTRSD |
| | KNRGKSDNVPSEEVVKKMKNYWRQLLNAKLITQRK |
| | FDNLTKAERGGLSELDKAGFIKRQLVETRQITKHV |
| | AQILDSRMNTKYDENDKLIREVKVITLKSKLVSDF |
| | RKDFQFYKVREINNYHHAHDAYLNAVVGTALIKKY |
| | PKLESEFVYGDYKVYDVRKMIAKSEQEIGKATAKY |
| | FFYSNIMNFFKTEITLANSGGSSGGSSGGSSGGSA |
| | SNFTQFVLVDNGGTGDVTVAPSNFANGVAEWISSN |
| | SRSQAYKVTCSVRQSSAQKRKYTIKVEVPKVATQT |
| | VGGVELPVAAWRSYLNMELTIPIFATNSDCELIVK |
| | AMQGLLKDGNPIPSAIAANSGIYGSSGSETPGTSE |
| | SATPESSGASNFTQFVLVDNGGTGDVTVAPSNFAN |
| | GVAEWISSNSRSQAYKVTCSVRQSSAQKRKYTIKV |
| | EVPKVATQTVGGVELPVAAWRSYLNMELTIPIFAT |
| | NSDCELIVKAMQGLLKDGNPIPSAIAANSGIYSAG |
| | GGGSGGGGSGGGGSGGEIRKRPLIETNGETGEIVW |
| | DKGRDFATVRKVLSMPQVNIVKKTEVQTGGFSKES |
| | ILPKRNSDKLIARKKDWDPKKYGGFDSPTVAYSVL |
| | VVAKVEKGKSKKLKSVKELLGITIMERSSFEKNPI |
| | DFLEAKGYKEVKKDLIIKLPKYSLFELENGRKRML |
| | ASAGELQKGNELALPSKYVNFLYLASHYEKLKGSP |
| | EDNEQKQLFVEQHKHYLDEIIEQISEFSKRVILAD |
| | ANLDKVLSAYNKHRDKPIREQAENIIHLFTLTNLG |
| | APAAFKYFDTTIDRKRYTSTKEVLDATLIHQSITG |
| | LYETRIDLSQLGGDSGGSSGGSSGSETPGTSESAT |
| | PESSGGSSGGSSTLNIEDEYRLHETSKEPDVSLGS |
| | TWLSDFPQAWAETGGMGLAVRQAPLIIPLKATSTP |
| | VSIKQYPMSQEARLGIKPHIQRLLDQGILVPCQSP |
| | WNTPLLPVKKPGTNDYRPVQDLREVNKRVEDIHPT |
| | VPNPYNLLSGLPPSHQWYTVLDLKDAFFCLRLHPT |
| | SQPLFAFEWRDPEMGISGQLTWTRLPQGFKNSPTL |
| | FNEALHRDLADFRIQHPDLILLQYVDDLLLAATSE |
| | LDCQQGTRALLQTLGNLGYRASAKKAQICQKQVKY |
| | LGYLLKEGQRWLTEARKETVMGQPTPKTPRQLREF |
| | LGKAGFCRLFIPGFAEMAAPLYPLTKPGTLFNWGP |
| | DQQKAYQEIKQALLTAPALGLPDLTKPFELFVDEK |
| | QGYAKGVLTQKLGPWRRPVAYLSKKLDPVAAGWPP |
| | CLRMVAAIAVLTKDAGKLTMGQPLVILAPHAVEAL |
| | VKQPPDRWLSNARMTHYQALLLDTDRVQFGPVVAL |
| | NPATLLPLPEEGLQHNCLDILAEAHGTRPDLTDQP |
| | LPDADHTWYTDGSSLLQEGQRKAGAAVTTETEVIW |
| | AKALPAGTSAQRAELIALTQALKMAEGKKLNVYTD |
| | SRYAFATAHIHGEIYRRRGWLTSEGKEIKNKDEIL |
| | ALLKALFLPKRLSIIHCPGHQKGHSAEARGNRMAD |
| | QAARKAAITETPDTSTLLIENSSPSGGSKRTADGS |
| | EFEPKKKRKV |
| iMM-G1247-PE | MKRTADGSEFESPKKKRKVDKKYSIGLDIGTNSVG |
| (SEQ ID NO: | WAVITDEYKVPSKKFKVLGNTDRHSIKKNLIGALL |
| 46) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ELDINRLSDYDVDAIVPQSFLKDDSIDNKVLTRSD |
| | KNRGKSDNVPSEEVVKKMKNYWRQLLNAKLITQRK |
| | FDNLTKAERGGLSELDKAGFIKRQLVETRQITKHV |
| | AQILDSRMNTKYDENDKLIREVKVITLKSKLVSDF |
| | RKDFQFYKVREINNYHHAHDAYLNAVVGTALIKKY |
| | PKLESEFVYGDYKVYDVRKMIAKSEQEIGKATAKY |
| | FFYSNIMNFFKTEITLANGEIRKRPLIETNGETGE |
| | IVWDKGRDFATVRKVLSMPQVNIVKKTEVQTGGFS |
| | KESILPKRNSDKLIARKKDWDPKKYGGFDSPTVAY |
| | SVLVVAKVEKGKSKKLKSVKELLGITIMERSSFEK |
| | NPIDFLEAKGYKEVKKDLIIKLPKYSLFELENGRK |
| | RMLASAGELQKGNELALPSKYVNFLYLASHYEKLK |
| | GSGGSSGGSSGGSSGGSASNFTQFVLVDNGGTGDV |
| | TVAPSNFANGVAEWISSNSRSQAYKVTCSVRQSSA |
| | QKRKYTIKVEVPKVATQTVGGVELPVAAWRSYLNM |
| | ELTIPIFATNSDCELIVKAMQGLLKDGNPIPSAIA |
| | ANSGIYGSSGSETPGTSESATPESSGASNFTQFVL |
| | VDNGGTGDVTVAPSNFANGVAEWISSNSRSQAYKV |
| | TCSVRQSSAQKRKYTIKVEVPKVATQTVGGVELPV |
| | AAWRSYLNMELTIPIFATNSDCELIVKAMQGLLKD |
| | GNPIPSAIAANSGIYSAGGGGSGGGGSGGGGSGSP |
| | EDNEQKQLFVEQHKHYLDEIIEQISEFSKRVILAD |
| | ANLDKVLSAYNKHRDKPIREQAENIIHLFTLTNLG |
| | APAAFKYFDTTIDRKRYTSTKEVLDATLIHQSITG |
| | LYETRIDLSQLGGDSGGSSGGSSGSETPGTSESAT |
| | PESSGGSSGGSSTLNIEDEYRLHETSKEPDVSLGS |
| | TWLSDFPQAWAETGGMGLAVRQAPLIIPLKATSTP |
| | VSIKQYPMSQEARLGIKPHIQRLLDQGILVPCQSP |
| | WNTPLLPVKKPGTNDYRPVQDLREVNKRVEDIHPT |
| | VPNPYNLLSGLPPSHQWYTVLDLKDAFFCLRLHPT |
| | SQPLFAFEWRDPEMGISGQLTWTRLPQGFKNSPTL |
| | FNEALHRDLADFRIQHPDLILLQYVDDLLLAATSE |
| | LDCQQGTRALLQTLGNLGYRASAKKAQICQKQVKY |
| | LGYLLKEGQRWLTEARKETVMGQPTPKTPRQLREF |
| | LGKAGFCRLFIPGFAEMAAPLYPLTKPGTLFNWGP |
| | DQQKAYQEIKQALLTAPALGLPDLTKPFELFVDEK |
| | QGYAKGVLTQKLGPWRRPVAYLSKKLDPVAAGWPP |
| | CLRMVAAIAVLTKDAGKLTMGQPLVILAPHAVEAL |
| | VKQPPDRWLSNARMTHYQALLLDTDRVQFGPVVAL |
| | NPATLLPLPEEGLQHNCLDILAEAHGTRPDLTDQP |
| | LPDADHTWYTDGSSLLQEGQRKAGAAVTTETEVIW |
| | AKALPAGTSAQRAELIALTQALKMAEGKKLNVYTD |
| | SRYAFATAHIHGEIYRRRGWLTSEGKEIKNKDEIL |
| | ALLKALFLPKRLSIIHCPGHQKGHSAEARGNRMAD |
| | QAARKAAITETPDTSTLLIENSSPSGGSKRTADGS |
| | EFEPKKKRKV |
| iMM-D1299-PE | MKRTADGSEFESPKKKRKVDKKYSIGLDIGTNSVG |
| (SEQ ID NO: | WAVITDEYKVPSKKFKVLGNTDRHSIKKNLIGALL |
| 47) | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ELDINRLSDYDVDAIVPQSFLKDDSIDNKVLTRSD |
| | KNRGKSDNVPSEEVVKKMKNYWRQLLNAKLITQRK |
| | FDNLTKAERGGLSELDKAGFIKRQLVETRQITKHV |
| | AQILDSRMNTKYDENDKLIREVKVITLKSKLVSDF |
| | RKDFQFYKVREINNYHHAHDAYLNAVVGTALIKKY |
| | PKLESEFVYGDYKVYDVRKMIAKSEQEIGKATAKY |
| | FFYSNIMNFFKTEITLANGEIRKRPLIETNGETGE |
| | IVWDKGRDFATVRKVLSMPQVNIVKKTEVQTGGFS |
| | KESILPKRNSDKLIARKKDWDPKKYGGFDSPTVAY |
| | SVLVVAKVEKGKSKKLKSVKELLGITIMERSSFEK |
| | NPIDFLEAKGYKEVKKDLIIKLPKYSLFELENGRK |
| | RMLASAGELQKGNELALPSKYVNFLYLASHYEKLK |
| | GSPEDNEQKQLFVEQHKHYLDEIIEQISEFSKRVI |
| | LADANLDKVLSAYNKHRDSGGSSGGSSGGSSGGSA |
| | SNFTQFVLVDNGGTGDVTVAPSNFANGVAEWISSN |
| | SRSQAYKVTCSVRQSSAQKRKYTIKVEVPKVATQT |
| | VGGVELPVAAWRSYLNMELTIPIFATNSDCELIVK |
| | AMQGLLKDGNPIPSAIAANSGIYGSSGSETPGTSE |
| | SATPESSGASNFTQFVLVDNGGTGDVTVAPSNFAN |
| | GVAEWISSNSRSQAYKVTCSVRQSSAQKRKYTIKV |
| | EVPKVATQTVGGVELPVAAWRSYLNMELTIPIFAT |
| | NSDCELIVKAMQGLLKDGNPIPSAIAANSGIYSAG |
| | GGGSGGGGSGGGGSGKPIREQAENIIHLFTLTNLG |
| | APAAFKYFDTTIDRKRYTSTKEVLDATLIHQSITG |
| | LYETRIDLSQLGGDSGGSSGGSSGSETPGTSESAT |
| | PESSGGSSGGSSTLNIEDEYRLHETSKEPDVSLGS |
| | TWLSDFPQAWAETGGMGLAVRQAPLIIPLKATSTP |
| | VSIKQYPMSQEARLGIKPHIQRLLDQGILVPCQSP |
| | WNTPLLPVKKPGTNDYRPVQDLREVNKRVEDIHPT |
| | VPNPYNLLSGLPPSHQWYTVLDLKDAFFCLRLHPT |
| | SQPLFAFEWRDPEMGISGQLTWTRLPQGFKNSPTL |
| | FNEALHRDLADFRIQHPDLILLQYVDDLLLAATSE |
| | LDCQQGTRALLQTLGNLGYRASAKKAQICQKQVKY |
| | LGYLLKEGQRWLTEARKETVMGQPTPKTPRQLREF |
| | LGKAGFCRLFIPGFAEMAAPLYPLTKPGTLFNWGP |
| | DQQKAYQEIKQALLTAPALGLPDLTKPFELFVDEK |
| | QGYAKGVLTQKLGPWRRPVAYLSKKLDPVAAGWPP |
| | CLRMVAAIAVLTKDAGKLTMGQPLVILAPHAVEAL |
| | VKQPPDRWLSNARMTHYQALLLDTDRVQFGPVVAL |
| | NPATLLPLPEEGLQHNCLDILAEAHGTRPDLTDQP |
| | LPDADHTWYTDGSSLLQEGQRKAGAAVTTETEVIW |
| | AKALPAGTSAQRAELIALTQALKMAEGKKLNVYTD |
| | SRYAFATAHIHGEIYRRRGWLTSEGKEIKNKDEIL |
| | ALLKALFLPKRLSIIHCPGHQKGHSAEARGNRMAD |
| | QAARKAAITETPDTSTLLIENSSPSGGSKRTADGS |
| | EFEPKKKRKV |
| iMM-E827-PE | MKRTADGSEFESPKKKRKVDKKYSIGLDIGTNSVG |
| (SEQ ID NO: 48) | WAVITDEYKVPSKKFKVLGNTDRHSIKKNLIGALL |
| | FDSGETAEATRLKRTARRRYTRRKNRICYLQEIFS |
| | NEMAKVDDSFFHRLEESFLVEEDKKHERHPIFGNI |
| | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSQILKEHPVENTQLQNEKLYLLQNGRDMYVDQ |
| | ESGGSSGGSSGGSSGGSASNFTQFVLVDNGGTGDV |
| | TVAPSNFANGVAEWISSNSRSQAYKVTCSVRQSSA |
| | QKRKYTIKVEVPKVATQTVGGVELPVAAWRSYLNM |
| | ELTIPIFATNSDCELIVKAMQGLLKDGNPIPSAIA |
| | ANSGIYGSSGSETPGTSESATPESSGASNFTQFVL |
| | VDNGGTGDVTVAPSNFANGVAEWISSNSRSQAYKV |
| | TCSVRQSSAQKRKYTIKVEVPKVATQTVGGVELPV |
| | AAWRSYLNMELTIPIFATNSDCELIVKAMQGLLKD |
| | GNPIPSAIAANSGIYSAGGGGSGGGGSGGGGSGLD |
| | INRLSDYDVDAIVPQSFLKDDSIDNKVLTRSDKNR |
| | GKSDNVPSEEVVKKMKNYWRQLLNAKLITQRKFDN |
| | LTKAERGGLSELDKAGFIKRQLVETRQITKHVAQI |
| | LDSRMNTKYDENDKLIREVKVITLKSKLVSDFRKD |
| | FQFYKVREINNYHHAHDAYLNAVVGTALIKKYPKL |
| | ESEFVYGDYKVYDVRKMIAKSEQEIGKATAKYFFY |
| | SNIMNFFKTEITLANGEIRKRPLIETNGETGEIVW |
| | DKGRDFATVRKVLSMPQVNIVKKTEVQTGGFSKES |
| | ILPKRNSDKLIARKKDWDPKKYGGFDSPTVAYSVL |
| | VVAKVEKGKSKKLKSVKELLGITIMERSSFEKNPI |
| | DFLEAKGYKEVKKDLIIKLPKYSLFELENGRKRML |
| | ASAGELQKGNELALPSKYVNFLYLASHYEKLKGSP |
| | EDNEQKQLFVEQHKHYLDEIIEQISEFSKRVILAD |
| | ANLDKVLSAYNKHRDKPIREQAENIIHLFTLTNLG |
| | APAAFKYFDTTIDRKRYTSTKEVLDATLIHQSITG |
| | LYETRIDLSQLGGDSGGSSGGSSGSETPGTSESAT |
| | PESSGGSSGGSSTLNIEDEYRLHETSKEPDVSLGS |
| | TWLSDFPQAWAETGGMGLAVRQAPLIIPLKATSTP |
| | VSIKQYPMSQEARLGIKPHIQRLLDQGILVPCQSP |
| | WNTPLLPVKKPGTNDYRPVQDLREVNKRVEDIHPT |
| | VPNPYNLLSGLPPSHQWYTVLDLKDAFFCLRLHPT |
| | SQPLFAFEWRDPEMGISGQLTWTRLPQGFKNSPTL |
| | FNEALHRDLADFRIQHPDLILLQYVDDLLLAATSE |
| | LDCQQGTRALLQTLGNLGYRASAKKAQICQKQVKY |
| | LGYLLKEGQRWLTEARKETVMGQPTPKTPRQLREF |
| | LGKAGFCRLFIPGFAEMAAPLYPLTKPGTLFNWGP |
| | DQQKAYQEIKQALLTAPALGLPDLTKPFELFVDEK |
| | QGYAKGVLTQKLGPWRRPVAYLSKKLDPVAAGWPP |
| | CLRMVAAIAVLTKDAGKLTMGQPLVILAPHAVEAL |
| | VKQPPDRWLSNARMTHYQALLLDTDRVQFGPVVAL |
| | NPATLLPLPEEGLQHNCLDILAEAHGTRPDLTDQP |
| | LPDADHTWYTDGSSLLQEGQRKAGAAVTTETEVIW |
| | AKALPAGTSAQRAELIALTQALKMAEGKKLNVYTD |
| | SRYAFATAHIHGEIYRRRGWLTSEGKEIKNKDEIL |
| | ALLKALFLPKRLSIIHCPGHQKGHSAEARGNRMAD |
| | QAARKAAITETPDTSTLLIENSSPSGGSKRTADGS |
| | EFEPKKKRKV |
| IMM-delta | MKRTADGSEFESPKKKRKVDKKYSIGLDIGTNSVG |
| (S793- | WAVITDEYKVPSKKFKVLGNTDRHSIKKNLIGALL |
| R905)-PE | FDSGETAEATRLKRTARRRYTRRKNRICYLQEIFS |
| (SEQ ID NO: | NEMAKVDDSFFHRLEESFLVEEDKKHERHPIFGNI |
| 49) | VDEVAYHEKYPTIYHLRKKLVDSTDKADLRLIYLA |
| | LAHMIKFRGHFLIEGDLNPDNSDVDKLFIQLVQTY |
| | NQLFEENPINASGVDAKAILSARLSKSRRLENLIA |
| | QLPGEKKNGLFGNLIALSLGLTPNFKSNFDLAEDA |
| | KLQLSKDTYDDDLDNLLAQIGDQYADLFLAAKNLS |
| | DAILLSDILRVNTEITKAPLSASMIKRYDEHHQDL |
| | TLLKALVRQQLPEKYKEIFFDQSKNGYAGYIDGGA |
| | SQEEFYKFIKPILEKMDGTEELLVKLNREDLLRKQ |
| | RTFDNGSIPHQIHLGELHAILRRQEDFYPFLKDNR |
| | EKIEKILTFRIPVGPLARGNSRFAWMTRKSEETIT |
| | PWNFEEVVDKGASAQSFIERMTNFDKNLPNEKVLP |
| | KHSLLYEYFTVYNELTKVKYVTEGMRKPAFLSGEQ |
| | KKAIVDLLFKTNRKVTVKQLKEDYFKKIECFDSVE |
| | ISGVEDRFNASLGTYHDLLKIIKDKDFLDNEENED |
| | ILEDIVLTLTLFEDREMIEERLKTYAHLFDDKVMK |
| | QLKRRRYTGWGRLSRKLINGIRDKQSGKTILDFLK |
| | SDGFANRNFMQLIHDDSLTFKEDIQKAQVSGQGDS |
| | LHEHIANLAGSPAIKKGILQTVKVVDELVKVMGRH |
| | KPENIVIEMARENQTTQKGQKNSRERMKRIEEGIK |
| | ELGSGGSSGGSSGGSSGGSASNFTQFVLVDNGGTG |
| | DVTVAPSNFANGVAEWISSNSRSQAYKVTCSVRQS |
| | SAQKRKYTIKVEVPKVATQTVGGVELPVAAWRSYL |
| | NMELTIPIFATNSDCELIVKAMQGLLKDGNPIPSA |
| | IAANSGIYGSSGSETPGTSESATPESSGASNFTQF |
| | VLVDNGGTGDVTVAPSNFANGVAEWISSNSRSQAY |
| | KVTCSVRQSSAQKRKYTIKVEVPKVATQTVGGVEL |
| | PVAAWRSYLNMELTIPIFATNSDCELIVKAMQGLL |
| | KDGNPIPSAIAANSGIYSAGGGGSGGGGSGGGGSG |
| | GGLSELDKAGFIKRQLVETRQITKHVAQILDSRMN |
| | TKYDENDKLIREVKVITLKSKLVSDFRKDFQFYKV |
| | REINNYHHAHDAYLNAVVGTALIKKYPKLESEFVY |
| | GDYKVYDVRKMIAKSEQEIGKATAKYFFYSNIMNF |
| | FKTEITLANGEIRKRPLIETNGETGEIVWDKGRDF |
| | ATVRKVLSMPQVNIVKKTEVQTGGFSKESILPKRN |
| | SDKLIARKKDWDPKKYGGFDSPTVAYSVLVVAKVE |
| | KGKSKKLKSVKELLGITIMERSSFEKNPIDFLEAK |
| | GYKEVKKDLIIKLPKYSLFELENGRKRMLASAGEL |
| | QKGNELALPSKYVNFLYLASHYEKLKGSPEDNEQK |
| | QLFVEQHKHYLDEIIEQISEFSKRVILADANLDKV |
| | LSAYNKHRDKPIREQAENIIHLFTLTNLGAPAAFK |
| | YFDTTIDRKRYTSTKEVLDATLIHQSITGLYETRI |
| | DLSQLGGDSGGSSGGSSGSETPGTSESATPESSGG |
| | SSGGSSTLNIEDEYRLHETSKEPDVSLGSTWLSDF |
| | PQAWAETGGMGLAVRQAPLIIPLKATSTPVSIKQY |
| | PMSQEARLGIKPHIQRLLDQGILVPCQSPWNTPLL |
| | PVKKPGTNDYRPVQDLREVNKRVEDIHPTVPNPYN |
| | LLSGLPPSHQWYTVLDLKDAFFCLRLHPTSQPLFA |
| | FEWRDPEMGISGQLTWTRLPQGFKNSPTLFNEALH |
| | RDLADFRIQHPDLILLQYVDDLLLAATSELDCQQG |
| | TRALLQTLGNLGYRASAKKAQICQKQVKYLGYLLK |
| | EGQRWLTEARKETVMGQPTPKTPRQLREFLGKAGF |
| | CRLFIPGFAEMAAPLYPLTKPGTLFNWGPDQQKAY |
| | QEIKQALLTAPALGLPDLTKPFELFVDEKQGYAKG |
| | VLTQKLGPWRRPVAYLSKKLDPVAAGWPPCLRMVA |
| | AIAVLTKDAGKLTMGQPLVILAPHAVEALVKQPPD |
| | RWLSNARMTHYQALLLDTDRVQFGPVVALNPATLL |
| | PLPEEGLQHNCLDILAEAHGTRPDLTDQPLPDADH |
| | TWYTDGSSLLQEGQRKAGAAVTTETEVIWAKALPA |
| | GTSAQRAELIALTQALKMAEGKKLNVYTDSRYAFA |
| | TAHIHGEIYRRRGWLTSEGKEIKNKDEILALLKAL |
| | FLPKRLSIIHCPGHQKGHSAEARGNRMADQAARKA |
| | AITETPDTSTLLIENSSPSGGSKRTADGSEFEPKK |
| | KRKV |
| nMMMM-PE | MKRTADGSEFESPKKKRKVASNFTQFVLVDNGGTG |
| (SEQ ID NO: | DVTVAPSNFANGVAEWISSNSRSQAYKVTCSVRQS |
| 50) | SAQKRKYTIKVEVPKVATQTVGGVELPVAAWRSYL |
| | NMELTIPIFATNSDCELIVKAMQGLLKDGNPIPSA |
| | IAANSGIYGSSGSETPGTSESATPESSGASNFTQF |
| | VLVDNGGTGDVTVAPSNFANGVAEWISSNSRSQAY |
| | KVTCSVRQSSAQKRKYTIKVEVPKVATQTVGGVEL |
| | PVAAWRSYLNMELTIPIFATNSDCELIVKAMQGLL |
| | KDGNPIPSAIAANSGIYSAGGGGSGGGGSGGGGSG |
| | ASNFTQFVLVDNGGTGDVTVAPSNFANGVAEWISS |
| | NSRSQAYKVTCSVRQSSAQKRKYTIKVEVPKVATQ |
| | TVGGVELPVAAWRSYLNMELTIPIFATNSDCELIV |
| | KAMQGLLKDGNPIPSAIAANSGIYGSSGSETPGTS |
| | ESATPESSGASNFTQFVLVDNGGTGDVTVAPSNFA |
| | NGVAEWISSNSRSQAYKVTCSVRQSSAQKRKYTIK |
| | VEVPKVATQTVGGVELPVAAWRSYLNMELTIPIFA |
| | TNSDCELIVKAMQGLLKDGNPIPSAIAANSGIYSA |
| | GGGGSGGGGSGGGGSGPKKKRKVAAAGSDKKYSIG |
| | LDIGTNSVGWAVITDEYKVPSKKFKVLGNTDRHSI |
| | KKNLIGALLFDSGETAEATRLKRTARRRYTRRKNR |
| | ICYLQEIFSNEMAKVDDSFFHRLEESFLVEEDKKH |
| | ERHPIFGNIVDEVAYHEKYPTIYHLRKKLVDSTDK |
| | ADLRLIYLALAHMIKFRGHFLIEGDLNPDNSDVDK |
| | LFIQLVQTYNQLFEENPINASGVDAKAILSARLSK |
| | SRRLENLIAQLPGEKKNGLFGNLIALSLGLTPNFK |
| | SNFDLAEDAKLQLSKDTYDDDLDNLLAQIGDQYAD |
| | LFLAAKNLSDAILLSDILRVNTEITKAPLSASMIK |
| | RYDEHHQDLTLLKALVRQQLPEKYKEIFFDQSKNG |
| | YAGYIDGGASQEEFYKFIKPILEKMDGTEELLVKL |
| | NREDLLRKQRTFDNGSIPHQIHLGELHAILRRQED |
| | FYPFLKDNREKIEKILTFRIPVGPLARGNSRFAWM |
| | TRKSEETITPWNFEEVVDKGASAQSFIERMTNFDK |
| | NLPNEKVLPKHSLLYEYFTVYNELTKVKYVTEGMR |
| | KPAFLSGEQKKAIVDLLFKTNRKVTVKQLKEDYFK |
| | KIECFDSVEISGVEDRFNASLGTYHDLLKIIKDKD |
| | FLDNEENEDILEDIVLTLTLFEDREMIEERLKTYA |
| | HLFDDKVMKQLKRRRYTGWGRLSRKLINGIRDKQS |
| | GKTILDFLKSDGFANRNFMQLIHDDSLTFKEDIQK |
| | AQVSGQGDSLHEHIANLAGSPAIKKGILQTVKVVD |
| | ELVKVMGRHKPENIVIEMARENQTTQKGQKNSRER |
| | MKRIEEGIKELGSQILKEHPVENTQLQNEKLYLLQ |
| | NGRDMYVDQELDINRLSDYDVDAIVPQSFLKDDSI |
| | DNKVLTRSDKNRGKSDNVPSEEVVKKMKNYWRQLL |
| | NAKLITQRKFDNLTKAERGGLSELDKAGFIKRQLV |
| | ETRQITKHVAQILDSRMNTKYDENDKLIREVKVIT |
| | LKSKLVSDFRKDFQFYKVREINNYHHAHDAYLNAV |
| | VGTALIKKYPKLESEFVYGDYKVYDVRKMIAKSEQ |
| | EIGKATAKYFFYSNIMNFFKTEITLANGEIRKRPL |
| | IETNGETGEIVWDKGRDFATVRKVLSMPQVNIVKK |
| | TEVQTGGFSKESILPKRNSDKLIARKKDWDPKKYG |
| | GFDSPTVAYSVLVVAKVEKGKSKKLKSVKELLGIT |
| | IMERSSFEKNPIDFLEAKGYKEVKKDLIIKLPKYS |
| | LFELENGRKRMLASAGELQKGNELALPSKYVNFLY |
| | LASHYEKLKGSPEDNEQKQLFVEQHKHYLDEIIEQ |
| | ISEFSKRVILADANLDKVLSAYNKHRDKPIREQAE |
| | NIIHLFTLTNLGAPAAFKYFDTTIDRKRYTSTKEV |
| | LDATLIHQSITGLYETRIDLSQLGGDSGGSSGGSS |
| | GSETPGTSESATPESSGGSSGGSSTLNIEDEYRLH |
| | ETSKEPDVSLGSTWLSDFPQAWAETGGMGLAVRQA |
| | PLIIPLKATSTPVSIKQYPMSQEARLGIKPHIQRL |
| | LDQGILVPCQSPWNTPLLPVKKPGTNDYRPVQDLR |
| | EVNKRVEDIHPTVPNPYNLLSGLPPSHQWYTVLDL |
| | KDAFFCLRLHPTSQPLFAFEWRDPEMGISGQLTWT |
| | RLPQGFKNSPTLFNEALHRDLADFRIQHPDLILLQ |
| | YVDDLLLAATSELDCQQGTRALLQTLGNLGYRASA |
| | KKAQICQKQVKYLGYLLKEGQRWLTEARKETVMGQ |
| | PTPKTPRQLREFLGKAGFCRLFIPGFAEMAAPLYP |
| | LTKPGTLFNWGPDQQKAYQEIKQALLTAPALGLPD |
| | LTKPFELFVDEKQGYAKGVLTQKLGPWRRPVAYLS |
| | KKLDPVAAGWPPCLRMVAAIAVLTKDAGKLTMGQP |
| | LVILAPHAVEALVKQPPDRWLSNARMTHYQALLLD |
| | TDRVQFGPVVALNPATLLPLPEEGLQHNCLDILAE |
| | AHGTRPDLTDQPLPDADHTWYTDGSSLLQEGQRKA |
| | GAAVTTETEVIWAKALPAGTSAQRAELIALTQALK |
| | MAEGKKLNVYTDSRYAFATAHIHGEIYRRRGWLTS |
| | EGKEIKNKDEILALLKALFLPKRLSIIHCPGHQKG |
| | HSAEARGNRMADQAARKAAITETPDTSTLLIENSS |
| | PSGGSKRTADGSEFEPKKKRKV |
| nMMcMM-PE | MKRTADGSEFESPKKKRKVASNFTQFVLVDNGGTG |
| (SEQ ID NO: | DVTVAPSNFANGVAEWISSNSRSQAYKVTCSVRQS |
| 51) | SAQKRKYTIKVEVPKVATQTVGGVELPVAAWRSYL |
| | NMELTIPIFATNSDCELIVKAMQGLLKDGNPIPSA |
| | IAANSGIYGSSGSETPGTSESATPESSGASNFTQF |
| | VLVDNGGTGDVTVAPSNFANGVAEWISSNSRSQAY |
| | KVTCSVRQSSAQKRKYTIKVEVPKVATQTVGGVEL |
| | PVAAWRSYLNMELTIPIFATNSDCELIVKAMQGLL |
| | KDGNPIPSAIAANSGIYSAGGGGSGGGGSGGGGSG |
| | PKKKRKVAAAGSDKKYSIGLDIGTNSVGWAVITDE |
| | YKVPSKKFKVLGNTDRHSIKKNLIGALLFDSGETA |
| | EATRLKRTARRRYTRRKNRICYLQEIFSNEMAKVD |
| | DSFFHRLEESFLVEEDKKHERHPIFGNIVDEVAYH |
| | EKYPTIYHLRKKLVDSTDKADLRLIYLALAHMIKF |
| | RGHFLIEGDLNPDNSDVDKLFIQLVQTYNQLFEEN |
| | PINASGVDAKAILSARLSKSRRLENLIAQLPGEKK |
| | NGLFGNLIALSLGLTPNFKSNFDLAEDAKLQLSKD |
| | TYDDDLDNLLAQIGDQYADLFLAAKNLSDAILLSD |
| | ILRVNTEITKAPLSASMIKRYDEHHQDLTLLKALV |
| | RQQLPEKYKEIFFDQSKNGYAGYIDGGASQEEFYK |
| | FIKPILEKMDGTEELLVKLNREDLLRKQRTFDNGS |
| | IPHQIHLGELHAILRRQEDFYPFLKDNREKIEKIL |
| | TFRIPVGPLARGNSRFAWMTRKSEETITPWNFEEV |
| | VDKGASAQSFIERMTNFDKNLPNEKVLPKHSLLYE |
| | YFTVYNELTKVKYVTEGMRKPAFLSGEQKKAIVDL |
| | LFKTNRKVTVKQLKEDYFKKIECFDSVEISGVEDR |
| | FNASLGTYHDLLKIIKDKDFLDNEENEDILEDIVL |
| | TLTLFEDREMIEERLKTYAHLFDDKVMKQLKRRRY |
| | TGWGRLSRKLINGIRDKQSGKTILDFLKSDGFANR |
| | NFMQLIHDDSLTFKEDIQKAQVSGQGDSLHEHIAN |
| | LAGSPAIKKGILQTVKVVDELVKVMGRHKPENIVI |
| | EMARENQTTQKGQKNSRERMKRIEEGIKELGSQIL |
| | KEHPVENTQLQNEKLYLLQNGRDMYVDQELDINRL |
| | SDYDVDAIVPQSFLKDDSIDNKVLTRSDKNRGKSD |
| | NVPSEEVVKKMKNYWRQLLNAKLITQRKFDNLTKA |
| | ERGGLSELDKAGFIKRQLVETRQITKHVAQILDSR |
| | MNTKYDENDKLIREVKVITLKSKLVSDFRKDFQFY |
| | KVREINNYHHAHDAYLNAVVGTALIKKYPKLESEF |
| | VYGDYKVYDVRKMIAKSEQEIGKATAKYFFYSNIM |
| | NFFKTEITLANGEIRKRPLIETNGETGEIVWDKGR |
| | DFATVRKVLSMPQVNIVKKTEVQTGGFSKESILPK |
| | RNSDKLIARKKDWDPKKYGGFDSPTVAYSVLVVAK |
| | VEKGKSKKLKSVKELLGITIMERSSFEKNPIDFLE |
| | AKGYKEVKKDLIIKLPKYSLFELENGRKRMLASAG |
| | ELQKGNELALPSKYVNFLYLASHYEKLKGSPEDNE |
| | QKQLFVEQHKHYLDEIIEQISEFSKRVILADANLD |
| | KVLSAYNKHRDKPIREQAENIIHLFTLTNLGAPAA |
| | FKYFDTTIDRKRYTSTKEVLDATLIHQSITGLYET |
| | RIDLSQLGGDSGGSSGGSSGSETPGTSESATPESS |
| | GGSSGGSSTLNIEDEYRLHETSKEPDVSLGSTWLS |
| | DFPQAWAETGGMGLAVRQAPLIIPLKATSTPVSIK |
| | QYPMSQEARLGIKPHIQRLLDQGILVPCQSPWNTP |
| | LLPVKKPGTNDYRPVQDLREVNKRVEDIHPTVPNP |
| | YNLLSGLPPSHQWYTVLDLKDAFFCLRLHPTSQPL |
| | FAFEWRDPEMGISGQLTWTRLPQGFKNSPTLFNEA |
| | LHRDLADFRIQHPDLILLQYVDDLLLAATSELDCQ |
| | QGTRALLQTLGNLGYRASAKKAQICQKQVKYLGYL |
| | LKEGQRWLTEARKETVMGQPTPKTPRQLREFLGKA |
| | GFCRLFIPGFAEMAAPLYPLTKPGTLFNWGPDQQK |
| | AYQEIKQALLTAPALGLPDLTKPFELFVDEKQGYA |
| | KGVLTQKLGPWRRPVAYLSKKLDPVAAGWPPCLRM |
| | VAAIAVLTKDAGKLTMGQPLVILAPHAVEALVKQP |
| | PDRWLSNARMTHYQALLLDTDRVQFGPVVALNPAT |
| | LLPLPEEGLQHNCLDILAEAHGTRPDLTDQPLPDA |
| | DHTWYTDGSSLLQEGQRKAGAAVTTETEVIWAKAL |
| | PAGTSAQRAELIALTQALKMAEGKKLNVYTDSRYA |
| | FATAHIHGEIYRRRGWLTSEGKEIKNKDEILALLK |
| | ALFLPKRLSIIHCPGHQKGHSAEARGNRMADQAAR |
| | KAAITETPDTSTLLIENSSPSGGSSGGSSGGSSGG |
| | SASNFTQFVLVDNGGTGDVTVAPSNFANGVAEWIS |
| | SNSRSQAYKVTCSVRQSSAQKRKYTIKVEVPKVAT |
| | QTVGGVELPVAAWRSYLNMELTIPIFATNSDCELI |
| | VKAMQGLLKDGNPIPSAIAANSGIYGSSGSETPGT |
| | SESATPESSGASNFTQFVLVDNGGTGDVTVAPSNF |
| | ANGVAEWISSNSRSQAYKVTCSVRQSSAQKRKYTI |
| | KVEVPKVATQTVGGVELPVAAWRSYLNMELTIPIF |
| | ATNSDCELIVKAMQGLLKDGNPIPSAIAANSGIYS |
| | GGSKRTADGSEFEPKKKRKV |
| nMM-iMM- | MKRTADGSEFESPKKKRKVASNFTQFVLVDNGGTG |
| G1247-PE | DVTVAPSNFANGVAEWISSNSRSQAYKVTCSVRQS |
| (SEQ ID NO: | SAQKRKYTIKVEVPKVATQTVGGVELPVAAWRSYL |
| 52) | NMELTIPIFATNSDCELIVKAMQGLLKDGNPIPSA |
| | IAANSGIYGSSGSETPGTSESATPESSGASNFTQF |
| | VLVDNGGTGDVTVAPSNFANGVAEWISSNSRSQAY |
| | KVTCSVRQSSAQKRKYTIKVEVPKVATQTVGGVEL |
| | PVAAWRSYLNMELTIPIFATNSDCELIVKAMQGLL |
| | KDGNPIPSAIAANSGIYSAGGGGSG |
| | GGGSGGGGSGPKKKRKVAAAGSDKKYSIGLDIGTN |
| | SVGWAVITDEYKVPSKKFKVLGNTDRHSIKKNLIG |
| | ALLFDSGETAEATRLKRTARRRYTRRKNRICYLQE |
| | IFSNEMAKVDDSFFHRLEESFLVEEDKKHERHPIF |
| | GNIVDEVAYHEKYPTIYHLRKKLVDSTDKADLRLI |
| | YLALAHMIKFRGHFLIEGDLNPDNSDVDKLFIQLV |
| | QTYNQLFEENPINASGVDAKAILSARLSKSRRLEN |
| | LIAQLPGEKKNGLFGNLIALSLGLTPNFKSNFDLA |
| | EDAKLQLSKDTYDDDLDNLLAQIGDQYADLFLAAK |
| | NLSDAILLSDILRVNTEITKAPLSASMIKRYDEHH |
| | QDLTLLKALVRQQLPEKYKEIFFDQSKNGYAGYID |
| | GGASQEEFYKFIKPILEKMDGTEELLVKLNREDLL |
| | RKQRTFDNGSIPHQIHLGELHAILRRQEDFYPFLK |
| | DNREKIEKILTFRIPVGPLARGNSRFAWMTRKSEE |
| | TITPWNFEEVVDKGASAQSFIERMTNFDKNLPNEK |
| | VLPKHSLLYEYFTVYNELTKVKYVTEGMRKPAFLS |
| | GEQKKAIVDLLFKTNRKVTVKQLKEDYFKKIECFD |
| | SVEISGVEDRFNASLGTYHDLLKIIKDKDFLDNEE |
| | NEDILEDIVLTLTLFEDREMIEERLKTYAHLFDDK |
| | VMKQLKRRRYTGWGRLSRKLINGIRDKQSGKTILD |
| | FLKSDGFANRNFMQLIHDDSLTFKEDIQKAQVSGQ |
| | GDSLHEHIANLAGSPAIKKGILQTVKVVDELVKVM |
| | GRHKPENIVIEMARENQTTQKGQKNSRERMKRIEE |
| | GIKELGSQILKEHPVENTQLQNEKLYLLQNGRDMY |
| | VDQELDINRLSDYDVDAIVPQSFLKDDSIDNKVLT |
| | RSDKNRGKSDNVPSEEVVKKMKNYWRQLLNAKLIT |
| | QRKFDNLTKAERGGLSELDKAGFIKRQLVETRQIT |
| | KHVAQILDSRMNTKYDENDKLIREVKVITLKSKLV |
| | SDFRKDFQFYKVREINNYHHAHDAYLNAVVGTALI |
| | KKYPKLESEFVYGDYKVYDVRKMIAKSEQEIGKAT |
| | AKYFFYSNIMNFFKTEITLANGEIRKRPLIETNGE |
| | TGEIVWDKGRDFATVRKVLSMPQVNIVKKTEVQTG |
| | GFSKESILPKRNSDKLIARKKDWDPKKYGGFDSPT |
| | VAYSVLVVAKVEKGKSKKLKSVKELLGITIMERSS |
| | FEKNPIDFLEAKGYKEVKKDLIIKLPKYSLFELEN |
| | GRKRMLASAGELQKGNELALPSKYVNFLYLASHYE |
| | KLKGSGGSSGGSSGGSSGGSASNFTQFVLVDNGGT |
| | GDVTVAPSNFANGVAEWISSNSRSQAYKVTCSVRQ |
| | SSAQKRKYTIKVEVPKVATQTVGGVELPVAAWRSY |
| | LNMELTIPIFATNSDCELIVKAMQGLLKDGNPIPS |
| | AIAANSGIYGSSGSETPGTSESATPESSGASNFTQ |
| | FVLVDNGGTGDVTVAPSNFANGVAEWISSNSRSQA |
| | YKVTCSVRQSSAQKRKYTIKVEVPKVATQTVGGVE |
| | LPVAAWRSYLNMELTIPIFATNSDCELIVKAMQGL |
| | LKDGNPIPSAIAANSGIYSAGGGGSGGGGSGGGGS |
| | GSPEDNEQKQLFVEQHKHYLDEIIEQISEFSKRVI |
| | LADANLDKVLSAYNKHRDKPIREQAENIIHLFTLT |
| | NLGAPAAFKYFDTTIDRKRYTSTKEVLDATLIHQS |
| | ITGLYETRIDLSQLGGDSGGSSGGSSGSETPGTSE |
| | SATPESSGGSSGGSSTLNIEDEYRLHETSKEPDVS |
| | LGSTWLSDFPQAWAETGGMGLAVRQAPLIIPLKAT |
| | STPVSIKQYPMSQEARLGIKPHIQRLLDQGILVPC |
| | QSPWNTPLLPVKKPGTNDYRPVQDLREVNKRVEDI |
| | HPTVPNPYNLLSGLPPSHQWYTVLDLKDAFFCLRL |
| | HPTSQPLFAFEWRDPEMGISGQLTWTRLPQGFKNS |
| | PTLFNEALHRDLADFRIQHPDLILLQYVDDLLLAA |
| | TSELDCQQGTRALLQTLGNLGYRASAKKAQICQKQ |
| | VKYLGYLLKEGQRWLTEARKETVMGQPTPKTPRQL |
| | REFLGKAGFCRLFIPGFAEMAAPLYPLTKPGTLFN |
| | WGPDQQKAYQEIKQALLTAPALGLPDLTKPFELFV |
| | DEKQGYAKGVLTQKLGPWRRPVAYLSKKLDPVAAG |
| | WPPCLRMVAAIAVLTKDAGKLTMGQPLVILAPHAV |
| | EALVKQPPDRWLSNARMTHYQALLLDTDRVQFGPV |
| | VALNPATLLPLPEEGLQHNCLDILAEAHGTRPDLT |
| | DQPLPDADHTWYTDGSSLLQEGQRKAGAAVTTETE |
| | VIWAKALPAGTSAQRAELIALTQALKMAEGKKLNV |
| | YTDSRYAFATAHIHGEIYRRRGWLTSEGKEIKNKD |
| | EILALLKALFLPKRLSIIHCPGHQKGHSAEARGNR |
| | MADQAARKAAITETPDTSTLLIENSSPSGGSKRTA |
| | DGSEFEPKKKRKV |

TABLE 9
SgRNA common scaffold
and variable spacer sequences.

| Common scaffold | GUUUUAGAGCUAGAAAUAGC |
|---|---|
| sequence (5′-3′) | AAGUUAAAAUAAGGCUAGUC |
| (SEQ ID NO: 106) | CGUUAUCAACUUGAAAAAGU |
| | GGCACCGAGUCGGUGC |
| | sgRNA spacer |
| Locus | sequence (5′-3′) |
| TLR-MCV1 reporter | AAGUUCAGCGUGUCCGGCUU |
| (SEQ ID NO: 54) | |
| hEMX1, +5 G to T | GAGUCCGAGCAGAAGAAGAA |
| (SEQ ID NO: 55) | |
| hHEXA, +1TATC ins | UACCUGAACCGUAUAUCCUA |
| (SEQ ID NO: 56) | |
| hIDUA, +5 G to A | CCGCAGAUGAGGAGCAGCUC |
| (SEQ ID NO: 57) | |
| hHBB, +4-5 AG to TA | CAUGGUGCACCUGACUCCUG |
| (SEQ ID NO: 58) | |
| hVEGFA, +2 G to C | GAUGUCUGCAGGCCAGAUGA |
| and +4-5 GG to CT | |
| (SEQ ID NO: 59) | |
| hRUNX1, +5 G to T | GCAUUUUCAGGAGGAAGCGA |
| (SEQ ID NO: 60) | |
| hPSEN1, +6 G to A | AAAGAGCAUGAUCACAUGCU |
| (SEQ ID NO: 61) | |
| hIDS, +5 G to A | ACUGAGGGAUGUCUGAAGGC |
| (SEQ ID NO: 62) | |
| hFANCF, +2 C to T | GGAAUCCCUUCUGCAGCACC |
| and +4-5 TG to AC | |
| (SEQ ID NO: 63) | |
| hPRNP, +6 G to T | GCAGUGGUGGGGGGCCUUGG |
| (SEQ ID NO: 64) | |
| hDNMT1, +5 G to T | GAUUCCUGGUGCCAGAAACA |
| (SEQ ID NO: 65) | |
| mChd2, +6 G to C | GCGGUAGCUCCCAGAACGGU |
| (SEQ ID NO: 66) | |
| mCol12a1, +5-6 GG | UGACUUCCAUGGUUCCACAA |
| to CC | |
| (SEQ ID NO: 67) | |
| mDNMT1, +6 G to C | CGGGCUGGAGCUGUUCGCGC |
| (SEQ ID NO: 68) | |
| mHEXA, +1 TATC ins | UACCUGAACCGUGUAAAGUA |
| (SEQ ID NO: 69) | |
| mPCSK9, +1 TTAC ins | CCCAUACCUUGGAGCAACGG |
| (SEQ ID NO: 70) | |

TABLE 10
Nicking sgRNA variable spacer sequences.

| | Nicking-sgRNA spacer |
|---|---|
| Locus | sequence (5′-3′) |
| hEMX1, +5 G to T | GACAUCGAUGUCCUCCCCAU |
| (SEQ ID NO: 71) | |
| hHEXA, +1TATC ins | GCUUUCACCUUCAAAUGCCA |
| (SEQ ID NO: 72) | |
| hIDUA, +5 G to A | GGCCGGGCCCUGGGGGCGGU |
| (SEQ ID NO: 73) | |
| hHBB, +4-5 AG to TA | CCUUGAUACCAACCUGCCCA |
| (SEQ ID NO: 74) | |
| hVEGFA, +2 G to C | AUGUACAGAGAGCCCAGGGC |
| and +4-5 GG to CT | |
| (SEQ ID NO: 75) | |
| hRUNX1, +5 G to T | UCACCUCUCAUGAAGCACUG |
| (SEQ ID NO: 76) | |
| hPSEN1, +6 G to A | UUAUCUAAUGGACGACCCCA |
| (SEQ ID NO: 77) | |
| hIDS, +5 G to A | GCAUUUUCGAUUCCGUGACU |
| (SEQ ID NO: 78) | |
| hFANCF, +2 C to T | GGGGUCCCAGGUGCUGACGU |
| and +4-5 TG to AC | |
| (SEQ ID NO: 79) | |
| hPRNP, +6 G to T | GCAUGUUUUCACGAUAGUAA |
| (SEQ ID NO: 80) | |
| hDNMT1, +5 G to T | UUUCCCUUCAGCUAAAAUAA |
| (SEQ ID NO: 81) | |
| mChd2, +6 G to C | GGUGACCGGAGGCAUAUGGA |
| (SEQ ID NO: 82) | |
| mCol12a1, +5-6 | CCAGCCCAGCACAUAAUGGA |
| GG to CC | |
| (SEQ ID NO: 83) | |
| mDNMT1, +6 G to C | GUCGUCUGCAACCUGCAAGA |
| (SEQ ID NO: 84) | |
| mHEXA, +1 TATC ins | GCUCUCACCAUGAAACGCCA |
| (SEQ ID NO: 85) | |
| mPCSK9, +1 TTAC ins | GUUGGGGUGAUGCUCUUCGG |
| (SEQ ID NO: 86) | |

TABLE 11
petRNA sequences
(MS2 sequence + NPT PBS sequences).

| MS2 sequence | GCACAUGAGGAUCACCCAUGUGC |
|---|---|
| (SEQ ID NO: 87) | |
| Locus | NPT-PBS sequence (5′-3′) |
| TLR-MCV1 reporter | UUUGUCUCGCCAAAGGUCUCCGGACACG |
| +1 AGAC ins for | CU |
| mCherry | |
| (SEQ ID NO: 88) | |
| TLR-MCV1 reporter | GUGCAGAUGAACUUCAGGGUCAGCUUGC |
| 39 bp replaced by | CGUAGGUGGCAUCGCCCUCGCCCUCGCC |
| 18 bp for eGFP | GGACACGCU |
| (SEQ ID NO: 89) | |
| hEMX1, +5 G to T | GUGAUGGGAGCACUUCUUCUUCUGCUCG |
| (SEQ ID NO: 90) | GA |
| hHEXA, +1TATC ins | AGUCAGGGCCAUAGGAUAGAUAUACGGU |
| (SEQ ID NO: 91) | UC |
| hIDUA, +5 G to A | ACACUUCGGCCUAGAGCUGCUCCUCAUC |
| (SEQ ID NO: 92) | |
| hHBB, +4-5 AG to TA | AGACUUCUCUACAGGAGUCAGGUGCAC |
| (SEQ ID NO: 93) | |
| hVEGFA, +2 G to C | AAUGUGCCAUCUGGAGCAGUGAUCUGGC |
| and +4-5 GG to CT | CUGCAGA |
| (SEQ ID NO: 94) | |
| hRUNX1, +5 G to T | UGUCUGAAGCAAUCGCUUCCUCCUGAAA |
| (SEQ ID NO: 95) | AU |
| hPSEN1, +6 G to A | AAAUAUGGCGUCAAGCAUGUGAUCAUGC |
| (SEQ ID NO: 96) | UCU |
| hIDS, +5 G to A | GCCAGUAUCCCUGGCCUUCAGACAUCCC |
| (SEQ ID NO: 97) | U |
| hFANCF, +2 C to T | GGAAAAGCGAUCGUGAUGCUGCAGAAGG |
| and +4-5 TG to AC | GAU |
| (SEQ ID NO: 98) | |
| hPRNP, +6 G to T | AUGUAGACGCCAAGGCCCCCCACC |
| (SEQ ID NO: 99) | |
| hDNMT1, +5 G to T | UCCCGUCACCACUGUUUCUGGCACCAGG |
| (SEQ ID NO: 100) | |
| mChd2, +6 G to C | GAUGGCCACCGUUCUGGGAGCUA |
| (SEQ ID NO: 101) | |
| mCol12a1, +5-6 GG to CC | AAUGGACGGAUUGUGGAACCAUGGAA |
| (SEQ ID NO: 102) | |
| mDNMT1, +6 G to C | AAGAUGGCAGCGCGAACAGCUCCAG |
| (SEQ ID NO: 103) | |
| mHEXA, +1 TATC ins | AGUCAGGGCCAUACGAUAUUUACACGGU |
| (SEQ ID NO: 104) | UC |
| mPCSK9, +1 TTAC ins | ACCGCCACCUUCCGCCGGUAAUUGCUCC |
| (SEQ ID NO: 105) | AAGGUAU |

### EXAMPLES

### Example 1: Construction, Purification, and Transfection of Plasmids and Genomic DNA Isolation

### Plasmid Construction

A strain of split prime editor guide RNA pegRNA expression plasmids were constructed by HiFi DNA assembly (NEB) of vector backbone (enzyme-digested or PCR product) and gBlock fragments (IDT). sgRNA, nicking-sgRNA, and ribozyme-flanked petRNA expression plasmids were generated by HiFi DNA assembly of single-stranded oligonucleotides (IDT) and vector backbone (PCR product). Effector expression plasmids were constructed by HiFi DNA assembly of vector backbone (digested at corresponding position of PE2 plasmid) and inserts (gBlock or PCR fragments). Plasmids were confirmed by Sanger sequencing or Whole plasmid sequencing (Plasmidsaurus). Plasmids were purified using a Miniprep or Midiprep kit (Promega) for cellular experiments.

### Cell Culture, Transfection, and Genomic DNA Isolation

PegRNA expression plasmids were constructed by HiFi DNA assembly (NEB) of vector backbone (enzyme-digested or PCR product) and gBlock fragments (IDT). sgRNA, nicking-sgRNA, and ribozyme-flanked petRNA expression plasmids were generated by HiFi DNA assembly of single-stranded oligonucleotides (IDT) and vector backbone (PCR product). Effector expression plasmids were constructed by HiFi DNA assembly of vector backbone (digested at corresponding position of PE2 plasmid) and inserts (gBlock or PCR fragments). Plasmids were confirmed by Sanger sequencing or Whole plasmid sequencing (Plasmidsaurus). Plasmids were purified using a Miniprep or Midiprep kit (Promega) for cellular experiments.

### Electroporation

Neon electroporation system was used. pegRNAs, sgRNAs, and nicking sgRNAs were ordered from IDT with chemical modifications. petRNAs were either ordered from IDT or synthesized in-house. Briefly, 500 ng of each mRNA, 50 pmol sgRNA+50 pmol petRNA (or 50 pmol pegRNAs), and 25,000 TLR-MCV1 reporter cells were mixed in Buffer R and electroporated using 10-μl Neon tips using the following electroporation parameters: 1, 150 V, 20 ms, two pulses. After electroporation, cells were plated in prewarmed 96-well plates with DMEM containing 10% FBS and incubated for 72 h before analysis.

### Example 2: Deep Sequencing and Data Analysis, In Vitro Transcription for mRNA Production, and Flow Cytometry Analysis

### Deep Sequencing and Data Analysis

Neon electroporation system was used. pegRNAs, sgRNAs, and nicking sgRNAs were ordered from IDT with chemical modifications. petRNAs were either ordered from IDT or synthesized in-house. Briefly, 500 ng of each mRNA, 50 pmol sgRNA+50 pmol petRNA (or 50 pmol pegRNAs), and 25,000 TLR-MCV1 reporter cells were mixed in Buffer R and electroporated using 10-μl Neon tips using the following electroporation parameters: 1, 150 V, 20 ms, two pulses. After electroporation, cells were plated in prewarmed 96-well plates with DMEM containing 10% FBS and incubated for 72 h before analysis.

In Vitro Transcription for mRNA Production

In-vitro transcription template plasmids were constructed by adding a CleanCap Reagent AG-compatible T7 promoter (TAATACGACTCACTATAAG) (SEQ ID NO:113) and a 5′-UTR were inserted at the 5′ end of the Kozak sequence of the coding sequence, and also adding A 3′-UTR, a 110-nt poly(A) (SEQ ID NO:114) tract and a restriction site (Esp3I) after the stop codon. Plasmids were completely linearized using Esp3I (NEB) for in-vitro transcription, which was performed at 37° C. using a HiScrib T7 High Yield RNA Synthesis kit (NEB) with the addition of CleanCap Reagent AG (Trilink) for Cap1 structure and with a 100% replacement of UTP by NI-Methylpseudo-UTP (Trilink). The reaction was terminated after 2 h by a 15-min incubation with DNase I (NEB). The RNA was then purified using a Monarch RNA Cleanup kit (NEB).

### Flow Cytometry Analysis

Flow cytometry analysis was performed on day 3 after transfection or electroporation. TLR-MCV1 reporter cells were collected after trypsin digestion and then resuspended in PBS with 2% FBS. The mCherry or GFP positive cells were quantified using flow cytometry (MACSQuant VYB). Data were analyzed by FlowJo v10 software.

### Example 3: In Vitro Evaluation of Prime Editing Efficiencies of PEs Prime Editing Constructs with the Fusion Protein Comprising One or More MS2 Binding Proteins Inlaid within Cas9, “Inlaid Fusion Proteins”

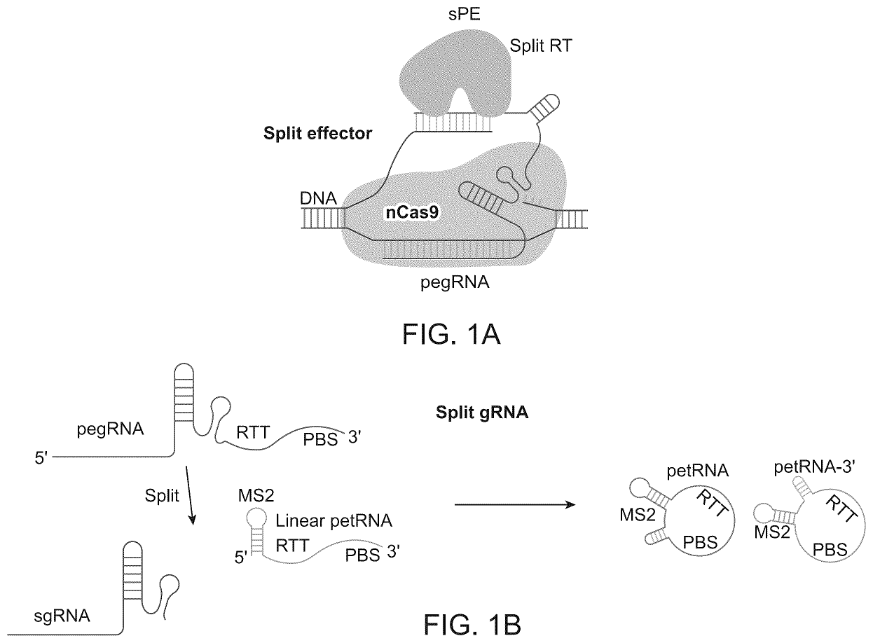

Modular primer editing effectors designed previously to have split effectors (i.e. untethered Cas9 H840A nickase or H840A, and nucleotide polymerase template or RT; FIG. 1) or to have split prime editing gRNAs (split into a single guide RNA or sgRNA and modified prime editing templates or petRNA; FIGS. 2A-2C). These modular prime editing systems produced inadequate editing efficiencies and required optimization. Therefore, novel prime editing systems were engineered containing a single fused effector (fusion protein) comprised of a Cas9 nickase linked to an RT, a petRNA comprising a PBS, an NPT, and at least one MS2 hairpin, and an sgRNA, wherein the fusion protein comprises at least one MS2 binding protein inlaid within the Cas9 nickase at one or more favorable positions for a better recruitment of the petRNA.

Fusion proteins with one or more inlaid MCPs at several positions within the Cas9 nickase of SEQ ID NO: 1 were designed. These positions within the Cas9 nickase of SEQ ID NO: 1 include: the Rec-I lobe, nuclease domains HNH and RuvC-III, the PAM-interacting domain (PID) of the Cas9 nickase. The one or more MCP were inserted at position S355 at the Rec-I domain of the Cas9 nickase sequence of SEQ ID NO: 1, at positions E1026 and N1054 of the RuvC-III domain of the Cas9 nickase sequence of SEQ ID NO: 1, at positions G1247 and D1299 of the PID domain of the Cas9 nickase sequence of SEQ ID NO: 1, and at positions E827 and delta S793-R905 of the HNH domain of the Cas9 nickase sequence of SEQ ID NO: 1.

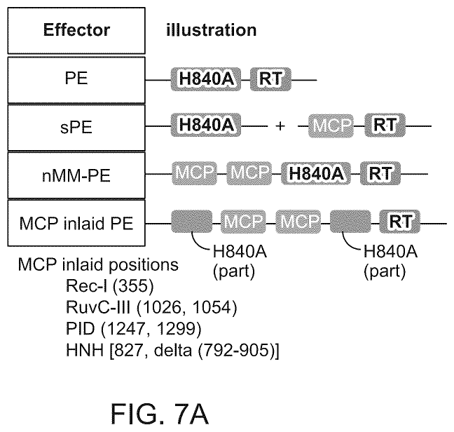

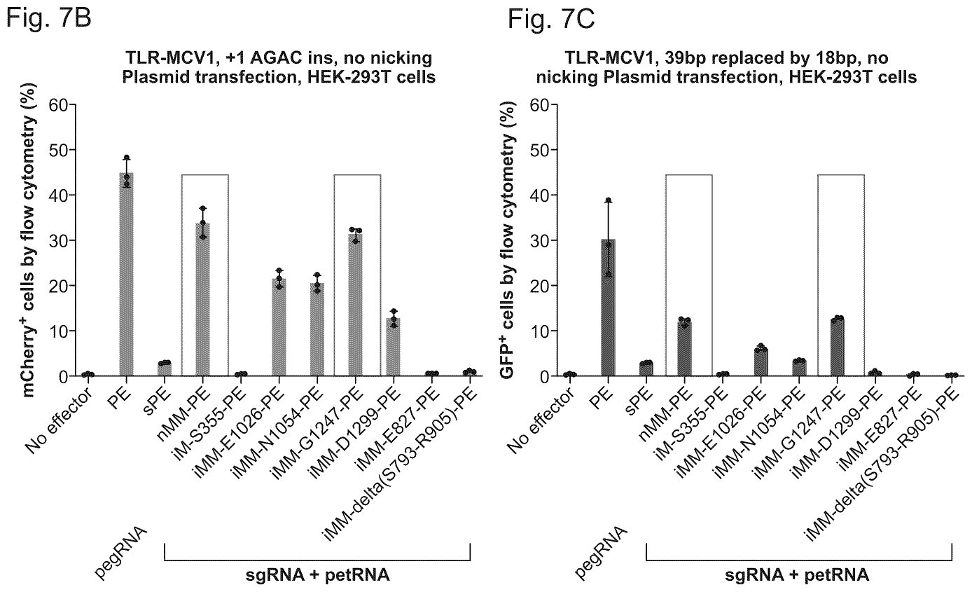

To evaluate eukaryotic cell DNA repair outcomes of these inlaid modular prime editing effectors, a “traffic light reporter” (TLR-MCV1) locus in reporter plasmids encoding EGFP and mCherry was used to perform edits in vitro using the methods described in example 1. The reporter plasmids then transform the reaction products into yeast cells. The editing efficiencies of these inlaid prime editing constructs were then systematically evaluated and compared to conventional PE (no MCP), and an sPE (split effector with an MCP fused to the NP). The inlaid variants tested were: iM-S355-PE, iMM-E1026-PE, iMM-N1054-PE, iMM-G1247-PE. iMM-D1299-PE. iMM-E827-PE and iMM-delta (S793-R905)-PE). MCP were inserted at the Rec-I (355), RuvC-III (1026, 1054), PID (1247, 1299), and HNH [827, delta (792-905)] domain of the Cas9 nickase of SEQ ID NO: 1, respectively. These prime editing constructs were tested for their ability to install a +1 AGAC sequence insert (FIG. 7B) or to replace a 39 bp sequence by an 18 bp sequence (FIG. 7C) without a nicking sgRNA in a TLR-MCV1 locus in HEK-293T cells. Among the tested inlaid constructs, the inlaid construct iMM-G1247-PE exhibited the best activity.

Prime Editing Constructs with the Fusion Protein Comprising at Least Four MS2 Binding Proteins.

Next, the efficiency of modular prime editing systems wherein the fusion protein comprised at least four MS2 binding proteins were tested. MCP has been suggested to be an obligate homodimer. Therefore, the use of MCP dimers or multimers instead of an MCP monomer may improve binding and recruitment of petRNA.

Effectors comprising at least four MS2 binding proteins were designed. These included: nMMM-PE which comprised of 4 MS2 binding proteins on the N terminus of the nCas9 (FIG. 8, effector nMMM-PE), and nMMcMM, which comprised of an MCP dimer on each terminal (2 MS2 binding proteins on the N terminus of the nCas9 and 2 binding proteins on the C terminus of the nCas9 of the RT).

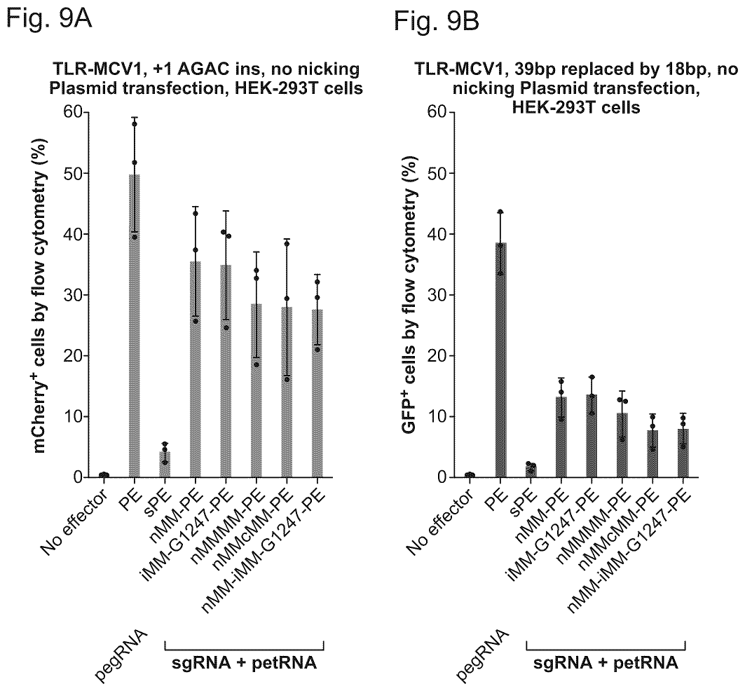

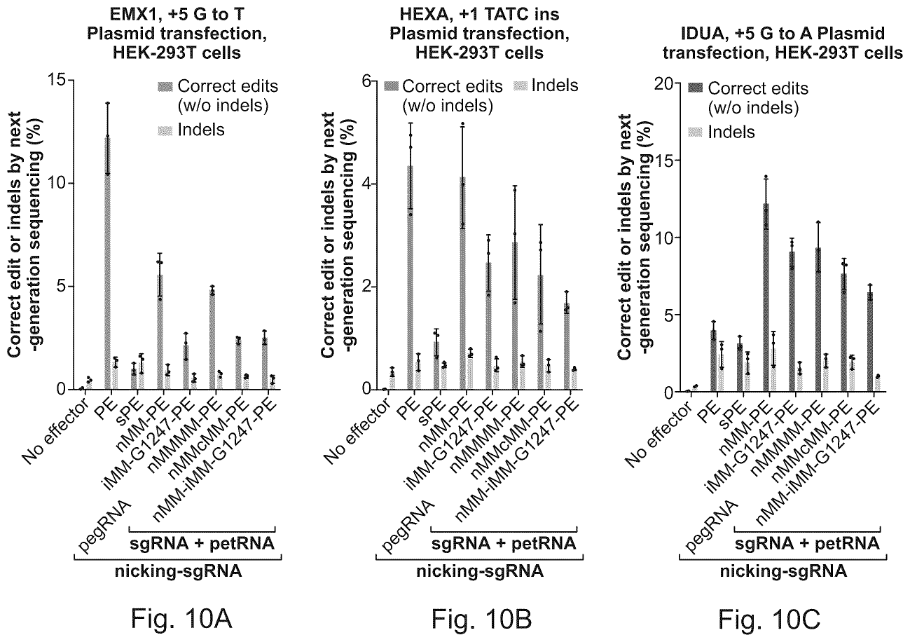

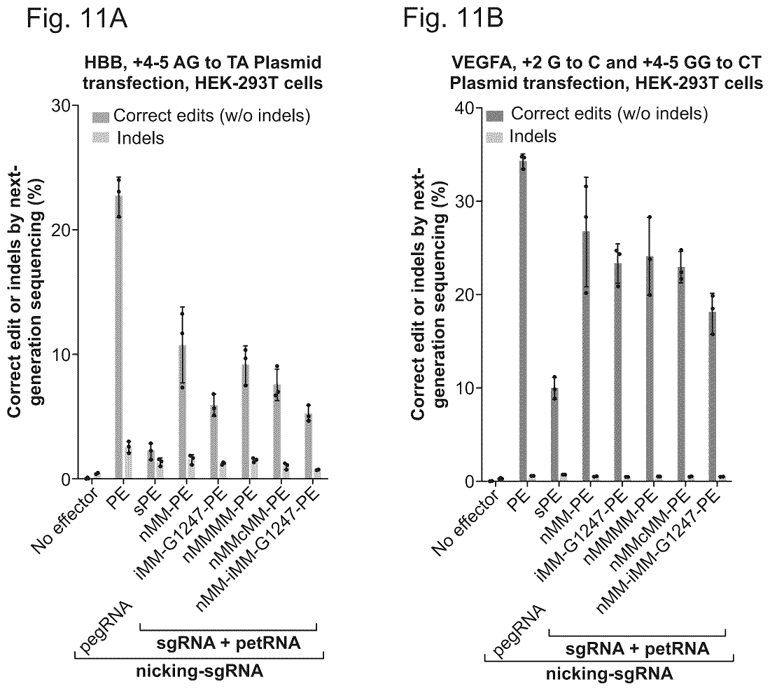

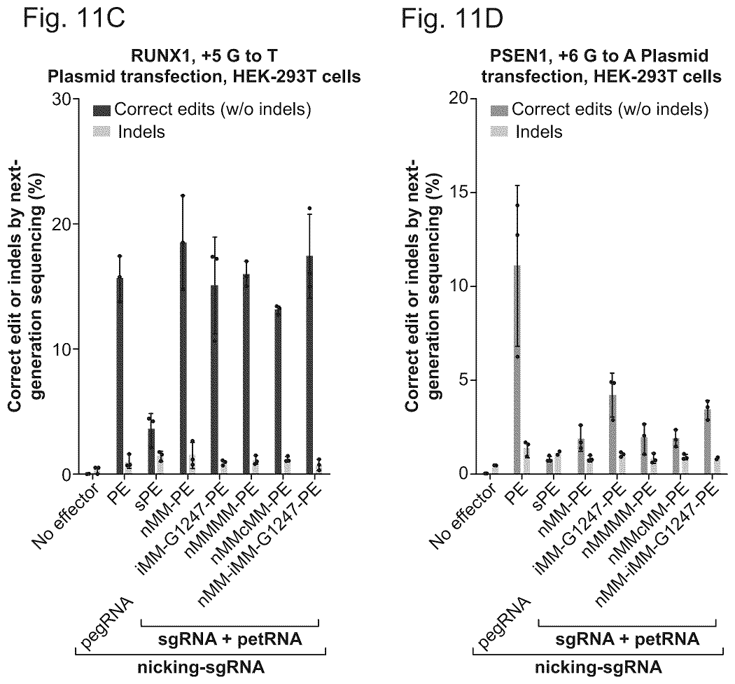

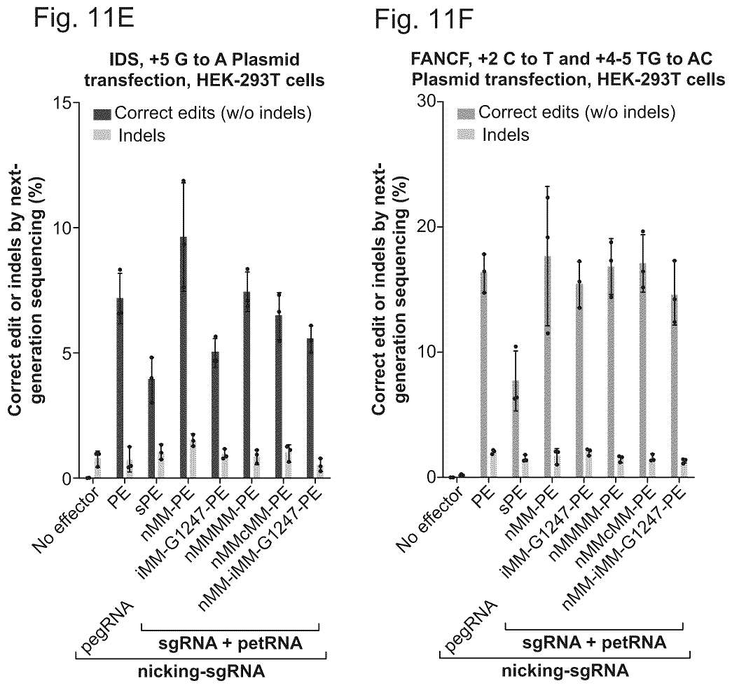

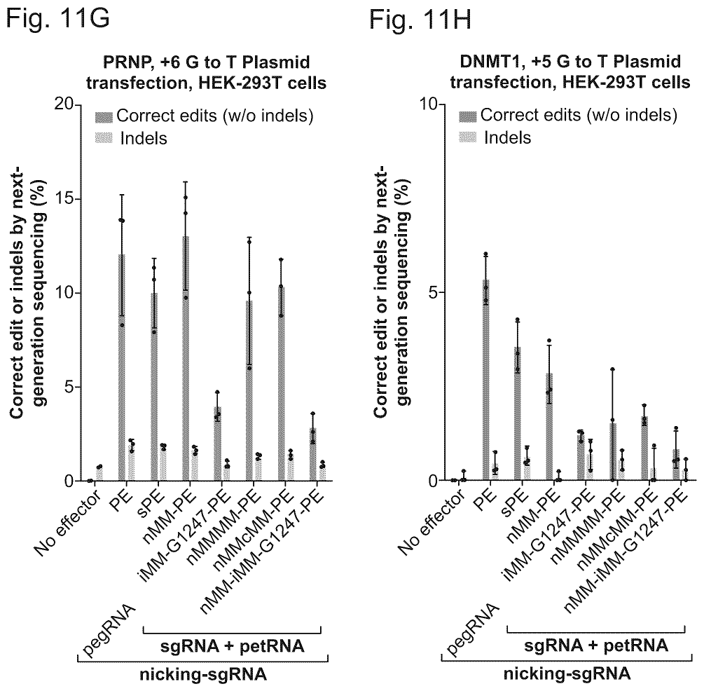

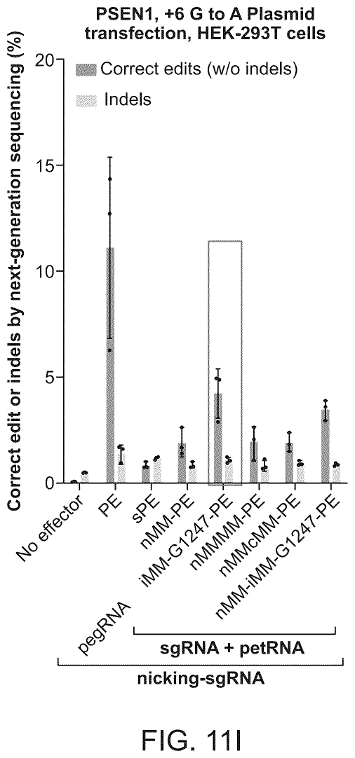

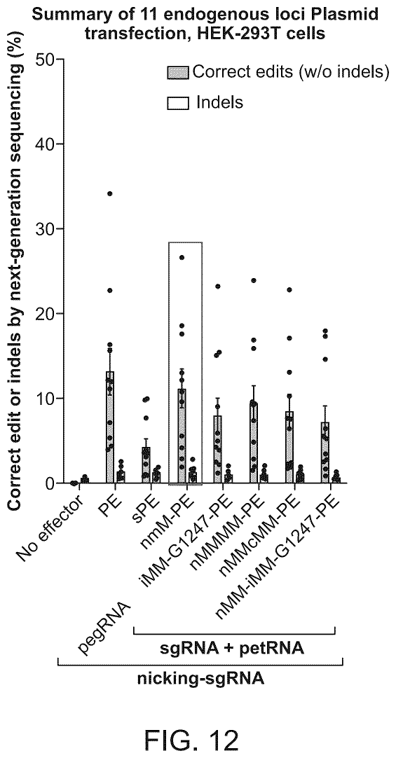

These PEs were investigated for their ability to install a +1 “AGAC” sequence insert (FIG. 9A), to replace a 39 bp sequence by an 18 bp sequence (FIG. 9B), to insert a +5 G to T edit at the EMX1 locus (FIG. 10A), a +1 TATC insert in HEXA (FIG. 10B), a +5 G to A edit in IDUA (FIG. 10C), to insert a +4-5 A·G-to-T·A edit in HBB using the (FIG. 11A), a +2 G to C and a +4-5 G·G-to-C·T edit in VEGFA (FIG. 11B), a +5 G to T edit in RUNX1 (FIG. 11C), a +5 G to T edit in PSEN1 (FIG. 11D), A+5 G to A edit in IDS is (FIG. 11E), a +2 C to T and a +4-5 T·G-to-A·C edit in FANCF (FIG. 11F), a +6 G to T edit in PRNP (FIG. 11G), a +5 G to T edit in DNMT1 (FIG. 11H), and a +6 G to A edit in PSEN1 (FIG. 11I).

These data were directly compared to that of PE and sPE. Effectors nMMM-PE exhibited better editing efficiencies than nMMcMM in most experiments, and, in most experiments, these effectors showed an equivalent or a smaller amount of indels as compared to PE and sPE prime editors.

To further optimize these effectors, a prime editor effector was designed to comprise both feature of the inlaid concept and the fusion protein comprising at least four MCPs. This effector, nMM-iMM-G1247-PE (FIG. 8) comprised an MCP dimer on the N-terminus of the nCas9 and an MCP dimer inlaid within the G1247 position of the nCas9 sequence of SEQ ID: NO: 1. The inlaid position G1247 was chosen because the inlaid construct iMM-G1247-PE exhibited the best editing efficiency.

The efficiency of this effector was directly compared to that of PE, sPE, nMMM-PE, and nMMcMM for its ability to install a +1 “AGAC” sequence insert (FIG. 9A), to replace a 39 bp sequence by an 18 bp sequence (FIG. 9B), to insert a +5 G to T edit at the EMX1 locus (FIG. 10A), a +1 TATC insert in HEXA (FIG. 10B), a +5 G to A edit in IDUA (FIG. 10C), to insert a +4-5 A·G-to-T·A edit in HBB using the (FIG. 11A), a +2 G to C and a +4-5 G·G-to-C·T edit in VEGFA (FIG. 11B), a +5 G to T edit in RUNX1 (FIG. 11C), a +5 G to T edit in PSEN1 (FIG. 11D), A+5 G to A edit in IDS is (FIG. 11E), a +2 C to T and a +4-5 T·G-to-A·C edit in FANCF (FIG. 11F), a +6 G to T edit in PRNP (FIG. 11G), a +5 G to T edit in DNMT1 (FIG. 11H), and a +6 G to A edit in PSEN1 (FIG. 11I).

The editing efficiency of nMM-iMM-G1247-PE varied, with some edits showing similar editing efficiencies to that of nMMM-PE and nMMcMM, and some improved editing efficiencies than that of nMMM-PE and nMMcMM. In almost all instances, the indels generated by nMM-iMM-G1247-PE were equal or less than those generated by nMMM-PE and nMMcMM.

Prime Editing Constructs with the Fusion Protein Comprising at Least One MS2 Binding Protein at the N Terminus and at Least One MS2 Binding Protein at the C Terminus.

Next, the efficiency of modular prime editing systems wherein the fusion protein comprised at least one MS2 binding protein at the N terminus and at least one MS2 binding protein at the C terminus were tested.

An effector comprising one MS2 binding protein at the N terminus of the nCas9 and one MS2 binding protein at the C terminus of the RT was designed (A schematic with effector nMcM-PE can be seen in FIGS. 2A-2C).

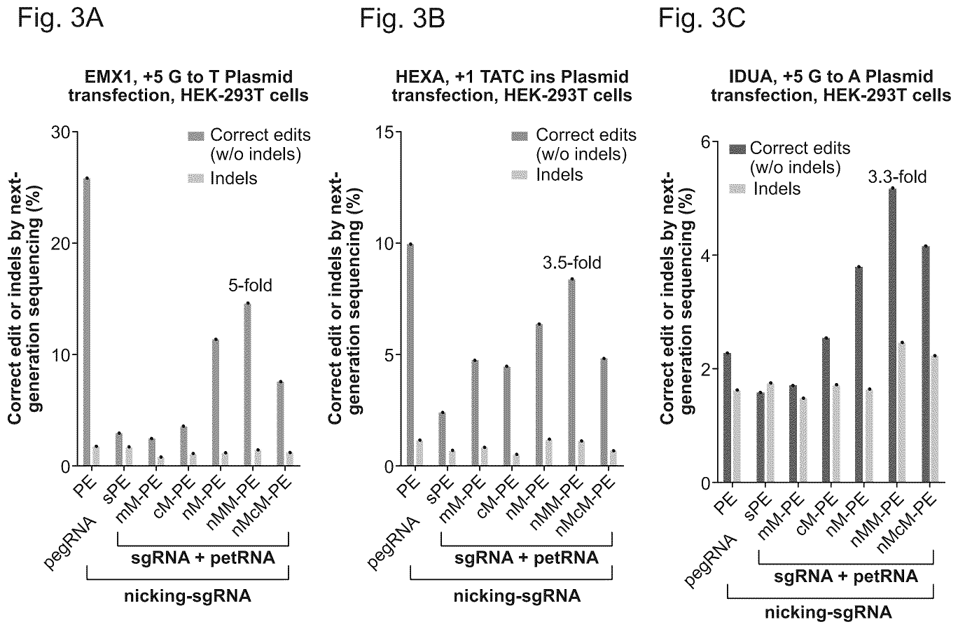

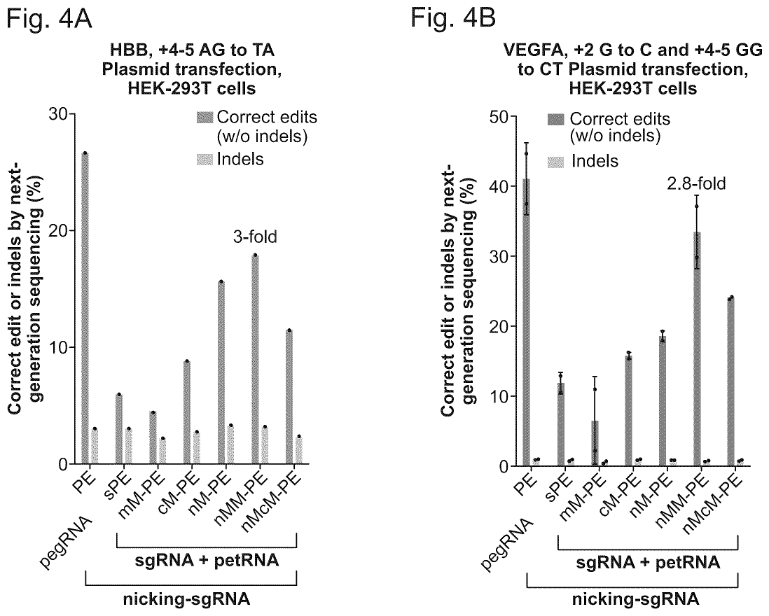

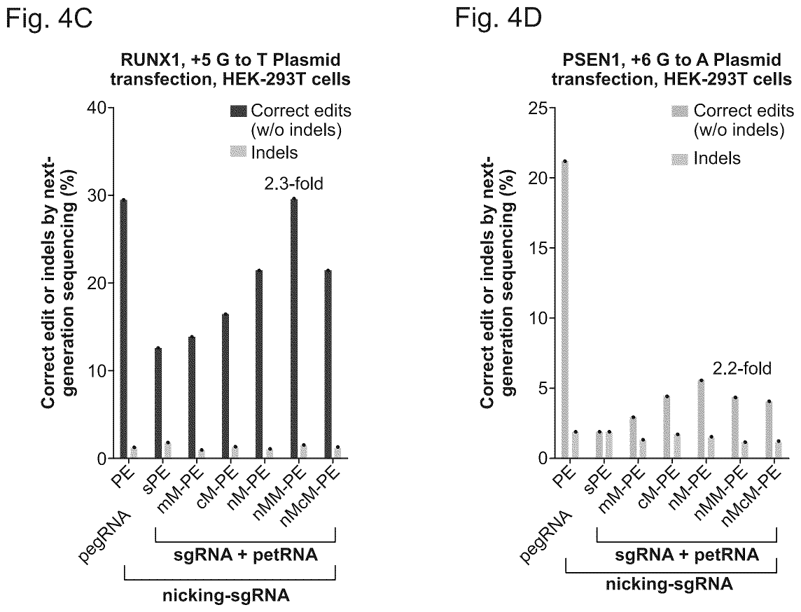

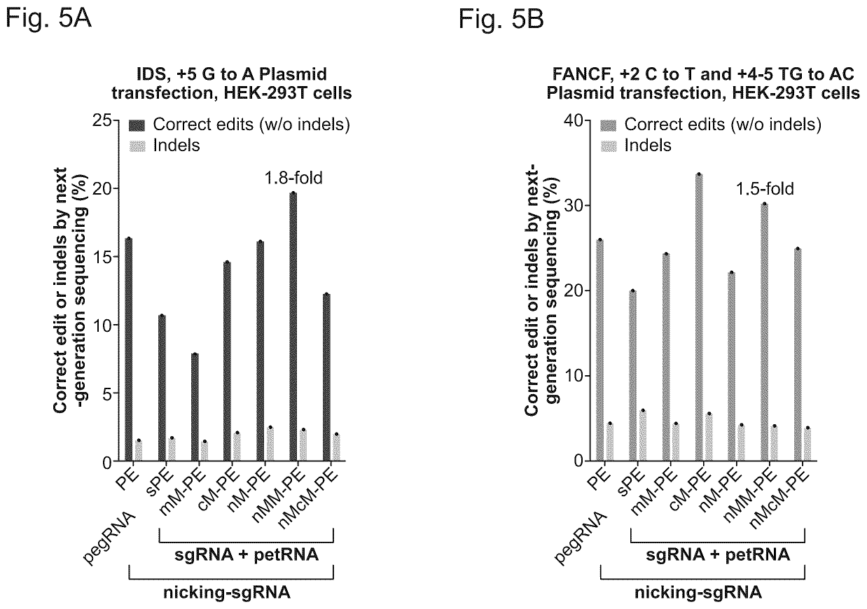

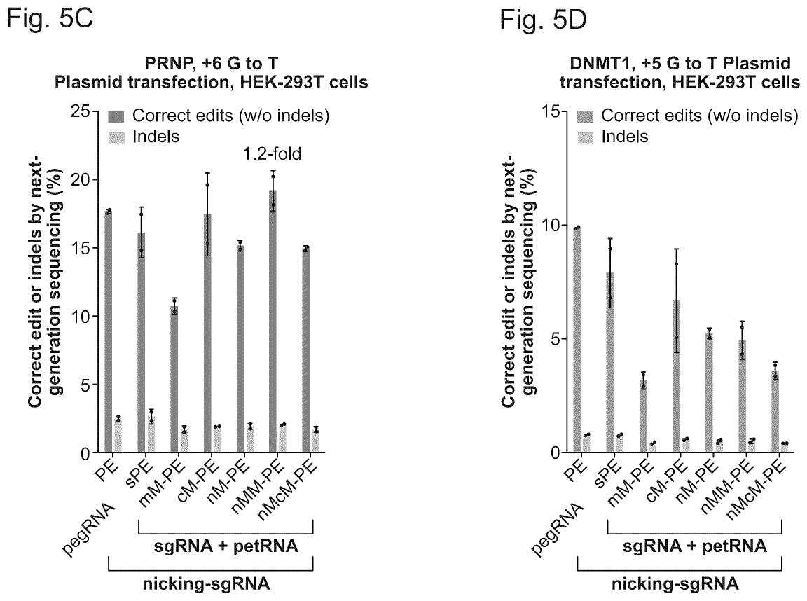

nMcM was investigated for its ability to install a +1 “AGAC” sequence insert (FIG. 2B), to replace a 39 bp sequence by an 18 bp sequence (FIG. 2C), a +5 G to T edit in EMX1 (FIG. 3A), a +1 TATC insert in HEXA (FIG. 3B), and a +5 G to A edit in IDUA (FIG. 3C), to insert a +4-5 A·G-to-T·A edit in HBB using the (FIG. 4A), a +2 G to C and a +4-5 G·G-to-C·T edit in VEGFA (FIG. 4B), a +5 G to T edit in RUNX1 (FIG. 4C), a +5 G to T edit in PSEN1 (FIG. 4D), A+5 G to Ax edit in IDS is (FIG. 5A), a +2 G to C a +4-6 G·G-to-C·T edit in FANCF (FIG. 5B), a +6 G to T edit in PRNP (FIG. 5C), and a +6 G to T edit in DNMT1 (FIG. 5D).

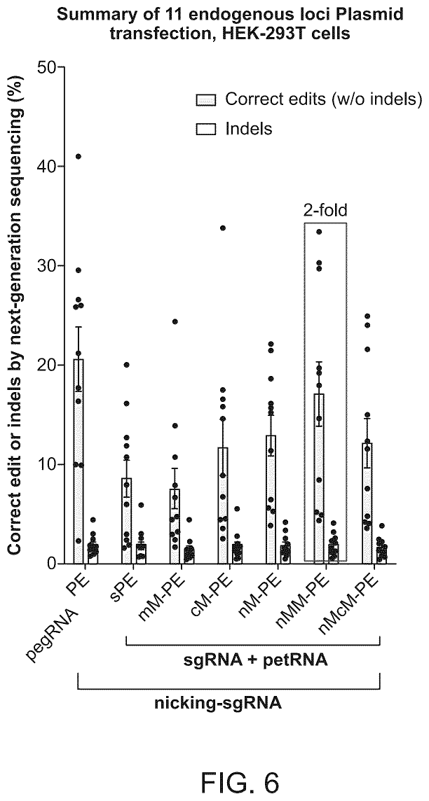

These data were directly compared to that of PE and sPE. The editing efficiency of effector nMcM-PE varied. However, in almost all instances, nMcM-PE showed an improved editing efficiency at 11 endogenous loci in HEK-293T cells as opposed to sPE (FIG. 6).

Prime Editing Constructs with the Fusion Protein Comprising at Least One MS2 Binding Protein at the N Terminus or at Least One MS2 Binding Protein at the C Terminus or at Least One MS2 Binding Protein Between the Cas9 Nickase and the RT.

Next, the efficiency of modular prime editing systems wherein the fusion protein comprised at least one MS2 binding protein at the N terminus or at least one MS2 binding protein at the C terminus or at least one MS2 binding protein between the Cas9 nickase and the RT were tested.

Effector mM-PE was designed with one MCP between the nCas9 and the RT, effector cM-PE with one MCP on the C terminus of the RT, effector nM-PE with one MCP on the N terminus of the nCas9, and nMM-PE with an MCP dimer on the N terminus (FIGS. 2A-2C).

These PEs were investigated for their ability to install a +1 “AGAC” sequence insert (FIG. 2B), to replace a 39 bp sequence by an 18 bp sequence (FIG. 2C), a +5 G to T edit in EMX1 (FIG. 3A), a +1 TATC insert in HEXA (FIG. 3B), and a +5 G to A edit in IDUA (FIG. 3C), to insert a +4-5 A·G-to-T·A edit in HBB using the (FIG. 4A), a +2 G to C and a +4-5 G·G-to-C·T edit in VEGFA (FIG. 4B), a +5 G to T edit in RUNX1 (FIG. 4C), a +5 G to T edit in PSEN1 (FIG. 4D), A+5 G to Ax edit in IDS is (FIG. 5A), a +2 G to C a +4-6 G·G-to-C·T edit in FANCF (FIG. 5B), a +6 G to T edit in PRNP (FIG. 5C), and a +6 G to T edit in DNMT1 (FIG. 5D).

These data were directly compared to that of PE, sPE, and nMcM-PE. Effector nMM-PE, on average showed an editing efficiency at least 2-fold better than that of sPE at 11 endogenous loci in HEK-293T cells (FIG. 6), with editing efficiencies having up to 9-fold improvement over sPE.

### Optimized Editing Efficiency Summaries

Overall, among tested effectors, the N-terminal MCP-dimer fused PE (nMM-PE) has a 2-fold improvement in editing efficiency over sPEs on average when tested on 11 endogenous loci. This prime editing is comparable to the canonical pegRNA-based prime editing. As for the inserted (inlaid) positions, inlaid position G1247 of the nCas9 sequence of SEQ ID NO: 1 (iMM-G1247-PE) exhibited activity as good as the N-terminally fused configuration (nMM-PE).

### Chemical Modification of the PetRNA

To further optimize editing efficiencies of these PE constructs, novel linkers were used to link the MS2 coat protein to the NPT primer binding site (NPT-PBS) sequence to see their effects on editing efficiencies. 2′-Omethyl (2′-OMe) modified RNA linkers (including a fully modified 2′-OMe RNA linker, AC7), and a 2× hexaethylene glycol (2×HEG) linker were among the linkers tested.

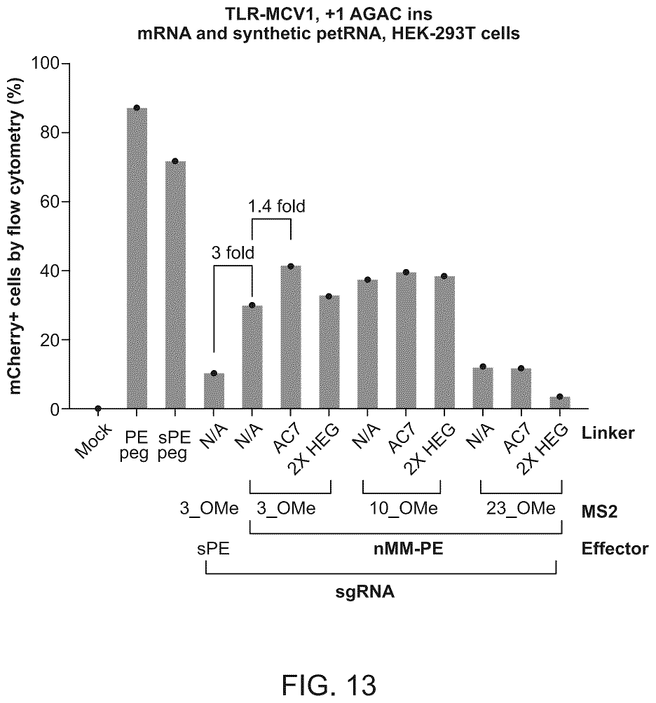

As shown in FIG. 13, The fully 2′-OMe modified linker, AC7, improved editing by ˜1.4 fold. Additional benefits of using a 2′-OMe modified linker include reduced readthrough activity and improved stability in vivo. Sequences of the tested petRNAs are shown below in Table 12.

TABLE 12
petRNA sequences

| MS2 | Linker | RTT | PBS |
|---|---|---|---|
| (mG)#(mC)#(mA)# | N/A | UUUGUCUCGCCA | (mC)(mC)(mG)(mG) |
| CAUGAGGAUCACCCA | | AAGGUCU | (mA)(mC)(mA)(mC)# |
| UGUGC | | (SEQ ID NO: 110) | (mG)#(mC)#(mU) |
| (SEQ ID NO: 107) | | | (SEQ ID NO: 111) |
| (mG)#(mC)#(mA)# | AC7 (OMe) | UUUGUCUCGCCA | (mC)(mC)(mG)(mG) |
| CAUGAGGAUCACCCA | | AAGGUCU | (mA)(mC)(mA)(mC)# |
| UGUGC | | (SEQ ID NO: 110) | (mG)#(mC)#(mU) |
| (SEQ ID NO: 107) | | | (SEQ ID NO: 111) |
| (mG)#(mC)#(mA)# | 2X HEG | UUUGUCUCGCCA | (mC)(mC)(mG)(mG) |
| CAUGAGGAUCACCCA | | AAGGUCU | (mA)(mC)(mA)(mC)# |
| UGUGC | | (SEQ ID NO: 110) | (mG)#(mC)#(mU) |
| (SEQ ID NO: 107) | | | (SEQ ID NO: 111) |
| (mG)#(mC)#(mA)# | N/A | UUUGUCUCGCCA | (mC)(mC)(mG)(mG) |
| (mC)(mA)(mU)(mG) | | AAGGUCU | (mA)(mC)(mA)(mC)# |
| (mA)(mG)(mG)AUC | | (SEQ ID NO: 110) | (mG)#(mC)#(mU) |
| ACCCAUGUGC | | | (SEQ ID NO: 111) |
| (SEQ ID NO: 108) | | | |
| (mG)#(mC)#(mA)# | AC7 (OMe) | UUUGUCUCGCCA | (mC)(mC)(mG)(mG) |
| (mC)(mA)(mU)(mG) | | AAGGUCU | (mA)(mC)(mA)(mC)# |
| (mA)(mG)(mG)AUC | | (SEQ ID NO: 110) | (mG)#(mC)#(mU) |
| ACCCAUGUGC | | | (SEQ ID NO: 111) |
| (SEQ ID NO: 108) | | | |
| (mG)#(mC)#(mA)# | 2X HEG | UUUGUCUCGCCA | (mC)(mC)(mG)(mG) |
| (mC)(mA)(mU)(mG) | | AAGGUCU | (mA)(mC)(mA)(mC)# |
| (mA)(mG)(mG)AUC | | (SEQ ID NO: 110) | (mG)#(mC)#(mU) |
| ACCCAUGUGC | | | (SEQ ID NO: 111) |
| (SEQ ID NO: 108) | | | |
| (mG)#(mC)#(mA)# | N/A | UUUGUCUCGCCA | (mC)(mC)(mG)(mG) |
| (mC)(mA)(mU)(mG) | | AAGGUCU | (mA)(mC)(mA)(mC)# |
| (mA)(mG)(mG)(mA) | | (SEQ ID NO: 110) | (mG)#(mC)#(mU) |
| (mU)(mC)(mA)(mC) | | | (SEQ ID NO: 111) |
| (mC)(mC)(mA)(mU) | | | |
| (mG)(mU)(mG)(mC) | | | |
| (SEQ ID NO: 109) | | | |
| (mG)#(mC)#(mA)# | AC7 (OMe) | UUUGUCUCGCCA | (mC)(mC)(mG)(mG) |
| (mC)(mA)(mU)(mG) | | AAGGUCU | (mA)(mC)(mA)(mC)# |
| (mA)(mG)(mG)(mA) | | (SEQ ID NO: 110) | (mG)#(mC)#(mU) |
| (mU)(mC)(mA)(mC) | | | (SEQ ID NO: 111) |
| (mC)(mC)(mA)(mU) | | | |
| (mG)(mU)(mG)(mC) | | | |
| (SEQ ID NO: 109) | | | |
| (mG)#(mC)#(mA)# | 2X HEG | UUUGUCUCGCCA | (mC)(mC)(mG)(mG) |
| (mC)(mA)(mU)(mG) | | AAGGUCU | (mA)(mC)(mA)(mC)# |
| (mA)(mG)(mG)(mA) | | (SEQ ID NO: 110) | (mG)#(mC)#(mU) |
| (mU)(mC)(mA)(mC) | | | (SEQ ID NO: 111) |
| (mC)(mC)(mA)(mU) | | | |
| (mG)(mU)(mG)(mC) | | | |
| (SEQ ID NO: 109) | | | |

For Table 12, “m” corresponds to a 2′-Omethyl modification and “#” corresponds to a phosphorothioate internucleotide linkage.

1. A modular prime editing system, comprising:
i) a fusion protein comprising a Cas9 nickase protein linked to a nucleotide polymerase (NT) protein,
ii) a prime editor template RNA (petRNA) comprising a primer binding site (PBS), a nucleotide polymerase template (NPT), and at least one MS2 hairpin, and
iii) a single guide RNA (sgRNA),
wherein the fusion protein comprises at least one MS2 binding protein inlaid within the Cas9 nickase.

2. The modular prime editing system of claim 1, wherein the fusion protein comprises or consists of two or more MS2 binding proteins inlaid within the Cas9 nickase, optionally wherein the two or more MS2 binding proteins are inlaid within one of the Rec-1, RuvC-III, PID, or HNH domains of the Cas9 nickase.

3-13. (canceled)

14. The modular prime editing system of claim 1, wherein the fusion protein comprises at least two MS2 binding proteins inlaid within position G1247 of the PID domain of the Cas9 nickase of SEQ ID NO: 1.

15-23. (canceled)

24. The modular prime editing system of claim 1, wherein the fusion protein comprises from the N-terminus to the C-terminus:
the N-terminus portion of the Cas9 nickase protein, one MS2 binding protein, the C-terminus portion of the Cas9 nickase protein, and an NT protein; or
the N-terminus portion of the Cas9 nickase protein, two MS2 binding proteins, the C-terminus portion of the Cas9 nickase protein, and an NT protein, optionally wherein the fusion protein comprises from the N-terminus to the C-terminus;
the N-terminus portion of the Cas9 nickase protein, a first linker, one MS2 binding protein, a second linker, the C-terminus portion of the Cas9 nickase protein, a third linker, and an NT protein; or
the N-terminus portion of the Cas9 nickase protein, a first linker, a first MS2 binding protein, a second linker, a second MS2 binding protein, a third linker, the C-terminus portion of the Cas9 nickase protein, a fourth linker, and an NT protein.

25-27. (canceled)

28. The modular prime editing system of claim 24, wherein:
A:
the N-terminus portion of the Cas9 nickase protein comprises the sequence of SEQ ID NO: 2;
the MS2 binding protein comprises the sequence of SEQ ID NO: 21;
the C-terminus portion of the Cas9 nickase protein comprises the sequence of SEQ ID NO: 11; and
the NT protein comprises the sequence of SEQ ID NO: 19; or
B:
the N-terminus portion of the Cas9 nickase protein comprises the sequence of SEQ ID NO: 3;
the first MS2 binding protein comprises the sequence of SEQ ID NO: 21;
the second MS2 binding protein comprises the sequence of SEQ ID NO: 21;
the C-terminus portion of the Cas9 nickase comprises the sequence of SEQ ID NO: 12; and
the NT protein comprises the sequence of SEQ ID NO: 19; or
C:
the N-terminus portion of the Cas9 nickase protein comprises the sequence of SEQ ID NO: 4;
the first MS2 binding protein comprises the sequence of SEQ ID NO: 21;
the second MS2 binding protein comprises the sequence of SEQ ID NO: 21;
the C-terminus portion of the Cas9 nickase comprises the sequence of SEQ ID NO: 13; and
the NT protein comprises the sequence of SEQ ID NO: 19; or
D:
the N-terminus portion of the Cas9 nickase protein comprises the sequence of SEQ ID NO: 5;
the first MS2 binding protein comprises the sequence of SEQ ID NO: 21;
the second MS2 binding protein comprises the sequence of SEQ ID NO: 21;
the C-terminus portion of the Cas9 nickase comprises the sequence of SEQ ID NO: 14; and
the NT protein comprises the sequence of SEQ ID NO: 19; or
E:
the N-terminus portion of the Cas9 nickase protein comprises the sequence of SEQ ID NO: 6;
the first MS2 binding protein comprises the sequence of SEQ ID NO: 21;
the second MS2 binding protein comprises the sequence of SEQ ID NO: 21;
the C-terminus portion of the Cas9 nickase comprises the sequence of SEQ ID NO: 15; and
the NT protein comprises the sequence of SEQ ID NO: 19; or
F:
the N-terminus portion of the Cas9 nickase protein comprises the sequence of SEQ ID NO: 7;
the first MS2 binding protein comprises the sequence of SEQ ID NO: 21;
the second MS2 binding protein comprises the sequence of SEQ ID NO: 21;
the C-terminus portion of the Cas9 nickase comprises the sequence of SEQ ID NO: 16; and
the NT protein comprises the sequence of SEQ ID NO: 19; or
G:
the N-terminus portion of the Cas9 nickase protein comprises the sequence of SEQ ID NO: 8;
the first MS2 binding protein comprises the sequence of SEQ ID NO: 21;
the second MS2 binding protein comprises the sequence of SEQ ID NO: 21;
the C-terminus portion of the Cas9 nickase comprises the sequence of SEQ ID NO: 17; and
the NT protein comprises the sequence of SEQ ID NO: 19; or
H:
the N-terminus portion of the Cas9 nickase protein comprises the sequence of SEQ ID NO: 2;
the first linker comprises the sequence of SEQ ID NO: 31;
the MS2 binding protein comprises the sequence of SEQ ID NO: 21;
the second linker comprises the sequence of SEQ ID NO: 32;
the C-terminus portion of the Cas9 nickase protein comprises the sequence of SEQ ID NO: 11;
the third linker comprises the sequence of SEQ ID NO: 26; and
the NT protein comprises the sequence of SEQ ID NO: 19; or
I:
the N-terminus portion of the Cas9 nickase protein comprises the sequence of SEQ ID NO: 3;
the first linker comprises the sequence of SEQ ID NO: 34;
the first MS2 binding protein comprises the sequence of SEQ ID NO: 21;
the second linker comprises the sequence of SEQ ID NO: 31;
the second MS2 binding protein comprises the sequence of SEQ ID NO: 21;
the third linker comprises the sequence of SEQ ID NO: 33;
the C-terminus portion of the Cas9 nickase comprises the sequence of SEQ ID NO: 12;
the fourth linker comprises the sequence of SEQ ID NO: 26; and
the NT protein comprises the sequence of SEQ ID NO: 19; or
J:
the N-terminus portion of the Cas9 nickase protein comprises the sequence of SEQ ID NO: 4;
the first linker comprises the sequence of SEQ ID NO: 34;
the first MS2 binding protein comprises the sequence of SEQ ID NO: 21;
the second linker comprises the sequence of SEQ ID NO: 31;
the second MS2 binding protein comprises the sequence of SEQ ID NO: 21;
the third linker comprises the sequence of SEQ ID NO: 33;
the C-terminus portion of the Cas9 nickase comprises the sequence of SEQ ID NO: 13;
the fourth linker comprises the sequence of SEQ ID NO: 26; and
the NT protein comprises the sequence of SEQ ID NO: 19; or
K:
the N-terminus portion of the Cas9 nickase protein comprises the sequence of SEQ ID NO: 5;
the first linker comprises the sequence of SEQ ID NO: 34;
the first MS2 binding protein comprises the sequence of SEQ ID NO: 21;
the second linker comprises the sequence of SEQ ID NO: 31;
the second MS2 binding protein comprises the sequence of SEQ ID NO: 21;
the third linker comprises the sequence of SEQ ID NO: 33;
the C-terminus portion of the Cas9 nickase comprises the sequence of SEQ ID NO: 14;
the fourth linker comprises the sequence of SEQ ID NO: 26; and
the NT protein comprises the sequence of SEQ ID NO: 19; or
L:
the N-terminus portion of the Cas9 nickase protein comprises the sequence of SEQ ID NO: 6;
the first linker comprises the sequence of SEQ ID NO: 34;
the first MS2 binding protein comprises the sequence of SEQ ID NO: 21;
the second linker comprises the sequence of SEQ ID NO: 31;
the second MS2 binding protein comprises the sequence of SEQ ID NO: 21;
the third linker comprises the sequence of SEQ ID NO: 33;
the C-terminus portion of the Cas9 nickase comprises the sequence of SEQ ID NO: 15;
the fourth linker comprises the sequence of SEQ ID NO: 26; and
the NT protein comprises the sequence of SEQ ID NO: 19; or
M:
the N-terminus portion of the Cas9 nickase protein comprises the sequence of SEQ ID NO: 7;
the first linker comprises the sequence of SEQ ID NO: 34;
the first MS2 binding protein comprises the sequence of SEQ ID NO: 21;
the second linker comprises the sequence of SEQ ID NO: 31;
the second MS2 binding protein comprises the sequence of SEQ ID NO: 21;
the third linker comprises the sequence of SEQ ID NO: 33;
the C-terminus portion of the Cas9 nickase comprises the sequence of SEQ ID NO: 16;
the fourth linker comprises the sequence of SEQ ID NO: 26; and
the NT protein comprises the sequence of SEQ ID NO: 19; or
N:
the N-terminus portion of the Cas9 nickase protein comprises the sequence of SEQ ID NO: 8;
the first linker comprises the sequence of SEQ ID NO: 34;
the first MS2 binding protein comprises the sequence of SEQ ID NO: 21;
the second linker comprises the sequence of SEQ ID NO: 31
the second MS2 binding protein comprises the sequence of SEQ ID NO: 21;
the third linker comprises the sequence of SEQ ID NO: 33;
the C-terminus portion of the Cas9 nickase comprises the sequence of SEQ ID NO: 17;
the fourth linker comprises the sequence of SEQ ID NO: 26; and
the NT protein comprises the sequence of SEQ ID NO: 19.

29-41. (canceled)

42. The modular prime editing system of claim 1, wherein the fusion protein comprises the sequences of SEQ ID NOs: 43, 44, 45, 46, 47, 48, and 49.

43. A modular prime editing system, comprising:
i) a fusion protein comprising a Cas9 nickase protein linked to a nucleotide polymerase (NT) protein;
ii) a prime editor template RNA (petRNA) comprising a primer binding site, a nucleotide polymerase template (NPT), and at least one MS2 hairpin; and
iii) a single guide RNA (sgRNA)
wherein the fusion protein comprises at least four MS2 binding proteins.

44. The modular prime editing system of claim 43, wherein the fusion protein consists of four MS2 binding proteins, optionally wherein:
the fusion protein consists of four adjacent MS2 binding proteins on the N-terminus or C-terminus; or
the fusion protein consists of two MS2 binding proteins on the N-terminus, and two MS2 binding proteins on the C-terminus; or
the fusion protein consists of two MS2 binding proteins on the N-terminus or C-terminus, and two MS2 binding proteins inlaid in the Cas9 nickase.

45-88. (canceled)

89. The modular prime editing system of claim 43, wherein the fusion protein comprises from the N-terminus to the C-terminus:
four adjacent MS2 binding proteins, the Cas9 nickase protein, and an NT protein; or
a first MS2 binding protein, a second MS2 binding protein, the Cas9 nickase protein, an NT protein, a third MS2 binding protein and a fourth MS2 binding protein; or
a first MS2 binding protein, a second MS2 binding protein, the N-terminus portion of the Cas9 nickase protein, a third MS2 binding protein and a fourth MS2 binding protein, the C-terminus portion of the Cas9 nickase protein, and an NT protein; or
the Cas9 nickase protein, an NT protein, and four adjacent MS2 binding proteins; or
a first MS2 binding protein, a first linker, a second MS2 binding protein, a second linker, a third MS2 protein, a third linker, a fourth MS2 protein, a fourth linker, the Cas9 nickase protein, a fifth linker, and an NT protein; or
a first MS2 binding protein, a first linker, a second MS2 binding protein, a second linker, the Cas9 nickase protein, a third linker, an NT protein, a fourth linker, a third MS2 binding protein, a fifth linker, and a fourth MS2 protein; or
a first MS2 binding protein, a first linker, a second MS2 binding protein, a second linker, the N-terminus portion of the Cas9 nickase protein, a third linker, a third MS2 binding protein, a fourth linker, a fourth MS2 protein, a fifth linker, the C-terminus portion of the Cas9 nickase protein, and an NT protein, or
the Cas9 nickase protein, a first linker, and an NT protein, a second linker, a first MS2 binding protein, a third linker, a second MS2 binding protein, a fourth linker, a third MS2 protein, a fifth linker, and a fourth MS2 protein.

90-92. (canceled)

93. The modular prime editing system of claim 43, wherein:
the first MS2 binding protein comprises the sequence of SEQ ID NO: 21;
the second MS2 binding protein comprises the sequence of SEQ ID NO: 21;
the third MS2 binding protein comprises the sequence of SEQ ID NO: 21;
the fourth MS2 binding protein comprises the sequence of SEQ ID NO: 21;
the Cas9 nickase protein comprise the sequence of SEQ ID NO: 1; and
the NT comprises the sequence of SEQ ID NO: 19; or
the first MS2 binding protein comprises the sequence of SEQ ID NO: 21;
the second MS2 binding protein comprises the sequence of SEQ ID NO: 21
the Cas9 nickase protein comprise the sequence of SEQ ID NO: 1;
the NT comprises the sequence of SEQ ID NO: 19;
the third MS2 binding protein comprises the sequence of SEQ ID NO: 21; and
the fourth MS2 binding protein comprises the sequence of SEQ ID NO: 21; or
the first MS2 binding protein comprises the sequence of SEQ ID NO: 21;
the second MS2 binding protein comprises the sequence of SEQ ID NO: 21;
the N-terminus portion of the Cas9 nickase protein comprises the sequence of SEQ ID NO: 9;
the third MS2 binding protein comprises the sequence of SEQ ID NO: 21;
the fourth MS2 binding protein comprises the sequence of SEQ ID NO: 21;
the C-terminus portion of the Cas9 nickase protein comprises the sequence of SEQ ID NO: 18; and
the NT comprises the sequence of SEQ ID NO: 19; or
the first MS2 binding protein comprises the sequence of SEQ ID NO: 21;
the first linker comprises the sequence of SEQ ID NO: 31;
the second MS2 binding protein comprises the sequence of SEQ ID NO: 21;
the second linker comprises the sequence of SEQ ID NO: 33;
the third MS2 binding protein comprises the sequence of SEQ ID NO: 21;
the third linker comprises the sequence of SEQ ID NO: 31;
the fourth MS2 binding protein comprises the sequence of SEQ ID NO: 21;
the fourth linker comprises the sequence of SEQ ID NO: 30;
the Cas9 nickase protein comprise the sequence of SEQ ID NO: 1;
the fifth linker comprises the sequence of SEQ ID NO: 26, and
the NT comprises the sequence of SEQ ID NO: 19; or
the first MS2 binding protein comprises the sequence of SEQ ID NO: 21;
the first linker comprises the sequence of SEQ ID NO: 31;
the second MS2 binding protein comprises the sequence of SEQ ID NO: 21;
the second linker comprises the sequence of SEQ ID NO: 30;
the Cas9 nickase protein comprise the sequence of SEQ ID NO: 1;
the third linker comprises the sequence of SEQ ID NO: 26;
the NT comprises the sequence of SEQ ID NO: 19;
the fourth linker comprises the sequence of SEQ ID NO: 34;
the third MS2 binding protein comprises the sequence of SEQ ID NO: 21;
the fifth linker comprises the sequence of SEQ ID NO: 31; and
the fourth MS2 binding protein comprises the sequence of SEQ ID NO: 21; or
the first MS2 binding protein comprises the sequence of SEQ ID NO: 21;
the first linker comprises the sequence of SEQ ID NO: 31;
the second MS2 binding protein comprises the sequence of SEQ ID NO: 21;
the second linker comprises the sequence of SEQ ID NO: 30;
the N-terminus portion of the Cas9 nickase protein comprises the sequence of SEQ ID NO: 9;
the third linker comprises the sequence of SEQ ID NO: 34;
the third MS2 binding protein comprises the sequence of SEQ ID NO: 21;
the fourth linker comprises the sequence of SEQ ID NO: 31;
the fourth MS2 binding protein comprises the sequence of SEQ ID NO: 21;
the fifth linker comprises the sequence of SEQ ID NO: 30;
the C-terminus portion of the Cas9 nickase protein comprises the sequence of SEQ ID NO: 18;
the sixth linker comprises the sequence of SEQ ID NO: 26; and
the NT comprises the sequence of SEQ ID NO: 19.

94-98. (canceled)

99. The modular prime editing system of claim 43, wherein the fusion protein comprises the sequences of SEQ ID NOs: 50, 51, and 52.

100. A modular prime editing system, comprising:
i) a fusion protein comprising a Cas9 nickase protein linked to a nucleotide polymerase (NT) protein;
ii) a prime editor template RNA (petRNA) comprising a primer binding site, a nucleotide polymerase template (NPT), and at least one MS2 hairpin; and
iii) a single guide RNA (sgRNA)
wherein the fusion protein comprises at least one MS2 binding protein at the N terminus and at least one MS2 binding protein at the C terminus.

101-104. (canceled)

105. The modular prime editing system of claim 100, wherein the fusion protein comprises from the N-terminus to the C-terminus:
a first MS2 binding protein, the Cas9 nickase protein, an NT protein, and a second MS2 binding protein; and/or
a first MS2 binding protein, a first linker, the Cas9 nickase protein, a second linker, an NT protein, a third linker, and a second MS2 binding protein.

106-108. (canceled)

109. The modular prime editing system of claim 100, wherein:
the first MS2 binding protein comprises the sequence of SEQ ID NO: 21;
the Cas9 nickase protein comprises the sequence of SEQ ID NO: 1;
the NT comprises the sequence of SEQ ID NO: 19; and
the second MS2 binding protein comprises the sequence of SEQ ID NO: 21; and/or
the first MS2 binding protein comprises the sequence of SEQ ID NO: 21
the first linker comprises the sequence of SEQ ID NO: 30;
the Cas9 nickase protein comprises the sequence of SEQ ID NO: 1;
the second linker comprises the sequence of SEQ ID NO: 26;
the NT comprises the sequence of SEQ ID NO: 19;
the third linker comprises the sequence of SEQ ID NO: 26; and
the second MS2 binding protein comprises the sequence of SEQ ID NO: 21.

110. (canceled)

111. The modular prime editing system of claim 100, wherein the fusion protein comprises the sequence of SEQ ID NO: 42.

112. A modular prime editing system, comprising:
i) a fusion protein comprising a Cas9 nickase protein linked to a nucleotide polymerase (NT) protein;
ii) a prime editor template RNA (petRNA) comprising a primer binding site, a nucleotide polymerase template (NPT), and at least one MS2 hairpin; and
iii) a single guide RNA (sgRNA)
wherein the fusion protein comprises at least one MS2 binding protein at the N terminus or at least one MS2 binding protein at the C terminus or at least one MS2 binding protein between the Cas9 nickase and the RT.

113-122. (canceled)

123. The modular prime editing system of claim 112, wherein the fusion protein comprises from the N-terminus to the C-terminus:
an MS2 binding protein, the Cas9 nickase protein, and an NT protein; or
the Cas9 nickase protein, the NT protein, and an MS2 binding protein; or
the Cas9 nickase protein, an MS2 binding protein, and the NT protein; or
the MS2 binding protein, a first linker, the Cas9 nickase protein, a second linker and an NT protein; or
the Cas9 nickase protein, a first linker, the NT protein, a second linker, and an MS2 binding protein; or
the Cas9 nickase protein, a first linker, an MS2 binding protein, a second linker, and the NT protein.

124-134. (canceled)

135. The modular prime editing system of claim 112, wherein the fusion protein comprises the sequence of SEQ ID NO: 38, 39, 40, or 41.

136-138. (canceled)

139. The modular prime editing system of claim 1, wherein the petRNA is chemically modified.

140-164. (canceled)

165. A fusion protein comprising a Cas9 nickase protein linked to a nucleotide polymerase (NT) protein, wherein the fusion protein comprises at least one MS2 binding protein inlaid within said Cas9 nickase.

166-199. (canceled)

200. A polynucleotide sequence encoding the fusion protein of claim 165.

201-204. (canceled)

205. A host cell comprising the polynucleotide sequence of claim 200.

206. A method of delivering the modular prime editing system of claim 1 to a cell, the method comprising incubating the modular prime editing with the cell.

207-208. (canceled)

209. A method of editing a target gene in a cell, comprising administering to said cell the modular prime editing system of claim 1, optionally wherein:
the target gene is selected from the list comprising of: EXM1, HEXA, IDUA, HBB, VEGFA, RUNX1, PSEN1, IDS, FANCF, PRNP, and DNMT1; and/or
the sgRNA comprises from N-terminus to C-terminus a variable spacer sequence and a common scaffold sequence, optionally wherein the variable spacer sequence is selected from the sequences of SEQ ID(s) NO(s): 54-86.

210-214. (canceled)

215. A petRNA a comprising a primer binding site, a nucleotide polymerase template (NPT), at least one MS2 hairpin, and at least one chemically modified nucleotide.

216-225. (canceled)

226. The petRNA of claim 215, wherein said petRNA comprises one MS2 hairpin, optionally wherein the at least one MS2 hairpin is chemically modified.

227-240. (canceled)

241. The petRNA of claim 215, wherein the MS2 is linked to the RTT using a linker, optionally wherein the linker is selected from the group consisting of ethylene glycol and polyethylene glycol (PEG), optionally wherein the PEG is a hexaethylene glycol (HEX).

242-252. (canceled)

253. A petRNA a comprising a primer binding site, a nucleotide polymerase template (NPT), and at least one MS2 hairpin, wherein the MS2 is linked to the RTT using a linker.

254-264. (canceled)


SEQUENCE LISTING (114 sequences)

SEQ ID NO: 1 - protein, 1363 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 354 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQS

SEQ ID NO: 3 - protein, 1021 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSE

SEQ ID NO: 4 - protein, 1049 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLAN

SEQ ID NO: 5 - protein, 1242 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKG

SEQ ID NO: 6 - protein, 1294 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRD

SEQ ID NO: 7 - protein, 822 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQE

SEQ ID NO: 8 - protein, 789 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELG

SEQ ID NO: 9 - protein, 1242 aa, synthetic construct; identical to SEQ ID NO: 5

SEQ ID NO: 10 - skipped

SEQ ID NO: 11 - protein, 1009 aa, synthetic construct
KNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 342 aa, synthetic construct
QEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 13 - protein, 314 aa, synthetic construct
GEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 121 aa, synthetic construct
SPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 15 - protein, 69 aa, synthetic construct
KPIREQAENIIHLFTLTNLGAPAAFKYFDTTIDRKRYTSTKEVLDATLIHQSITGLYETRIDLSQLGGD

SEQ ID NO: 16 - protein, 541 aa, synthetic construct
LDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - protein, 463 aa, synthetic construct
GGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 121 aa, synthetic construct; identical to SEQ ID NO: 14

SEQ ID NO: 19 - protein, 677 aa, Murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 20 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 21 - protein, 129 aa, synthetic construct
ASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 22 - protein, 19 aa, synthetic construct
MKRTADGSEFESPKKKRKV

SEQ ID NO: 23 - protein, 17 aa, synthetic construct
KRTADGSEFEPKKKRKV

SEQ ID NO: 24 - protein, 7 aa, synthetic construct
PKKKRKV

SEQ ID NO: 25 - protein, 16 aa, synthetic construct
KRPAATKKAGQAKKKK

SEQ ID NO: 26 - protein, 33 aa, synthetic construct
SGGSSGGSSGSETPGTSESATPESSGGSSGGSS

SEQ ID NO: 27 - protein, 4 aa, synthetic construct
SGGS

SEQ ID NO: 28 - skipped

SEQ ID NO: 29 - protein, 8 aa, synthetic construct
GIHGVPAA

SEQ ID NO: 30 - protein, 30 aa, synthetic construct
SAGGGGSGGGGSGGGGSGPKKKRKVAAAGS

SEQ ID NO: 31 - protein, 20 aa, synthetic construct
GSSGSETPGTSESATPESSG

SEQ ID NO: 32 - protein, 20 aa, synthetic construct
GGSGGSGGSGGSGGSGGSGG

SEQ ID NO: 33 - protein, 18 aa, synthetic construct
SAGGGGSGGGGSGGGGSG

SEQ ID NO: 34 - protein, 16 aa, synthetic construct
SGGSSGGSSGGSSGGS

SEQ ID NO: 35 - protein, 2113 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 36 - protein, 1419 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 37 - protein, 861 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 38 - protein, 2275 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 39 - protein, 2275 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSKRTADGSEFEPKKKRKV

SEQ ID NO: 40 - protein, 2272 aa, synthetic construct
MKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 41 - protein, 2421 aa, synthetic construct
MKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 42 - protein, 2434 aa, synthetic construct
MKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSKRTADGSEFEPKKKRKV

SEQ ID NO: 43 - protein, 2282 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGSGGSGGSGGSGGSGGSGGKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 44 - protein, 2425 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSESGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 45 - protein, 2425 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANSGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 46 - protein, 2425 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 47 - protein, 2425 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDSGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 48 - protein, 2425 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQESGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGLDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 49 - protein, 2314 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 50 - protein, 2717 aa, synthetic construct
MKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 51 - protein, 2715 aa, synthetic construct
MKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSKRTADGSEFEPKKKRKV

SEQ ID NO: 52 - protein, 2733 aa, synthetic construct
MKRTADGSEFESPKKKRKVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSGGSSGGSSGGSSGGSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSSGSETPGTSESATPESSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 53 - protein, 23 aa, synthetic construct
MDYKDHDGDYKDHDIDYKDDDDK

SEQ ID NO: 54 - RNA, 20 nt, synthetic construct
  source 1..20: mol_type = other RNA
  ncRNA 1..20: ncRNA_class = sgRNA
aagttcagcgtgtccggctt

SEQ ID NO: 55 - RNA, 20 nt, synthetic construct
  source 1..20: mol_type = other RNA
  ncRNA 1..20: ncRNA_class = sgRNA
gagtccgagcagaagaagaa

SEQ ID NO: 56 - RNA, 20 nt, synthetic construct
  source 1..20: mol_type = other RNA
  ncRNA 1..20: ncRNA_class = sgRNA
tacctgaaccgtatatccta

SEQ ID NO: 57 - RNA, 20 nt, synthetic construct
  source 1..20: mol_type = other RNA
  ncRNA 1..20: ncRNA_class = sgRNA
ccgcagatgaggagcagctc

SEQ ID NO: 58 - RNA, 20 nt, synthetic construct
  source 1..20: mol_type = other RNA
  ncRNA 1..20: ncRNA_class = sgRNA
catggtgcacctgactcctg

SEQ ID NO: 59 - RNA, 20 nt, synthetic construct
  source 1..20: mol_type = other RNA
  ncRNA 1..20: ncRNA_class = sgRNA
gatgtctgcaggccagatga

SEQ ID NO: 60 - RNA, 20 nt, synthetic construct
  source 1..20: mol_type = other RNA
  ncRNA 1..20: ncRNA_class = sgRNA
gcattttcaggaggaagcga

SEQ ID NO: 61 - RNA, 20 nt, synthetic construct
  source 1..20: mol_type = other RNA
  ncRNA 1..20: ncRNA_class = sgRNA
aaagagcatgatcacatgct

SEQ ID NO: 62 - RNA, 20 nt, synthetic construct
  source 1..20: mol_type = other RNA
  ncRNA 1..20: ncRNA_class = sgRNA
actgagggatgtctgaaggc

SEQ ID NO: 63 - RNA, 20 nt, synthetic construct
  source 1..20: mol_type = other RNA
  ncRNA 1..20: ncRNA_class = sgRNA
ggaatcccttctgcagcacc

SEQ ID NO: 64 - RNA, 20 nt, synthetic construct
  source 1..20: mol_type = other RNA
  ncRNA 1..20: ncRNA_class = sgRNA
gcagtggtggggggccttgg

SEQ ID NO: 65 - RNA, 20 nt, synthetic construct
  source 1..20: mol_type = other RNA
  ncRNA 1..20: ncRNA_class = sgRNA
gattcctggtgccagaaaca

SEQ ID NO: 66 - RNA, 20 nt, synthetic construct
  source 1..20: mol_type = other RNA
  ncRNA 1..20: ncRNA_class = sgRNA
gcggtagctcccagaacggt

SEQ ID NO: 67 - RNA, 20 nt, synthetic construct
  source 1..20: mol_type = other RNA
  ncRNA 1..20: ncRNA_class = sgRNA
tgacttccatggttccacaa

SEQ ID NO: 68 - RNA, 20 nt, synthetic construct
  source 1..20: mol_type = other RNA
  ncRNA 1..20: ncRNA_class = sgRNA
cgggctggagctgttcgcgc

SEQ ID NO: 69 - RNA, 20 nt, synthetic construct
  source 1..20: mol_type = other RNA
  ncRNA 1..20: ncRNA_class = sgRNA
tacctgaaccgtgtaaagta

SEQ ID NO: 70 - RNA, 20 nt, synthetic construct
  source 1..20: mol_type = other RNA
  ncRNA 1..20: ncRNA_class = sgRNA
cccataccttggagcaacgg

SEQ ID NO: 71 - RNA, 20 nt, synthetic construct
  source 1..20: mol_type = other RNA
  ncRNA 1..20: ncRNA_class = sgRNA
gacatcgatgtcctccccat

SEQ ID NO: 72 - RNA, 20 nt, synthetic construct
  source 1..20: mol_type = other RNA
  ncRNA 1..20: ncRNA_class = sgRNA
gctttcaccttcaaatgcca

SEQ ID NO: 73 - RNA, 20 nt, synthetic construct
  source 1..20: mol_type = other RNA
  ncRNA 1..20: ncRNA_class = sgRNA
ggccgggccctgggggcggt

SEQ ID NO: 74 - RNA, 20 nt, synthetic construct
  source 1..20: mol_type = other RNA
  ncRNA 1..20: ncRNA_class = sgRNA
ccttgataccaacctgccca

SEQ ID NO: 75 - RNA, 20 nt, synthetic construct
  source 1..20: mol_type = other RNA
  ncRNA 1..20: ncRNA_class = sgRNA
atgtacagagagcccagggc

SEQ ID NO: 76 - RNA, 20 nt, synthetic construct
  source 1..20: mol_type = other RNA
  ncRNA 1..20: ncRNA_class = sgRNA
tcacctctcatgaagcactg

SEQ ID NO: 77 - RNA, 20 nt, synthetic construct
  source 1..20: mol_type = other RNA
  ncRNA 1..20: ncRNA_class = sgRNA
ttatctaatggacgacccca

SEQ ID NO: 78 - RNA, 20 nt, synthetic construct
  source 1..20: mol_type = other RNA
  ncRNA 1..20: ncRNA_class = sgRNA
gcattttcgattccgtgact

SEQ ID NO: 79 - RNA, 20 nt, synthetic construct
  source 1..20: mol_type = other RNA
  ncRNA 1..20: ncRNA_class = sgRNA
ggggtcccaggtgctgacgt

SEQ ID NO: 80 - RNA, 20 nt, synthetic construct
  source 1..20: mol_type = other RNA
  ncRNA 1..20: ncRNA_class = sgRNA
gcatgttttcacgatagtaa

SEQ ID NO: 81 - RNA, 20 nt, synthetic construct
  source 1..20: mol_type = other RNA
  ncRNA 1..20: ncRNA_class = sgRNA
tttcccttcagctaaaataa

SEQ ID NO: 82 - RNA, 20 nt, synthetic construct
  source 1..20: mol_type = other RNA
  ncRNA 1..20: ncRNA_class = sgRNA
ggtgaccggaggcatatgga

SEQ ID NO: 83 - RNA, 20 nt, synthetic construct
  source 1..20: mol_type = other RNA
  ncRNA 1..20: ncRNA_class = sgRNA
ccagcccagcacataatgga

SEQ ID NO: 84 - RNA, 20 nt, synthetic construct
  source 1..20: mol_type = other RNA
  ncRNA 1..20: ncRNA_class = sgRNA
gtcgtctgcaacctgcaaga

SEQ ID NO: 85 - RNA, 20 nt, synthetic construct
  source 1..20: mol_type = other RNA
  ncRNA 1..20: ncRNA_class = sgRNA
gctctcaccatgaaacgcca

SEQ ID NO: 86 - RNA, 20 nt, synthetic construct
  source 1..20: mol_type = other RNA
  ncRNA 1..20: ncRNA_class = sgRNA
gttggggtgatgctcttcgg

SEQ ID NO: 87 - RNA, 23 nt, synthetic construct
  source 1..23: mol_type = other RNA
  ncRNA 1..23: ncRNA_class = other; note = petRNA
gcacatgaggatcacccatgtgc

SEQ ID NO: 88 - RNA, 30 nt, synthetic construct
  source 1..30: mol_type = other RNA
  ncRNA 1..30: ncRNA_class = other; note = petRNA
tttgtctcgccaaaggtctccggacacgct

SEQ ID NO: 89 - RNA, 65 nt, synthetic construct
  source 1..65: mol_type = other RNA
  ncRNA 1..65: ncRNA_class = other; note = petRNA
gtgcagatgaacttcagggtcagcttgccgtaggtggcatcgccctcgccctcgccggacacgct

SEQ ID NO: 90 - RNA, 30 nt, synthetic construct
  source 1..30: mol_type = other RNA
  ncRNA 1..30: ncRNA_class = other; note = petRNA
gtgatgggagcacttcttcttctgctcgga

SEQ ID NO: 91 - RNA, 30 nt, synthetic construct
  source 1..30: mol_type = other RNA
  ncRNA 1..30: ncRNA_class = other; note = petRNA
agtcagggccataggatagatatacggttc

SEQ ID NO: 92 - RNA, 28 nt, synthetic construct
  source 1..28: mol_type = other RNA
  ncRNA 1..28: ncRNA_class = other; note = petRNA
acacttcggcctagagctgctcctcatc

SEQ ID NO: 93 - RNA, 27 nt, synthetic construct
  source 1..27: mol_type = other RNA
  ncRNA 1..27: ncRNA_class = other; note = petRNA
agacttctctacaggagtcaggtgcac

SEQ ID NO: 94 - RNA, 35 nt, synthetic construct
  source 1..35: mol_type = other RNA
  ncRNA 1..35: ncRNA_class = other; note = petRNA
aatgtgccatctggagcagtgatctggcctgcaga

SEQ ID NO: 95 - RNA, 30 nt, synthetic construct
  source 1..30: mol_type = other RNA
  ncRNA 1..30: ncRNA_class = other; note = petRNA
tgtctgaagcaatcgcttcctcctgaaaat

SEQ ID NO: 96 - RNA, 31 nt, synthetic construct
  source 1..31: mol_type = other RNA
  ncRNA 1..31: ncRNA_class = other; note = petRNA
aaatatggcgtcaagcatgtgatcatgctct

SEQ ID NO: 97 - RNA, 29 nt, synthetic construct
  source 1..29: mol_type = other RNA
  ncRNA 1..29: ncRNA_class = other; note = petRNA
gccagtatccctggccttcagacatccct

SEQ ID NO: 98 - RNA, 31 nt, synthetic construct
  source 1..31: mol_type = other RNA
  ncRNA 1..31: ncRNA_class = other; note = petRNA
ggaaaagcgatcgtgatgctgcagaagggat

SEQ ID NO: 99 - RNA, 24 nt, synthetic construct
  source 1..24: mol_type = other RNA
  ncRNA 1..24: ncRNA_class = other; note = petRNA
atgtagacgccaaggccccccacc

SEQ ID NO: 100 - RNA, 28 nt, synthetic construct
  source 1..28: mol_type = other RNA
  ncRNA 1..28: ncRNA_class = other; note = petRNA
tcccgtcaccactgtttctggcaccagg

SEQ ID NO: 101 - RNA, 23 nt, synthetic construct
  source 1..23: mol_type = other RNA
  ncRNA 1..23: ncRNA_class = other; note = petRNA
gatggccaccgttctgggagcta

SEQ ID NO: 102 - RNA, 26 nt, synthetic construct
  source 1..26: mol_type = other RNA
  ncRNA 1..26: ncRNA_class = other; note = petRNA
aatggacggattgtggaaccatggaa

SEQ ID NO: 103 - RNA, 25 nt, synthetic construct
  source 1..25: mol_type = other RNA
  ncRNA 1..25: ncRNA_class = other; note = petRNA
aagatggcagcgcgaacagctccag

SEQ ID NO: 104 - RNA, 30 nt, synthetic construct
  source 1..30: mol_type = other RNA
  ncRNA 1..30: ncRNA_class = other; note = petRNA
agtcagggccatacgatatttacacggttc

SEQ ID NO: 105 - RNA, 35 nt, synthetic construct
  source 1..35: mol_type = other RNA
  ncRNA 1..35: ncRNA_class = other; note = petRNA
accgccaccttccgccggtaattgctccaaggtat

SEQ ID NO: 106 - RNA, 76 nt, synthetic construct
  source 1..76: mol_type = other RNA
  ncRNA 1..76: ncRNA_class = sgRNA
gttttagagctagaaatagcaagttaaaataaggctagtccgttatcaacttgaaaaagtggcaccgagtcggtgc

SEQ ID NO: 107 - RNA, 23 nt, synthetic construct
  source 1..23: mol_type = other RNA
  modified_base 1..3: mod_base = OTHER; note = 2-Prime-O-methyl nucleotides
  modified_base 1..4: mod_base = OTHER; note = Phosphorothioate internucleotide linkage
  ncRNA 1..23: ncRNA_class = other; note = petRNA
gcacatgaggatcacccatgtgc

SEQ ID NO: 108 - RNA, 23 nt, synthetic construct
  source 1..23: mol_type = other RNA
  modified_base 1..10: mod_base = OTHER; note = 2-Prime-O-methyl nucleotides
  modified_base 1..4: mod_base = OTHER; note = Phosphorothioate internucleotide linkage
  ncRNA 1..23: ncRNA_class = other; note = petRNA
gcacatgaggatcacccatgtgc

SEQ ID NO: 109 - RNA, 23 nt, synthetic construct
  source 1..23: mol_type = other RNA
  modified_base 1..23: mod_base = OTHER; note = 2-Prime-O-methyl nucleotides
  modified_base 1..4: mod_base = OTHER; note = Phosphorothioate internucleotide linkage
  ncRNA 1..23: ncRNA_class = other; note = petRNA
gcacatgaggatcacccatgtgc

SEQ ID NO: 110 - RNA, 19 nt, synthetic construct
  source 1..19: mol_type = other RNA
  ncRNA 1..19: ncRNA_class = other; note = petRNA
tttgtctcgccaaaggtct

SEQ ID NO: 111 - RNA, 11 nt, synthetic construct
  source 1..11: mol_type = other RNA
  modified_base 1..11: mod_base = OTHER; note = 2-Prime-O-methyl nucleotides
  modified_base 8..11: mod_base = OTHER; note = Phosphorothioate internucleotide linkage
  ncRNA 1..11: ncRNA_class = other; note = petRNA
ccggacacgct

SEQ ID NO: 112 - protein, 6 aa, synthetic construct
HHHHHH

SEQ ID NO: 113 - DNA, 19 bp, synthetic construct
  source 1..19: mol_type = other DNA
taatacgactcactataag

SEQ ID NO: 114 - RNA, 110 nt, synthetic construct
  source 1..110: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa